(19) United States
(12) Patent Application Publication
(10) Pub. No.: US 2026/0364282 A1
(43) Pub. Date: Nov. 19, 2026

(54) TENSIONING MECHANISMS FOR PEELING A SHEET FROM A BLISTER STRIP FOR USE IN DRY POWDER INHALERS

(71) Applicant: TRANSPIRE BIO INC., Weston, FL (US)

(72) Inventors: Thomas Alexander EARWAKER, Leamington Spa (GB); Zoe Georgina ARMSTRONG, Leamington Spa (GB); Michael Ray NICHOLSON-LEWIS, Leamington Spa (GB); Matthew Meredith JONES, Warwick (GB); Jianye WANG, St. Albans (GB); James Alexander SENIOR, Warwick (GB)

(21) Appl. No.: 19/153,777

(22) PCT Filed: Feb. 1, 2024
(86) PCT No.: PCT/US2024/014004

§ 371 Date: Aug. 5, 2025

Related U.S. Application Data
(60) Provisional application No. 63/483,383, filed on Feb. 6, 2023.

(51) Int. Cl. A61M 15/00

(52) U.S. Cl. CPC A61M 15/0043; A61M 15/0051; A61M 2202/064

(57) ABSTRACT

Inhaler devices for delivering dry powdered medicament from at least one blister strip. The inhaler device includes an actuator for operating a dispensing mechanism of the inhaler device. Tensioning mechanisms for use in the inhaler devices are configured to maintain consistent tension in the blister strip over a lifetime of the device.

### CROSS REFERENCE OF RELATED APPLICATIONS

This application claims the benefit of U.S. Provisional Patent Application No. 63/483383, filed Feb. 6, 2023, which is hereby incorporated by reference in its entirety for all purposes.

### FIELD OF THE INVENTION

The invention relates in general to an inhaler device, and more particularly to tensioning mechanisms for use in an inhaler device configured to dispense dry power medicament from one or more blister strips.

### BACKGROUND OF THE INVENTION

Medicaments may be administered to a patient by inhalation using dry powder dispenser devices. Such devices are often used for the treatment and prophylaxis of respiratory diseases, including but not limited to asthma and chronic obstructive pulmonary disease (COPD). The medicament carrier may include a blister strip containing several discrete doses of powdered medicament. Such devices typically contain a mechanism such as piercing means for accessing a medicament dose by opening one or more blister pockets. The powdered medicament can then be accessed by the device and inhaled via the device by the user.

For inhalation or inhaler devices of the type described above to properly function, it is necessary to maintaining consistent tension in the blister strip over the lifetime of the device. Embodiments hereof relate to mechanisms for maintaining consistent tension in the blister strip in a dry powder inhaler device.

### BRIEF SUMMARY OF THE INVENTION

According to a first embodiment hereof, the present disclosure provides a tensioning mechanism for peeling a sheet from a blister strip for use in a dry powder inhaler device. The tensioning mechanism includes a base, a nut that includes at least one rib that engages with a cam surface, a compression spring longitudinally axially adjacent to the nut, and a take-up hub disposed about the nut and the compression spring. The nut is disposed between and coupled to each of the take-up hub and the base. The take-up hub is rotationally constrained to the nut and is configured to rotate relative to the base. When the base is rotationally driven, the nut is configured to interact with the compression spring and the cam surface to apply a torque onto the take-up hub. When the take-up hub applies an opposing torque onto the nut, the nut moves along the cam surface and axially compresses the compression spring.

In an aspect of the first embodiment, and in combination with any other aspects herein, the disclosure provides that the take-up hub includes a hook on an outer surface thereof. The hook is configured to attach to an end of the sheet of the blister strip such that rotation of the take-up hub results in winding of the sheet of the blister strip around the take-up hub.

In an aspect of the first embodiment, and in combination with any other aspects herein, the disclosure provides that the take-up hub is axially constrained relative to the base.

In an aspect of the first embodiment, and in combination with any other aspects herein, the disclosure provides that the take-up hub is axially constrained relative to the base via a bayonet connection between the base and the take-up hub.

In an aspect of the first embodiment, and in combination with any other aspects herein, the disclosure provides that the bayonet connection includes a radial extension on the base and an internal flange in the take-up hub, the internal flange including an axial slot that is configured to allow passage of the radial extension therethrough.

In an aspect of the first embodiment, and in combination with any other aspects herein, the disclosure provides that the base includes a plurality of gear teeth integrally formed with or fixed to an outer circumferential surface of the base.

In an aspect of the first embodiment, and in combination with any other aspects herein, the disclosure provides that the cam surface includes alternating sections of vertical surfaces and inclined surfaces.

In an aspect of the first embodiment, and in combination with any other aspects herein, the disclosure provides that the at least one rib includes a plurality of ribs circumferentially spaced apart.

In an aspect of the first embodiment, and in combination with any other aspects herein, the disclosure provides that the cam surface is integrally formed with or fixed to a portion of the base. The at least one rib projects radially inwards from an inner circumferential surface of the nut, and the take-up hub is rotationally locked to the nut.

In an aspect of the first embodiment, and in combination with any other aspects herein, the disclosure provides that the compression spring extends between the take-up hub and the nut.

In an aspect of the first embodiment, and in combination with any other aspects herein, the disclosure provides that the take-up hub is rotationally locked to the nut via an outwardly-extending rib which projects radially outwards from an outer circumferential surface of the nut and is received within an axial slot of the take-up hub.

In an aspect of the first embodiment, and in combination with any other aspects herein, the disclosure provides that the cam surface has a first outer diameter, and the compression spring has a second outer diameter, the first outer diameter being greater than the second outer diameter.

In an aspect of the first embodiment, and in combination with any other aspects herein, the disclosure provides that the cam surface is integrally formed with or fixed to a portion of the take-up hub. The at least one rib projects radially outwards from an outer circumferential surface of the nut, and the base is rotationally locked to the nut.

In an aspect of the first embodiment, and in combination with any other aspects herein, the disclosure provides that a shaft extends from the base, the shaft being integrally formed with or fixed to the base. The base is rotationally locked to the nut via an inwardly-extending rib which projects radially inwards from an inner circumferential surface of the nut and is received within an axial slot of the shaft.

In an aspect of the first embodiment, and in combination with any other aspects herein, the disclosure provides that the compression spring extends between the nut and the base.

According to a second embodiment hereof, the present disclosure provides a dry powder inhaler that includes a housing, an index spool, a take-up hub, and a tensioning mechanism. The housing receives at least one blister strip for use in the dry powder inhaler device, the blister strip including a bottom sheet and a top sheet releasably secured to the bottom sheet. The index spool is rotationally driven in a first direction, and an outer surface of the index spool receives the bottom sheet of the blister strip. The take-up hub is rotationally driven in the first direction or a second opposing direction, and an outer surface of the take-up hub is attached to an end of the top sheet and rotation of the take-up hub causes the top sheet to wind around the outer surface of the take-up hub. The tensioning mechanism includes a slider and at least one spring attached to the slider. The tensioning mechanism is coupled to the housing so as to permit axial movement of the slider relative to the housing along a predetermined path. The slider is configured to receive an intermediate portion of the top sheet of the blister strip, the intermediate portion of the top sheet disposed between the index spool and the take-up hub. Increased tension along the top sheet of the blister strip results in axial movement of the slider along the predetermined path, and axial movement of the slider axially compresses or extends the spring to reduce the tension along the top sheet of the blister strip.

In an aspect of the second embodiment, and in combination with any other aspects herein, the disclosure provides that a first end of the spring is attached to the slider and a second end of the spring is attached to the housing.

In an aspect of the second embodiment, and in combination with any other aspects herein, the disclosure provides that the predetermined path is formed by a recess in an inner surface of the housing.

In an aspect of the second embodiment, and in combination with any other aspects herein, the disclosure provides that the spring is a compression spring, and the compression spring is biased to push the slider away from each of the index spool and the take-up hub.

In an aspect of the second embodiment, and in combination with any other aspects herein, the disclosure provides that axial compression of the compression spring moves the slider closer to the each of the index spool and the take-up hub.

In an aspect of the second embodiment, and in combination with any other aspects herein, the disclosure provides that the predetermined path is linear.

In an aspect of the second embodiment, and in combination with any other aspects herein, the disclosure provides that the at least one spring includes a single spring.

In an aspect of the second embodiment, and in combination with any other aspects herein, the disclosure provides that the at least one spring includes two springs.

In an aspect of the second embodiment, and in combination with any other aspects herein, the disclosure provides that the take-up hub is rotationally driven in the first direction.

In an aspect of the second embodiment, and in combination with any other aspects herein, the disclosure provides that the take-up hub is rotationally driven in the second opposing direction.

In an aspect of the second embodiment, and in combination with any other aspects herein, the disclosure provides that the at least one spring is a compression spring and axial movement of the slider axially compresses the spring to reduce the tension along the top sheet of the blister strip.

In an aspect of the second embodiment, and in combination with any other aspects herein, the disclosure provides that the at least one spring is an extension spring and axial movement of the slider axially extends the spring to reduce the tension along the top sheet of the blister strip.

According to a third embodiment hereof, the present disclosure provides a dry powder inhaler that includes a housing, an index spool, a take-up hub, and a tensioning mechanism. The housing receives at least one blister strip for use in the dry powder inhaler device, the blister strip including a bottom sheet and a top sheet releasably secured to the bottom sheet. The housing includes a curved slot on an inner surface thereof and a pin radially extending from the inner surface of the housing, the pin disposed adjacent to a first end of the curved slot and fixed to the housing. An index spool is rotationally driven in a first direction, and an outer surface of the index spool receives the bottom sheet of the blister strip. The take-up hub which is rotationally driven in a second opposing direction. An outer surface of the take-up hub is attached to an end of the top sheet and rotation of the take-up hub causes the top sheet to wind around the outer surface of the take-up hub. The take-up hub is coupled to the housing so as to permit movement of the take-up hub relative to the housing along the curved slot. The tensioning mechanism includes at least one spring coupled to the take-up hub. A first end of the spring is fixed to the housing and a second end of the spring is coupled to the take-up hub. The pin is configured to receive an intermediate portion of the top sheet of the blister strip, the intermediate portion of the top sheet extending between the index spool and the take-up hub. Increased tension along the top sheet of the blister strip results in movement of the take-up hub along the curved slot of the housing, and movement of the take-up hub axially deforms the spring to reduce the tension along the top sheet of the blister strip.

In an aspect of the third embodiment, and in combination with any other aspects herein, the disclosure provides that the at least one spring is an extension spring.

In an aspect of the third embodiment, and in combination with any other aspects herein, the disclosure provides that the tensioning mechanism includes a bracket, the bracket having a first end coupled to permit relative rotation of the index spool relative to the bracket, a second end attached to the second end of the spring, and an intermediate portion coupled to the take-up hub to permit relative rotation of the take-up hub relative to the bracket.

In an aspect of the third embodiment, and in combination with any other aspects herein, the disclosure provides that the bracket is permitted to rotate relative to the housing.

In an aspect of the third embodiment, and in combination with any other aspects herein, the disclosure provides that the extension spring is biased to position the take-up hub at a second end of the curved slot which is opposite from the first end of the curved slot and the pin. Movement of the take-up hub in a direction towards the pin axially extends the extension spring to reduce the tension along the top sheet of the blister strip.

In an aspect of the third embodiment, and in combination with any other aspects herein, the disclosure provides that the extension spring is disposed in line with a centerline of the top sheet.

In an aspect of the third embodiment, and in combination with any other aspects herein, the disclosure provides that the curved slot is concentric with an axis of rotation of the index spool.

In an aspect of the third embodiment, and in combination with any other aspects herein, the disclosure provides that the at least one spring is a torsion spring.

In an aspect of the third embodiment, and in combination with any other aspects herein, the disclosure provides that the at least one spring includes a first torsion spring and a second torsion spring, the first torsion spring configured to act on a first side of the take-up hub and the second torsion spring configured to act on a second opposing side of the take-up hub.

In an aspect of the third embodiment, and in combination with any other aspects herein, the disclosure provides that a first leg of the torsion spring is fixed to the inner surface of the housing and a second leg of the torsion spring is coupled to the take-up hub to move in conjunction with the take-up hub along the curved slot.

In an aspect of the third embodiment, and in combination with any other aspects herein, the disclosure provides that a body of the torsion spring is disposed concentric to an axis of rotation of the index spool.

In an aspect of the third embodiment, and in combination with any other aspects herein, the disclosure provides that the torsion spring is biased to position the take-up hub at a second end of the curved slot which is opposite from the first end of the curved slot and the pin and movement of the take-up hub in a direction towards the pin twists the torsion spring to reduce the tension along the top sheet of the blister strip.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1A is a front view of an inhaler device according to an embodiment hereof, wherein a mouthpiece cover of the inhaler device is in a closed position.

FIG. 1B is a rear view of the inhaler device of FIG. 1A, wherein the mouthpiece cover of the inhaler device is in the closed position.

FIG. 1C is a front view of the inhaler device of FIG. 1A, wherein the mouthpiece cover of the inhaler device is in an open position.

FIG. 1D is a graph that illustrates an estimated actuation force profile for the inhaler device and mouthpiece cover as depicted in FIGS. 1A-1C (in the dotted line), compared to an estimated actuation force profile for an inhaler device in which a dispensing mechanism is not actuated for an initial period of travel of a mouthpiece cover (in the solid line).

FIG. 2 is a perspective view of two blister strips for use within the inhaler device of FIG. 1A.

FIG. 3A is a front view of the inhaler device of FIG. 1A, wherein the mouthpiece cover of the inhaler device is in an open position and a housing of the inhaler device is removed for sake of illustration only.

FIG. 3B is a sectional perspective view of a portion of the inhaler device of FIG. 1A illustrating a portion of the airflow path through the inhaler device of FIG. 1A.

FIG. 4 is a perspective view of a manifold of the inhaler device of FIG. 1A, wherein the manifold is removed from the inhaler device for sake of illustration only.

FIG. 4A is a sectional view taken along line A-A of FIG. 4.

FIG. 5 is a perspective view of the manifold of FIG. 4 positioned adjacent to inlet vents of the housing of the inhaler device of FIG. 1A.

FIG. 6 is another perspective view of the manifold of the inhaler device of FIG. 1A, wherein the manifold is removed from the inhaler device for sake of illustration only.

FIG. 7 is a schematic view illustrating an airflow path through the manifold of the inhaler device of FIG. 1A.

FIG. 8 is a schematic flowchart illustrating the airflow path through the manifold of the inhaler device of FIG. 1A.

FIG. 9A is a front view of the inhaler device of FIG. 1A, wherein the mouthpiece cover of the inhaler device is in the open position and a portion of the housing of the inhaler device is removed for sake of illustration only.

FIG. 9B is a rear view of the inhaler device of FIG. 1A, wherein the mouthpiece cover and the housing of the inhaler device are removed for sake of illustration only.

FIG. 10 is a front view of a ratchet mechanism of the inhaler device of FIG. 1A, wherein the ratchet gear is removed from the inhaler device for sake of illustration only.

FIG. 10A illustrates the ratchet mechanism of FIG. 10 when the mouthpiece cover is in the closed position.

FIG. 10B illustrates the ratchet mechanism of FIG. 10 when the mouthpiece cover is in the open position.

FIG. 11 is a perspective view of the mouthpiece cover and a portion of a dispensing subassembly of the inhaler device of FIG. 1A, wherein the mouthpiece cover and dispensing subassembly are removed from the inhaler device for sake of illustration only.

FIG. 12A is a perspective view of a counter subassembly of the inhaler device of FIG. 1A, wherein the counter subassembly is removed from the inhaler device for sake of illustration only.

FIG. 12B is a front view of the counter subassembly of FIG. 12A.

FIG. 12C is a sectional view of the counter subassembly of FIG. 12A, taken along line C-C of FIG. 12B.

FIG. 12D is a sectional view of the counter subassembly of FIG. 12A, taken along line D-D of FIG. 12C.

FIG. 13A is a schematic illustration of a tensioning mechanism of the inhaler device of FIG. 1A, wherein the tensioning mechanism is shown at a beginning of the device life.

FIG. 13B is a schematic illustration of a tensioning mechanism of the inhaler device of FIG. 1A, wherein the tensioning mechanism is shown near an end of the device life.

FIG. 14A is a perspective view of a tensioning mechanism of the inhaler device of FIG. 1A, wherein the tensioning mechanism is removed from the inhaler device for sake of illustration only.

FIG. 14B is a sectional view of the tensioning mechanism of FIG. 14A.

FIG. 14C is a cross-sectional view of the tensioning mechanism of FIG. 14A.

FIG. 14D is a perspective exploded view of a base and a nut of the tensioning mechanism of FIG. 14A.

FIG. 14E is a series of sectional views of the tensioning mechanism of FIG. 14A illustrating the movement of the nut during operation of the inhaler device.

FIG. 15A is a sectional view of a tensioning mechanism according to another embodiment hereof for use in the inhaler device of FIG. 1A, wherein the tensioning mechanism is removed from the inhaler device for sake of illustration only.

FIG. 15B is a perspective view of the tensioning mechanism of FIG. 15A, wherein a hub of the tensioning mechanism is removed for sake of illustration.

FIG. 16A is a sectional view of a tensioning mechanism according to another embodiment hereof for use in the inhaler device of FIG. 1A, wherein the tensioning mechanism is removed from the inhaler device for sake of illustration only.

FIG. 16B is a perspective view of the tensioning mechanism of FIG. 16A, wherein a hub of the tensioning mechanism is removed for sake of illustration.

FIG. 17A is a sectional view of a tensioning mechanism according to another embodiment hereof for use in the inhaler device of FIG. 1A, wherein the tensioning mechanism is removed from the inhaler device for sake of illustration only.

FIG. 17B is a perspective view of the tensioning mechanism of FIG. 17A, wherein a hub of the tensioning mechanism is removed for sake of illustration.

FIG. 18 is a side view of a tensioning mechanism according to another embodiment hereof for use in the inhaler device of FIG. 1A, wherein the tensioning mechanism is removed from the inhaler device for sake of illustration only.

FIG. 19A is a side view of the tensioning mechanism of FIG. 18, wherein the tensioning mechanism is in a first position.

FIG. 19B is a side view of the tensioning mechanism of FIG. 18, wherein the tensioning mechanism is in a second position.

FIG. 20 is a graph of performance characteristics of the tensioning mechanism of FIG. 18.

FIG. 21 is a side view of a tensioning mechanism according to another embodiment hereof for use in the inhaler device of FIG. 1A, wherein the tensioning mechanism is removed from the inhaler device for sake of illustration only.

FIG. 22 is a side view of the tensioning mechanism of FIG. 21.

FIG. 23A is a side view of the tensioning mechanism of FIG. 21, wherein the tensioning mechanism is in a first position.

FIG. 23B is a side view of the tensioning mechanism of FIG. 21, wherein the tensioning mechanism is in a second position.

FIG. 24 is a side view of a tensioning mechanism according to another embodiment hereof for use in the inhaler device of FIG. 1A, wherein the tensioning mechanism is removed from the inhaler device for sake of illustration only.

FIG. 25 is an opposing side view of the tensioning mechanism of FIG. 24.

FIG. 26A is a perspective view of a base of a tensioning mechanism according to another embodiment hereof for use in the inhaler device of FIG. 1A, wherein the tensioning mechanism is removed from the inhaler device for sake of illustration only.

FIG. 26B is a perspective view of a hub of the tensioning mechanism of FIG. 26A, wherein the hub is removed from the inhaler device for sake of illustration only.

FIG. 26C is a top view of the tensioning mechanism of FIG. 26A, wherein a bayonet connection thereof is in an open or unlocked state.

FIG. 26CC is a cross-sectional view of FIG. 26C.

FIG. 26D is a top view of the tensioning mechanism of FIG. 26A, wherein a bayonet connection thereof is in a closed or locked state.

FIG. 26DD is a cross-sectional view of FIG. 26D.

FIG. 26E is a perspective view of a nut of the tensioning mechanism of FIG. 26A, wherein the nut is removed from the inhaler device for sake of illustration only.

FIG. 26F is another perspective view of the hub of the tensioning mechanism of FIG. 26A, wherein the hub is removed from the inhaler device for sake of illustration only.

FIG. 26G is a perspective view of a nest fixture configured for use with the tensioning mechanism of FIG. 26A.

FIG. 26H is a perspective view of a base of the tensioning mechanism of FIG. 26A, wherein the base is removed from the inhaler device for sake of illustration only.

FIG. 26I is a perspective view of the base of FIG. 26H disposed onto the nest fixture of FIG. 26F.

FIG. 26J is a perspective view of the base of FIG. 26H disposed onto the nest fixture of FIG. 26F, with the nut disposed thereon prior to rotation of the nut by the hub.

FIG. 26K is a perspective view of the base of FIG. 26H disposed onto the nest fixture of FIG. 26F, with the nut disposed thereon after rotation of the nut by the hub.

FIG. 26L is a perspective view of the nut and the base of the tensioning mechanism of FIG. 26A at an end of device life state, wherein an audible click is output by the tensioning mechanism.

FIG. 26M is a perspective view of the nut and the base of the tensioning mechanism of FIG. 26A at a beginning of device life state.

FIG. 26N is another perspective view of the nut and the base of the tensioning mechanism of FIG. 26A at an end of device life state, wherein a lock-out mechanism of the tensioning mechanism is engaged.

FIG. 27A is a perspective view of another embodiment of a lock-out mechanism for use with the tensioning mechanism of FIGS. 26A-26N.

FIG. 27B is a perspective view of another embodiment of a lock-out mechanism for use with the tensioning mechanism of FIGS. 26A-26N.

FIG. 27C is a perspective view of another embodiment of a lock-out mechanism for use with the tensioning mechanism of FIGS. 26A-26N.

FIG. 27D is another perspective view of the lock-out mechanism of FIG. 27C.

### DETAILED DESCRIPTION OF THE INVENTION

Specific embodiments of the present invention are now described with reference to the figures, wherein like reference numbers indicate identical or functionally similar elements. The following detailed description is merely exemplary in nature and is not intended to limit the invention or the application and uses of the invention. Although the description of the invention is in the context of testing stent-graft devices, the invention may also be used to test other tubular prostheses where it is deemed useful. Furthermore, there is no intention to be bound by any expressed or implied theory presented in the preceding technical field, background, brief summary or the following detailed description.

Embodiments hereof relate to inhaler devices configured to deliver powdered medicament from at least one blister strip, and more particularly, relate to tensioning mechanisms for maintaining consistent tension in the blister strip over the lifetime of the device. The tensioning mechanisms described herein are illustrated within an inhaler device configured to simultaneously deliver powdered medicament from two blister strips but may also be utilized in an inhaler device configured to deliver powdered medicament from a single blister strip or more than two blister strips.

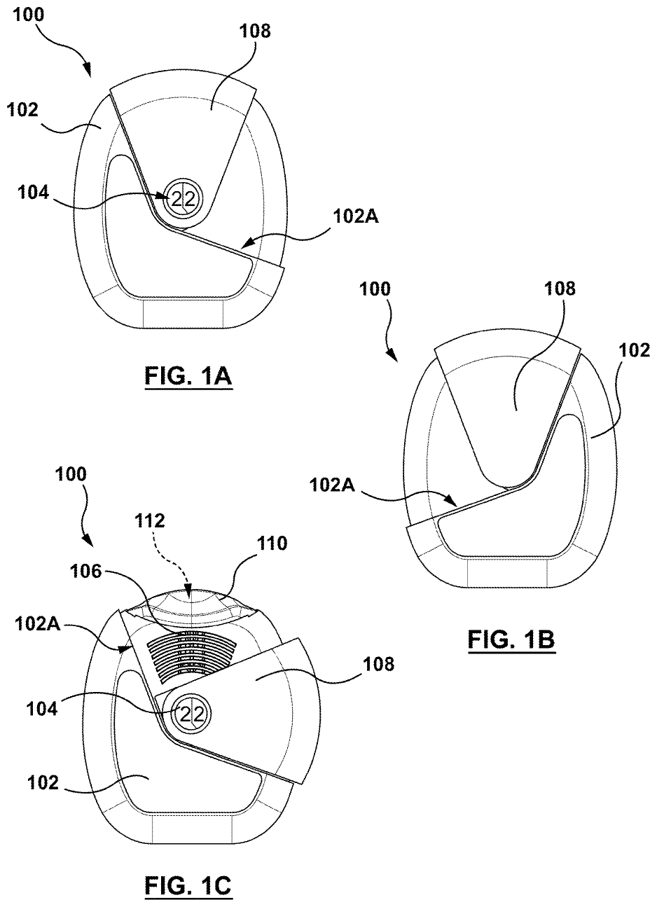

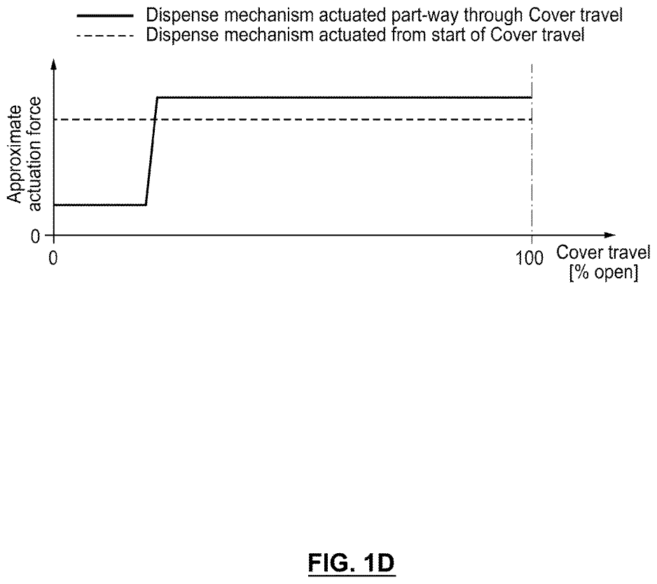

FIGS. 1A, 1B, and 1C illustrate an inhaler device 100 according to an embodiment hereof. The inhaler device 100 includes a housing 102 and a mouthpiece cover 108. The housing 102 includes a display window 104 through which a number is displayed, the number indicating the number of remaining doses of the inhaler device 100. The housing 102 also includes a plurality of openings or inlet vents 106 formed through a sidewall of the housing 102. As will be described in more detail herein, air from outside of the inhaler device 100 is drawn into the interior of the inhaler device 100 via the inlet vents 106 upon inhalation, by a user, at a mouthpiece 110. In FIGS. 1A and 1B, the mouthpiece cover 108 of the inhaler device 100 is in a closed position in which the mouthpiece cover 108 covers or extends over the mouthpiece 110. The mouthpiece cover 108 is in an open position in FIG. 1C such that the mouthpiece 110 is exposed and available to the user. The user can inhale the powdered medicament through the mouthpiece 110 only when the mouthpiece cover 108 is in the open configuration. The mouthpiece 110 includes a central outlet or opening 112 which permits delivery of the powdered medicament contained within the inhaler device 100 to the user via inhalation.

In the closed position of FIGS. 1A and 1B, the mouthpiece 110 and the inlet vents 106 are covered by the mouthpiece cover 108. When a user wishes to inhale a medicament dose from the inhaler device 100, the mouthpiece cover 108 is moved from the closed position of FIGS. 1A and 1B to the open position of FIG. 1C. In the open position, the mouthpiece cover 108 is rotated or moved relative to the housing 102 such that the mouthpiece 110 and the inlet vents 106 are fully exposed and no longer covered by any portion of the mouthpiece cover 108. Since users are instructed to cover the central opening 112 of the mouthpiece 110 with their mouth during inhalation of the powdered medicament, the mouthpiece cover 108 protects the mouthpiece 110 when the inhaler device 100 is not in use to prevent contamination of the airflow passageway of the inhaler device 100 by unwanted particles, which could otherwise have a negative impact on user experience and/or dose delivery. As will be described in more detail herein, the movement of the mouthpiece cover 108 from the closed position to the open position actuates a dispensing mechanism in the inhaler device 100 to make a medicament dose available for inhalation and further actuates a counter mechanism in the inhaler device 100 to decrease the number of remaining doses shown in the display window 104 by one unit. Thus, the mouthpiece cover 108 functions to protect the central opening 112 of the mouthpiece 110 and also operates the dispensing and counter mechanisms of the inhaler device 100. Only a single operating step, i.e., movement of the mouthpiece cover 108, is required by the user to actuate the inhaler device 100 for each dose.

In an embodiment, a mouthpiece cover 108 may be rotated between 85 and 105 degrees by the user to expose a mouthpiece 110 and inlet vents 106. In designing the inhaler device 100, it is important to ensure that a force required to actuate the mouthpiece cover 108 is sufficiently low to allow easy operation by users with a range of abilities. In general, when a distance of travel of the mouthpiece cover 108 is longer, a more favorable mechanical advantage is realized in a dispensing mechanism of the inhaler device, which results in a lower actuation force being needed to rotate the mouthpiece cover 108 and actuate the inhaler device 100 for each dose. However, from an ergonomics perspective, a shorter distance of travel of the mouthpiece cover 108 may avoid the user having to change grip during actuation. A shorter travel distance of the mouthpiece cover 108 also results in a larger area of the housing 102 for the user to grip during actuation, since there is a smaller area of the housing 102 traversed by the mouthpiece cover 108. A shorter travel of the mouthpiece cover 108 also allows more space for other features of the inhaler device 100, and/or allows a size of the inhaler device 100 to be minimized. In an embodiment, a cover travel between 90 and 100 degrees may provide an optimal balance between the above noted factors. As shown in FIGS. 1A-1C, the housing 102 includes an integral flange or step 102A formed thereon that controls or limits rotational movement of the mouthpiece cover 108 to the desired range.

The force profile over the travel of the mouthpiece cover 108 will affect the user experience and tactile feedback given by the inhaler device 100. Maintaining a relatively consistent or constant actuation force over the travel of the mouthpiece cover 108 is also desirable to avoid incorrect usage or confusion. Using the full travel of the mouthpiece cover 108 for operating the dispensing mechanism is expected to make the actuation force profile more consistent and help to mitigate the risk of misuse of the inhaler device 100. For example, in inhaler devices unlike the present invention in which a dispensing mechanism is not actuated for an initial period of the mouthpiece cover travel, an actuation force is relatively low in this initial period. At the point in which the dispensing mechanism is actuated, the actuation force of the mouthpiece cover increases. Thus in inhalers unlike the present invention, the actuation force of the mouthpiece cover significantly increases midway through the total travel of the mouthpiece cover, and a user may incorrectly perceive this change as tactile feedback suggesting that the mouthpiece cover is sufficiently open to take a dose. Stated another way, a non-constant actuation force profile may be confusing for a user in terms of the tactile feedback and could cause incorrect usage of the device. FIG. 1D illustrates an estimated actuation force profile, represented by a dashed line, for the inhaler device 100 according to embodiments herein that has the mouthpiece cover 108, as compared to an estimated actuation force profile, represented by a solid line, for an inhaler device in which the dispensing mechanism is not actuated for an initial period of the mouthpiece cover travel as explained in the prior example. When a dispensing mechanism does not actuate at the beginning of the mouthpiece travel, there is a step in the actuation force profile as represented by the solid line in FIG. 1D. Conversely, when a dispensing mechanism actuates at the beginning of the mouthpiece travel as in the inhaler device 100, then the actuation force is generally constant or consistent and the peak actuation force is lower, as represented by the dashed line in FIG. 1D.

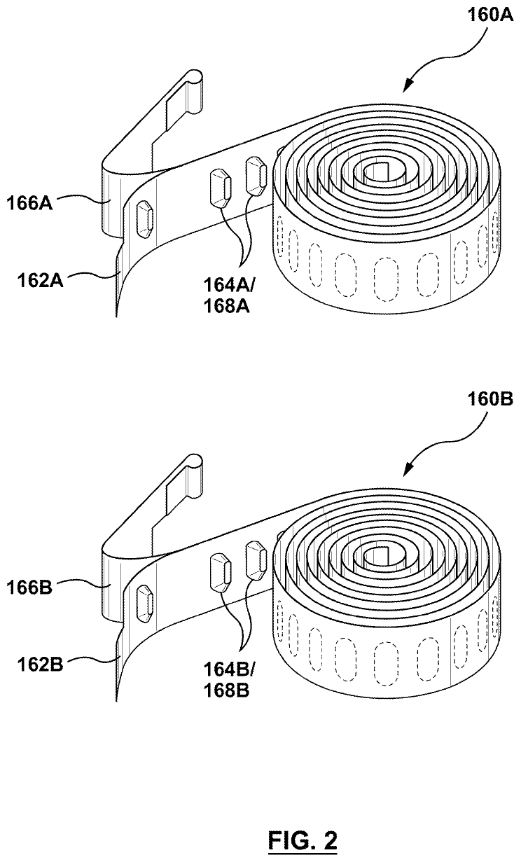

Inhaler device 100 is configured to simultaneously dispense dry powder medicaments from two blister strips. More particularly, with reference to FIG. 2, a first blister strip 160A and a second blister strip 160B are shown. Inhaler device 100 described herein is configured to simultaneously dispense medicament from each of the first and second blister strips 160A, 160B. Each blister strip 160A, 160B includes a bottom sheet 162A, 162B, respectively, which defines a series or plurality of individual blisters or pockets 164A, 164B thereon. Each pocket 164A, 164B is configured to contain a dose or portion thereof of dry powder or powdered medicaments 168A, 168B to be inhaled by a user. In an embodiment, the powdered medicament 168A is a different medicament than the powdered medicament 168B so that the inhaler device 100 is configured to simultaneously deliver two different powdered medicaments to the user. A top sheet 166A, 166B is hermetically bonded or sealed to the bottom sheet 162A, 162B, respectively, to close the pockets 164A, 164B, respectively and function as a lid for the pockets 164A, 164B to retain the powdered medicaments 168A, 168B therein. The hermetic sealing of the top sheets 166A, 166B is such that the bottom sheets 162A, 162B and the top sheets 166A, 166B are able to be peeled apart to open or uncover the pockets 164A, 164B for access to the powdered medicaments 168A, 168B therein. Each of the first and second blister strips 160A, 160B is sufficiently flexible to be wound into a roll.

As will be described in more detail herein, when a dispensing mechanism of the inhaler device 100 is actuated via movement of the mouthpiece cover 108, the top sheets 166A, 166B of the blister strips 160A, 160B, respectively, are peeled away from the bottom sheets 162A, 162B of the blister strips 160A, 160B, respectively, to open or uncover a pocket 164A, 164B of each blister strip and thereby expose the powdered medicaments 168A, 168B disposed therein. Upon inhalation via the mouthpiece 110, the user simultaneously inhales the powdered medicaments 168A, 168B from the opened pockets 164A, 164B of the blister strips 160A, 160B, respectively. The user thus receives a fixed metered dose of medicament powder of which the different medicament powders from the opened pockets 164A, 164B of the blister strips 160A, 160B, respectively, make up respective dose portions. Each blister strip 160A, 160B may be of the same size and/or contain the same dose amount (e.g., volume or mass) of powdered medicament or may be of different sizes and/or contain different dose amounts of powdered medicament.

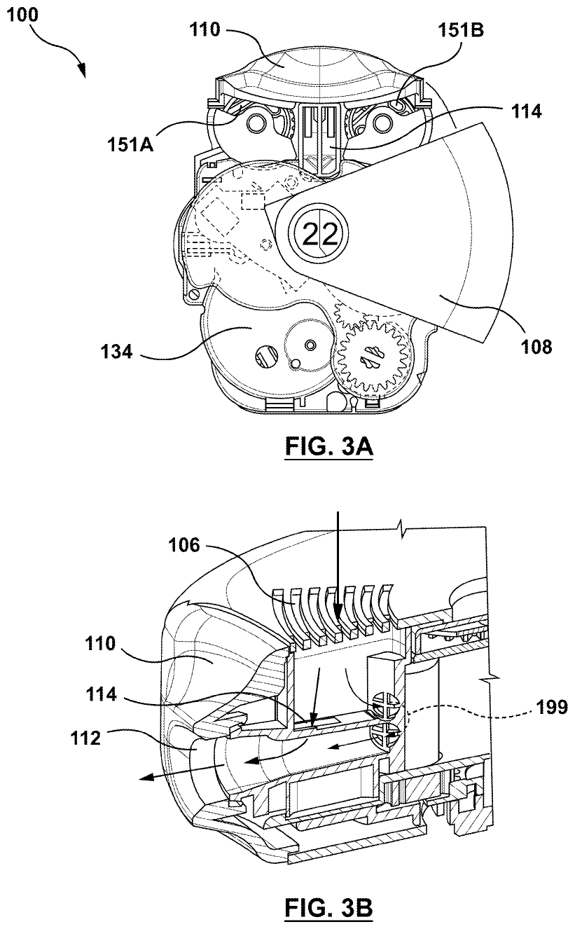

FIG. 3A is a front view of the inhaler device 100 with the mouthpiece cover 108 in the open position and the housing 102 removed for sake of illustration only. The inhaler device 100 includes a manifold 114 for directing airflow therethrough to entrain and deliver the powdered medicaments 168A, 168B from the blister strips 160A, 160B, respectively, to the user via the mouthpiece 110. The manifold 114 is in fluid communication with the mouthpiece 110 such that the powdered medicaments 168A, 168B may be delivered to the user through the central opening 112 of the mouthpiece 110. In addition to the manifold 114, the inhaler device 100 also includes a dispensing subassembly or mechanism 120, a counter subassembly or mechanism 134, and tensioning subassemblies or mechanisms 151A, 151B. When assembled, each of the manifold 114, the dispensing subassembly 120, the counter subassembly 134, and the tensioning mechanisms 151A, 151B reside or are disposed within the housing 102.

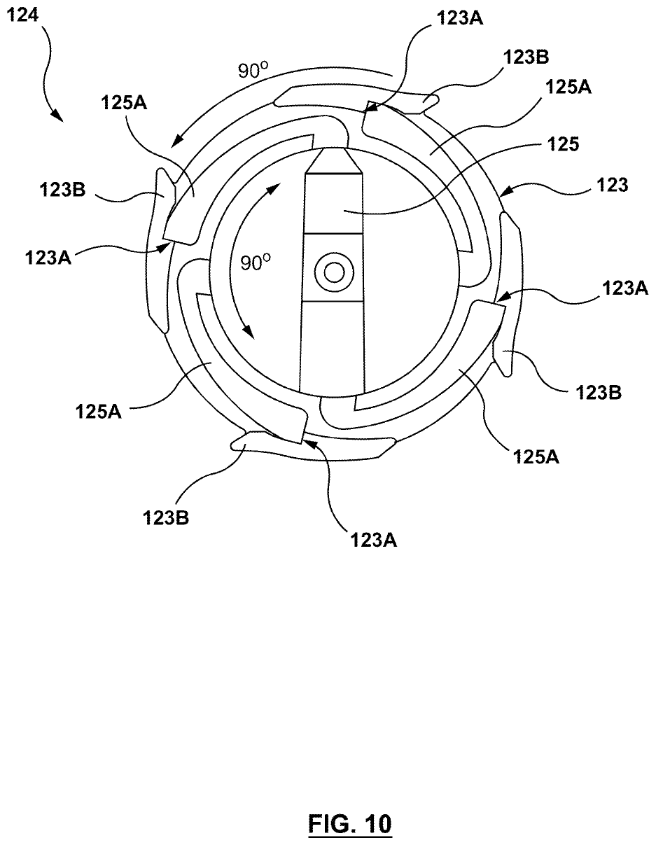

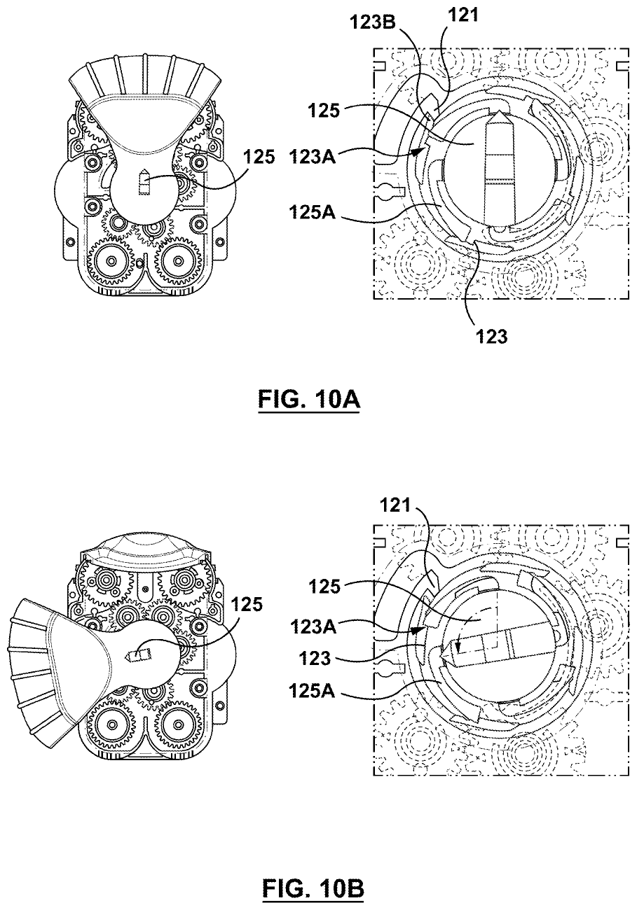

The operation of the manifold 114 is introduced herein with reference to FIG. 3B. The manifold 114 defines the air pathway through the inhaler device 100. The manifold 114 fluidly connects the mouthpiece 110 to the first and second blister strips 160A, 160B. FIG. 3B is a sectional view taken through the manifold 114 to illustrate an airflow path therethrough for entrainment of medicament 168B of the second blister strip 160B. As explained with respect to FIGS. 10-14, the manifold 114 also defines an airflow path therethrough for entrainment of medicament 168A of the first blister strip 160A.

In use, the mouthpiece cover 108 is rotated by the user to expose the mouthpiece 110 and the inlet vents 106. Internally, within the inhaler device 100, rotating the mouthpiece cover 108 exposes the powdered medicaments 168A, 168B within a pocket 164A, 164B, respectively, of each of first and second blister strips 160A, 160B. To access the powdered medicaments 168A, 168B within the opened pockets 164A, 164B, the user breathes in or inhales through the mouthpiece 110. By covering the central opening 112 of the mouthpiece 110 with their mouth and inhaling, the user creates a pressure differential between the inlet vents 106 and the central opening 112 and causes air to travel through the manifold 114. The pressure differential causes external air (i.e., air from outside of the inhaler device 100) to enter the inhaler device 100 through the inlet vents 106, pass through the opened pockets 164A, 164B, and exit the inhaler device 100 through the central opening 112. The airflow pathways defined by the manifold 114 are designed such that when the user inhales, the exposed powdered medicaments 168A, 168B within the opened pockets 164A, 164B are picked up by the airstreams and delivered to the user as an orally inhaled combined medicament dose. As such, the user can simultaneously inhale one dose portion from each blister strip 160A, 160B.

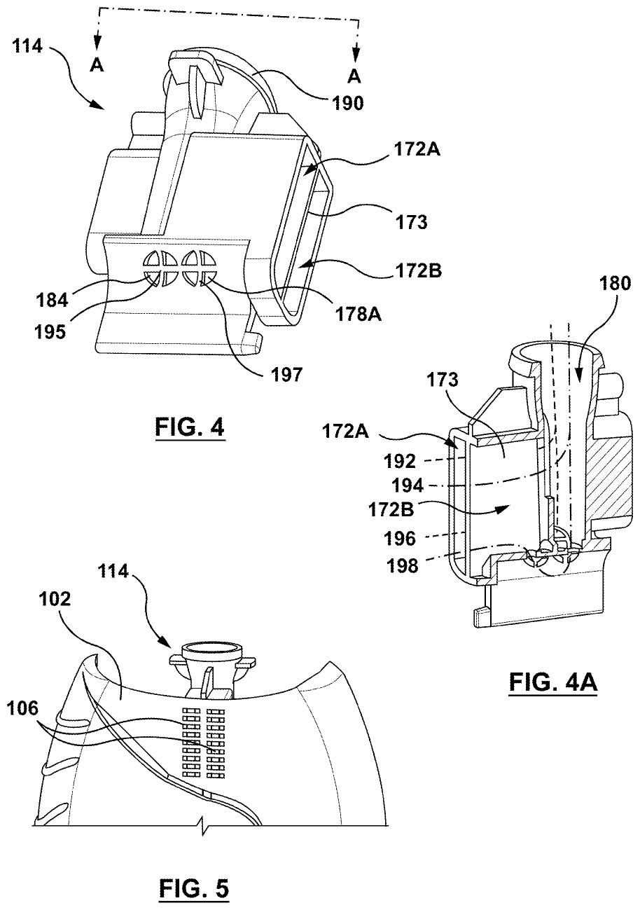

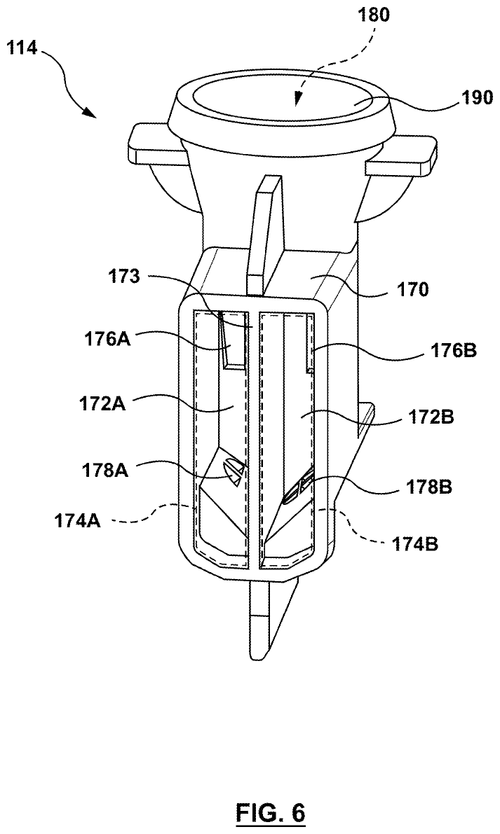

The manifold 114 is configured to direct the inhalation airflow in a variety of ways to achieve airflow properties that are advantageous for effective delivery of the powdered medicaments. More particularly, as shown in FIG. 3B, the geometry of manifold 114 causes a portion of the inhalation airflow to enter and exit an opened pocket 164B of the second blister strip 160B (labeled with a dotted line 199 in FIG. 3B), while another portion of the inhalation airflow travels through a diversion or hole in the manifold 114 (which is described in more detail in FIGS. 4-8). The portion of inhalation airflow through the opened pocket 164B results in entrainment of the dose of powdered medicament 168B in the airflow, and the portion of inhalation airflow through the diversion in the manifold 114 intersects the entrained airflow portion to break up the powdered medicament therein before exiting the manifold 114.

Turning to FIGS. 4-8, the manifold 114 is described in more detail. The manifold 114 is configured for simultaneous delivery of powdered medicament 168A, 168B from respective open blister pockets 164A, 164B, of each of the first blister strip 160A and the second blister strip 160B, respectively. The manifold 114 includes a body 170 that defines a first space or atrium 172A, a second space or atrium 172B, and a stack 180. As will be explained in more detail herein, the separated distinct compartments or spaces of the first atrium 172A, the second atrium 172B, and the stack 180 split up, divide, or otherwise separate an inhalation airflow, which is drawn into the manifold 114 by a user, into multiple airflow paths through the body 170 of the manifold 114. More specifically, when an inhalation force is applied through the central opening 112 of the mouthpiece 110, an inhalation airflow is drawn into the first and second atriums 172A, 172B of the manifold 114 via the inlet vents 106 of the inhaler device 100. The first and second atriums 172A, 172B are disposed adjacent to, or are in a juxtaposed relation with, the inlet vents 106. Once the inhalation airflow enters into the manifold 1114, it splits or divides into four airflow paths as it travels through the body 170 of the manifold, namely, a first diversion airflow path 192, a second diversion airflow path 194, a first entrainment airflow path 196, and a second entrainment airflow path 198. Stated another way, each of the first diversion airflow path 192, the second diversion airflow path 194, the first entrainment airflow path 196, and the second entrainment airflow path 198 are respective airflow portions of the inhalation airflow which is drawn into the manifold 114.

In this embodiment, the first atrium 172A and the second atrium 172B are disposed laterally adjacent to each other, or side-by-side, on a single side of the body 170 of the manifold 114. The first atrium 172A and the second atrium 172B are separated from each other by a divider wall 173 such that the first atrium 172A is not in fluid communication with the second atrium 172B. The first atrium 172A includes a single atrium inlet 174A, and the second atrium 172B includes a single atrium inlet 174B. The atrium inlets 174A, 174B are separate from each other and may also be considered the inlets of the manifold 114. As such, the manifold 114 includes two inlets, the atrium inlet 174A leading or entering into the first atrium 172A and the atrium inlet 174B leading or entering into the second atrium 172B.

The first atrium 172A includes a first atrium outlet 176A and a second atrium outlet 178A, and the second atrium 172B includes a first atrium outlet 176B and a second atrium outlet 178B. As will be explained in more detail herein, the first atrium outlet 176A, 176B of each of the first and second atriums 172A, 172B, respectively, directs or guides flow directly into the stack 180 and the second atrium outlet 178A, 178B of each of the first and second atriums 172A, 172B, respectively, directs or guides flow into an open pocket 164A, 164B, respectively, of the first and second blister strips 160A, 160B, respectively. In an embodiment, the profile or shape of the first atrium outlet 176A, 176B is substantially rectangular or oblong. However, the profile or shape of the first atrium outlets 176A, 176B is not limited to the shape depicted herein and may alternatively be circular, triangular, or any other shape deemed suitable for the purposes described herein. Similarly, the profile or shape of the second atrium outlets 178A, 178B is substantially circular and includes a grill or cross-piece 197 (see FIG. 4) spanning thereover to encourage increased turbulence in the airflow. However, the profile or shape of the second atrium outlets 178A, 178B is not limited to the shape depicted herein and may alternatively be rectangular, oblong, oval, triangular, or any other shape deemed suitable for the purposes described herein and may or may not include a grill spanning thereover.

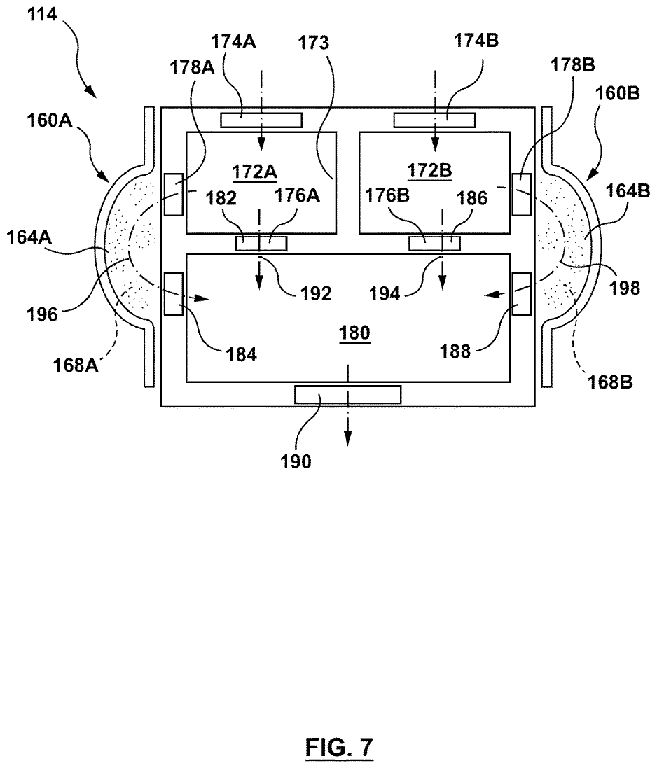

As best shown on FIG. 7, the stack 180 is in fluid communication with each of the first atrium 172A, the second atrium 172B, the open pocket 164A of the first blister strip 160A, and the second open pocket 164B of the second blister strip 160B. The stack 180 has four inlets, namely, a first stack inlet 182, a second stack inlet 184, a third stack inlet 186, and a fourth stack inlet 188. The first stack inlet 182 is aligned with the first atrium outlet 176A of the first atrium 172A, such that the stack 180 and the first atrium 172A are in fluid communication with each other. The second stack inlet 184 is aligned with the open pocket 164A of the first blister strip 160A, such that the second stack inlet 184 is further in fluid communication with the second atrium outlet 178A of the first atrium 172A via the open pocket 164A. The third stack inlet 186 is in fluid communication with the second atrium outlet 176B of the second atrium 172B, such that the stack 180 and the second atrium 172B are in fluid communication with each other. The fourth stack inlet 188 is in fluid communication with the open pocket 164B of the second blister strip 160B, such that the fourth stack inlet 188 is further in fluid communication with the second atrium outlet 178B of the second atrium 172B via the open pocket 164B.

The profile or shape of each of the first stack inlet 182 and the third stack inlet 186 is substantially rectangular or oblong. However, the profile or shape of the first and third stack inlets 182, 186 is not limited to the shape depicted herein and may alternatively be circular, triangular, or any other shape deemed suitable for the purposes described herein. Similarly, the profile or shape of the second stack inlet 184 and the fourth stack inlet 188 is substantially circular and includes a grill or cross-piece 195 (see FIG. 4) spanning thereover to encourage increased turbulence in the airflow. However, the profile or shape of the second and fourth stack inlets 184, 188 is not limited to the shape depicted herein and may alternatively be rectangular, oblong, oval, triangular, or any other shape deemed suitable for the purposes described herein and may or may not include a grill spanning thereover.

The stack 180 is a single stack with a single stack outlet 190. Thus in the present embodiment, the stack 180 has only one stack outlet 190. The stack outlet 190 may also be considered the outlet of the manifold 114. As such, the manifold 114 includes only one outlet. A shape or profile of the stack outlet 190 is oval. However, the profile or shape of the stack outlet 190 is not limited to the shape depicted herein and may alternatively be circular, rectangular, oblong, triangular, or any other shape deemed suitable for the purposes described herein. When the manifold 114 is assembled into the inhaler device 100, the stack outlet 190 is aligned and in fluid communication with the central opening 112 of the mouthpiece 110.

The second atrium outlet 178A of the first atrium 172A is in fluid communication with the second stack inlet 184 to define the first entrainment airflow path 196 associated with an open blister pocket 164A of the first blister strip 160A. As the airstream flows through the open blister pocket 164A, it picks up the powdered medicament 168A disposed within the open blister pocket 164A. Thus, by passing through the open blister pocket 164A, the powdered medicament 168A is drawn in and transported by the airstream into the stack 180. After entrainment, the airstream is laden with the powdered medicament 168A.

Similarly, the second atrium outlet 178B of the second atrium 172B is in fluid communication with the fourth stack inlet 188 to define the second entrainment airflow path 198 associated with an open blister pocket 164B of the second blister strip 160B. As the airstream flows through the open blister pocket 164B, it picks up the powdered medicament 168B disposed within the open blister pocket 164B. Thus, by passing through the open blister pocket 164B, the powdered medicament 168B is drawn in and transported by the airstream into the stack 180. After entrainment, the airstream is laden with the powdered medicament 168B.

The first atrium outlet 176A of the first atrium 172A is in fluid communication with the first stack inlet 182 to define the first diversion airflow path 192 of the manifold 114. Similarly, the first atrium outlet 176B of the second atrium 172B is in fluid communication with the third stack inlet 186 to define the second diversion airflow path 194 of the manifold 114. Each of the diversion airflow paths 192, 194 provides a lower resistance pathway for air to flow from outside the inhaler device 100 to the patient's mouth compared to the first and second entrainment airflow paths 196, 198. As a result, the overall airflow resistance of the inhaler device 100 is lowered so that higher overall flow rates can be achieved for the same inhalation pressure. In addition, the diversion airflow paths 192, 194 provide de-agglomeration of the powdered medicaments 168A, 168B before they exit the manifold 114. Each of the first and second diversion airflow paths 192, 194 is configured to disrupt each of the first and second entrainment airflow paths 196, 198 and break up medicament carried thereby. More particularly, the first diversion airflow path 192 is directed into the path of the first entrainment airflow path 196, which is at a different angle from the first diversion airflow path 192. A region of higher shear is created at the point of intersection between the first diversion airflow path 192 and the first entrainment airflow path 196, improving de-agglomeration of the powdered medicament 168A before it exits the manifold 114. Similarly, the second diversion airflow path 194 is directed into the path of the second entrainment airflow path 198, which is at a different angle from the second diversion airflow path 194. A region of higher shear is created at the point of intersection between the second diversion airflow path 194 and the second entrainment airflow path 198, improving de-agglomeration of the powdered medicament 168B before it exits the manifold 114. The first diversion airflow path 192, the second diversion airflow path 194, the first entrainment airflow path 196, and the second entrainment airflow path 198 combine or mix together within the stack 180 before exiting the manifold 114.

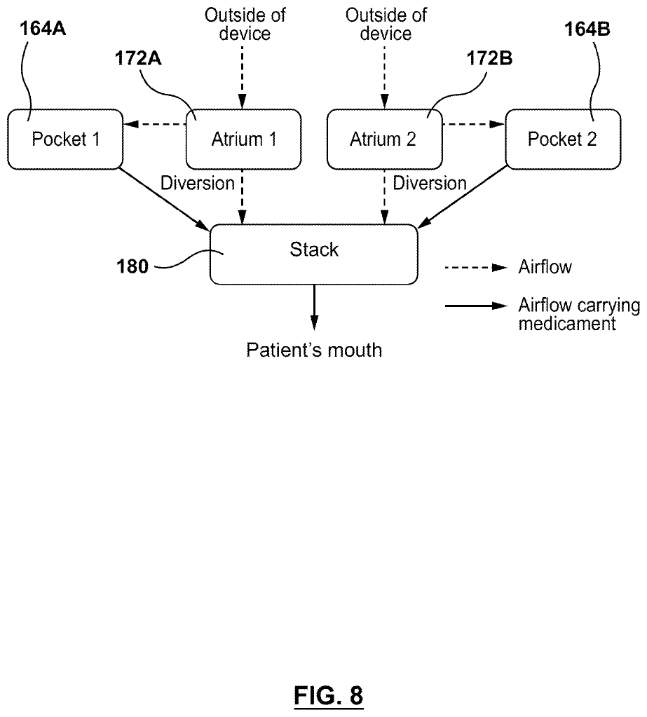

As represented in the flowchart of FIG. 8, the inhalation airstream drawn from outside the inhaler device 100 is divided between the two inlets of the manifold 114, namely the first atrium inlet 174A and the second atrium inlet 174B. The inhalation airstream drawn from outside the inhaler device thus simultaneously enters each of the first atrium 172A and the second atrium 172B. A first portion of the inhalation airstream entering the first atrium 172A flows into the open blister pocket 164A and a second portion of the inhalation airstream entering the first atrium 172A directly flows into the stack 180. The first portion of the inhalation airstream within the open blister pocket 164A picks up or entrains powdered medicament 168A disposed within the open blister pocket 164A, and then continues into the stack 180. Within the stack 180, the second portion of the inhalation airstream from the first atrium 172A breaks up or de-agglomerates the powdered medicament 168A entrained within the first portion of the inhalation airstream. Similarly, at the same time, a first portion of the inhalation airstream entering the second atrium 172B flows into the open blister pocket 164B and a second portion of the inhalation airstream entering the second atrium 172B flows into the stack 180. The first portion of the inhalation airstream within the open blister pocket 164B picks up or entrains powdered medicament 168B disposed within the open blister pocket 164B, and then continues into the stack 180. Within the stack 180, the second portion of the inhalation airstream from the second atrium 170B breaks up or de-agglomerates the powdered medicament 168B entrained within the first portion of the inhalation airstream. Within the stack 180, all portions of the airstream mix together before exiting the manifold 114 towards a patient's mouth, with the combined airstream including both medicament 168A from the first blister strip 160A and medicament 168B from the second blister strip 160B. Airflow through the first atrium 172A and the open blister pocket 164A is concurrent to airflow through the second atrium 172B and the open blister pocket 164B.

Due to the fact that the first and second atriums 172A, 172B are separate and distinct compartments with the divider wall 173 extending therebetween, the inhalation airstream entering into the manifold 114 is directed towards the outlets of each atrium. Directing the separated inhalation airstream into the open blister pockets 164A, 164B in this way reduces turbulent energy in the airflow at this stage, and therefore reduces overall airflow resistance of the inhaler device 100. Overall airflow resistance allows patients to achieve higher flow rates for the same inhalation pressure, which may improve efficacy of drug delivery.

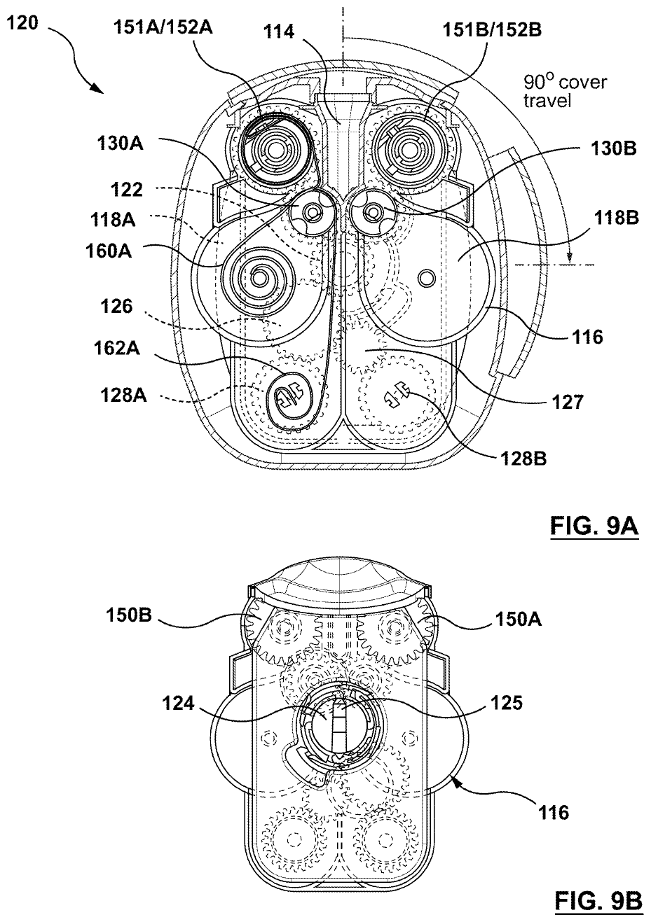

Turning now to FIGS. 9A and 9B, the dispensing subassembly 120 of the inhaler device 100 is described in more detail. The dispensing subassembly 120 is configured to advance each blister strip 160A, 160B and open a pocket 164A, 164B thereof each time the mouthpiece cover 108 is fully opened by the user. The first and second blister strips 160A, 160B are disposed within first and second compartments 118A, 118B within the housing 102. More particularly, compartments 118A, 118B are formed via an internal chassis 116 disposed within the housing 102. Via the dispensing subassembly 120, successive pockets 164A, 164B of each blister strip 160A, 160B are guided towards the manifold 114 which is disposed along a centerline, or disposed approximately along the centerline, of the inhaler device 100. When positioned at the manifold 114, a pocket 164A, 164B of each blister strip 160A, 160B has been opened and the powdered medicaments 168A, 168B within the opened pocket of each blister strip 160A, 160B is available for inhalation. The empty bottom sheets 162A, 162B and the top sheets 166A, 166B of the blister strips 160A, 160B are coiled up by the dispensing subassembly 120 as described herein. FIG. 9A is a front view of the inhaler device 100 with the mouthpiece cover 108 in the open position and a front half or portion of the housing 102 of the inhaler device 100 is removed for sake of illustration only. FIG. 9B is a rear view of the inhaler device 100 with the mouthpiece cover 108 and the housing 102 removed for sake of illustration only.

The dispensing subassembly 120 includes a central driver gear 122, a ratchet mechanism 124, a first idler or intermediate gear 126, a second idler or intermediate gear 127, first and second bottom sheet take-up gears 128A, 128B, first and second index gears 130A, 130B, and first and second top sheet take-up gears 150A, 150B. The first bottom sheet take-up gear 128A, the first index gear 130A, and the first top sheet take-up gear 150A are associated with advancement of the first blister strip 160A, while the second bottom sheet take-up gear 128B, the second index gear 130B, and the second top sheet take-up gear 150B are associated with advancement of the second blister strip 160B.

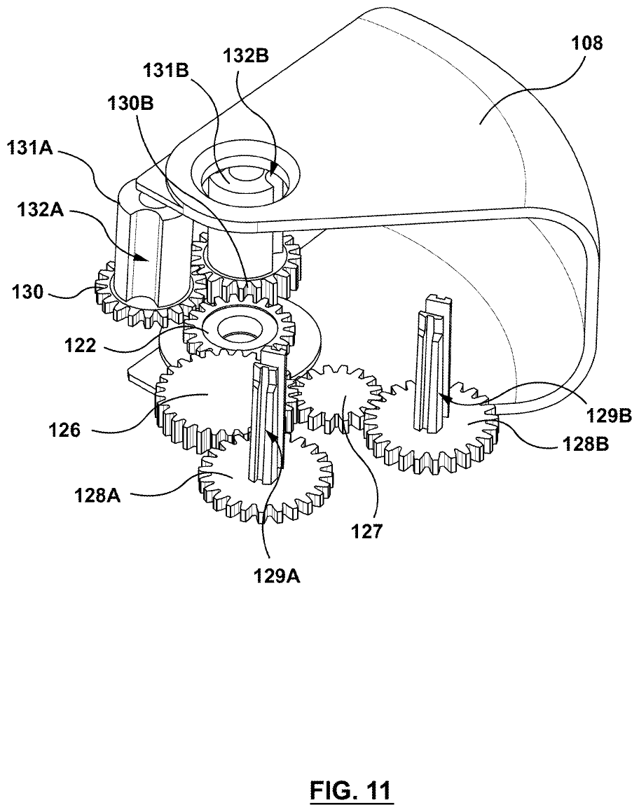

The first and second index gears 130A, 130B are attached to or integrally formed with first and second index spools 131A, 131B, respectively, as shown in FIG. 11. Each of first and second index spools 131A, 131B include a pair of recesses 132A, 132B, respectively, thereon. Each recess of the pair of recesses 132A, 132B is configured to receive a pocket 164A, 164B, respectively, of the blister strips 160A, 160B. Via rotation of the first and second index gears 130A, 130B, the first and second index spools 131A, 131B rotate and operate to move a recess 132A, 132B, respectively, adjacent to or in juxtaposition with the manifold 114. As the blister strips 160A, 160B are advanced by the index spools 131A, 131B, the top sheets 166A, 166B of the blister strips 160A, 160B are peeled away from the bottom sheets 162A, 162B of the blister strips such that a pocket 164A, 164B thereof adjacent to the manifold 114 is opened and the powdered medicaments 168A, 168B therein is available for entrainment. More particularly, in order to expose the powdered medicaments 168A, 168B for each dose, the top sheets 166A, 166B are peeled away from the bottom sheets 162A, 162B, respectively, such that a pocket 164A, 164B in each bottom sheet 162A, 162B is opened or uncovered. Uncovering or opening of a pocket 164A, 164B is achieved by relative rotation between the index spools 131A, 131B and the top sheet take-up gears 150A, 150B. The index spools 131A, 131B essentially grip the bottom sheets 162A, 162B, respectively, and the top sheet take-up gears 150A, 150B, respectively, essentially grip the top sheets 166A, 166B. When the index spools 131A, 131B and the top sheet take-up gears 150A, 150B rotate relative to each other, the bottom sheets 162A, 162B and the top sheets 166A, 166B are peeled apart from each other. As will be described in more detail herein, the index spools 131A, 131B and the top sheet take-up gears 150A, 150B are driven via gearing to rotate in opposite directions, such that each top sheet 166A, 166B is peeled away from its respective bottom sheet 162A, 162B as the gears are driven. The dispensing subassembly 120 thus causes the respective leading pocket 164A, 164B to be opened and positioned into fluid communication with manifold 114 so that the powdered medicaments 168A, 168B of the opened pockets 164A, 164B are available for inhalation.

The first and second bottom sheet take-up gears 128A, 128B operate to wind up the empty bottom sheets 162A, 162B, respectively, of the blister strips 160A, 160B. As shown in FIG. 11, the first and second bottom sheet take-up gears 128A, 128B are attached to or integrally formed with spindles 129A, 129B, respectively. Via rotation of the first and second bottom sheet take-up gears 128A, 128B, the first and second spindles 129A, 129B rotate and operate to take-up the bottom sheets 162A, 162B as the inhaler device 100 is operated. The end of each bottom sheet 162A, 162B is anchored to the first and second bottom sheet take-up gears 128A, 128A such that progressive rotation of the first and second bottom sheet take-up gears 128A, 128B results in the bottom sheets 162A, 162B being wound therearound into a tight coil.

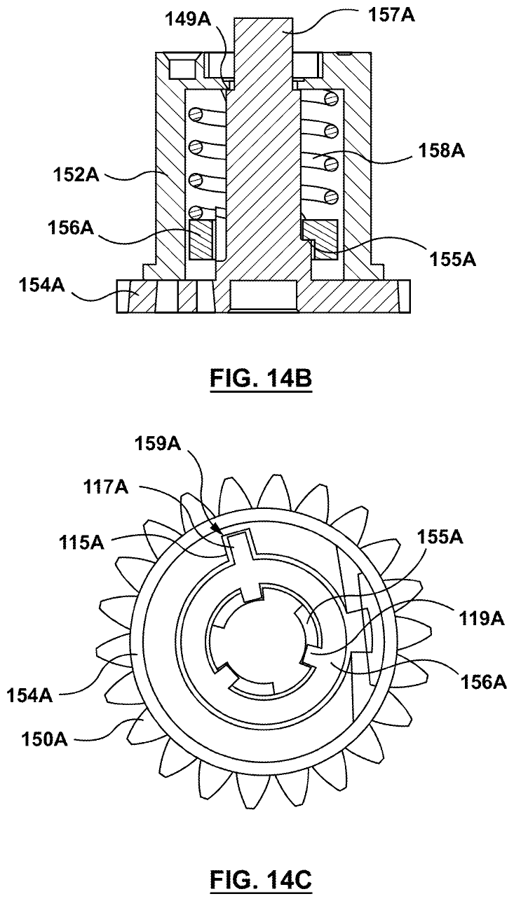

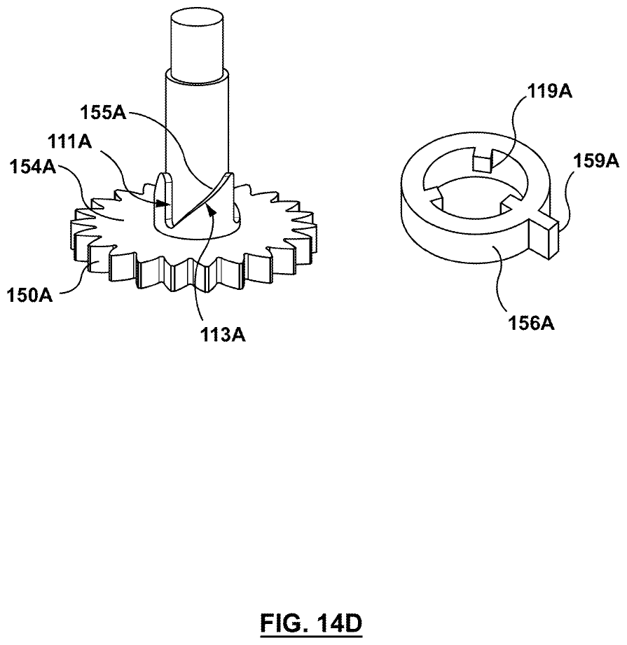

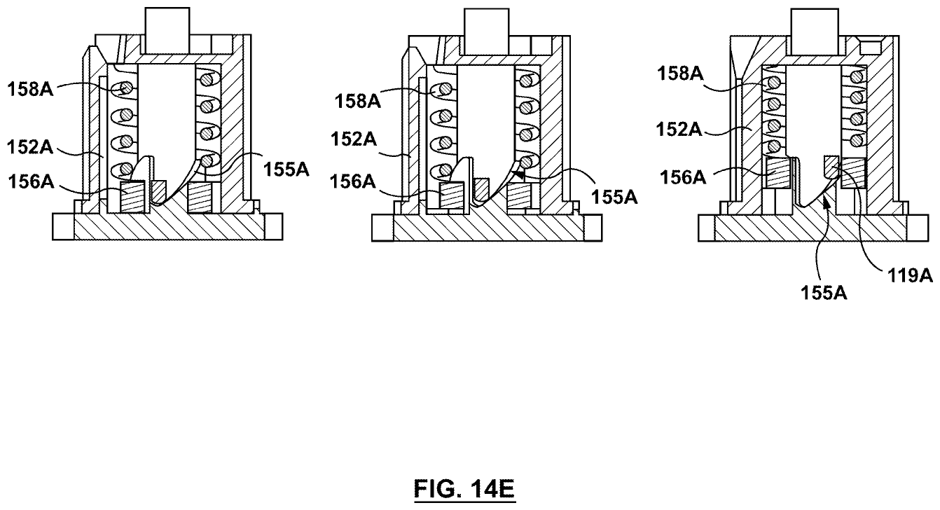

The first and second top sheet take-up gears 150A, 150B operate to wind up the top sheets 166A, 166B, respectively, of the blister strips 160A, 160B. As will be described in more detail herein with respect to FIGS. 14A-14E, the first and second top sheet take-up gears 150A, 150B are coupled to take-up hubs 152A, 152B, respectively. Via rotation of the first and second top sheet take-up gears 150A, 150B, the first and second take-up hubs 152A, 152B rotate and operate to take or wind up the top sheets 166A, 166B as the inhaler device 100 is operated. The end of each top sheet 166A, 166B is anchored to the first and second take-up hubs 152A, 152B such that progressive rotation of the first and second top sheet take-up gears 150A, 150B results in the top sheets 166A, 166B being wound therearound into a tight coil.

The central driver gear 122 of the dispensing subassembly 120 is attached to the mouthpiece cover 108 via the ratchet mechanism 124. The ratchet mechanism 124 is shown removed from the inhaler device 100 in FIG. 10. The ratchet mechanism 124 includes a ratchet 125 which is attached to the mouthpiece cover 108 and a ratchet gear 123 that is formed with or attached to the central driver gear 122. When the mouthpiece cover 108 is opened, the ratchet 125 is driven in the second opposing direction with the mouthpiece cover 108. The ratchet 125 in turn drives the ratchet gear 123 in the second opposing direction to advance or actuate the dispensing subassembly 120. When the mouthpiece cover 108 is returned to its closed position, the dispensing subassembly 120 is not advanced or actuated and remains stationary. An opening motion of the mouthpiece cover 108 is therefore transmitted to the central driver gear 122 but a closing motion of the mouthpiece cover 108 is not transmitted to the central driver gear 122.

More particularly, the ratchet gear 123 includes a plurality of circumferentially-spaced apart interior stop faces 123A and exterior stop faces 123B around an outer perimeter or edge thereof. The ratchet 125 includes a plurality of flexible ratchet arms 125A, which are configured to interact with the circumferentially-spaced apart interior stop faces 123A of the ratchet gear 123. The ratchet 125 rotates in a first direction with the mouthpiece cover 108 when the mouthpiece cover 108 is moved from a closed first position to an open second position. When rotating in the first direction, the ratchet arms 125A engage and drive the circumferentially-spaced apart interior stop faces 123A of the ratchet gear 123 as shown in FIG. 10B such that torque is transmitted to the central driver gear 122. Since the ratchet gear 123 is attached to or formed with the central driver gear 122, the central driver gear 122 rotates in the first direction concurrently with the mouthpiece cover 108. As described above, the movement of the mouthpiece cover 108 to the second position results in the opening and positioning of a pocket 164A, 164B of each blister strip 160A, 160B for subsequent simultaneous inhalation of the powdered medicaments 168A, 168B by the patient.

When the mouthpiece cover 108 is returned to its closed first position, however, the reverse rotation of the ratchet 125 is not transmitted to the central driver gear 122 because the ratchet arms 125A do not interact with the interior stop faces 123A of the ratchet gear 123. More particularly, when the ratchet 125 is rotated in the second opposing direction (i.e., counter clockwise), the ratchet arms 125A deflect radially inwards and no significant torque is transferred to the ratchet gear 123 and the central driver gear 122. Frictional drag between the ratchet 125 and ratchet gear 123 may tend to briefly drag the ratchet gear 123 in the second opposing direction (i.e., counter clockwise), but back-winding is prevented by interaction between one of the exterior stop faces 123B of the ratchet gear 123 and a detent arm 121 in a retainer plate of the inhaler device 100, as shown in FIG. 10A.

In an embodiment, a detent (not shown) may be disposed between an inner surface of the mouthpiece cover 108 and an outer surface the housing 102. The detent may be a mating protrusion and groove that is configured to temporarily resist or prevent the movement of the mouthpiece cover 108 relative to the housing 102 until a user applies a force to the mouthpiece cover 108 (i.e., when opening the mouthpiece cover 108) to release the detent by causing one of the mating features of the detent to exit or move beyond the other of the mating features of the detent. For example, a protrusion, bump or other raised structure may be formed on the outer surface of the housing 102 and a mating groove, dimple or other indentation structure may be formed on the inner surface of the mouthpiece cover 108. Alternatively, the protrusion, bump or other raised structure may be formed on the inner surface of the mouthpiece cover 108 and a mating groove, dimple or other indentation structure may be formed on the outer surface of the housing 102. The groove is configured to receive the indentation when the mouthpiece cover 108 is in the closed position of FIG. 1A. When a user applies a force sufficient to overcome the friction between the mating protrusion and groove, the mouthpiece cover 108 begins to open and move away from the closed position of FIG. 1A. The detent is configured to prevent or deter unintentional openings of the mouthpiece cover 108. In addition, the detent is configured to account for rotational clearances or tolerances within the ratchet mechanism 124 such that the dispensing and counter mechanisms in the inhaler device 100 are actuated upon the first or initial movement of the mouthpiece cover 108 away from the closed position towards the open position.

As best shown on FIG. 11, the central driver gear 122 directly or indirectly drives the remaining gears of the dispensing subassembly 120. This gear train arrangement provides for incremental indexing or advancement of the blister strips 160A, 160B via the first and second index gears 130A, 130B and also provides for winding of the top and bottom sheets of the blister strips 160A, 160B (via the top sheet take-up gears 150A, 150B and the bottom sheet take-up gears 128A, 128B, respectively) due to rotational movement in the first direction of the mouthpiece cover 108 from its closed first position to its open second position. As the central driver gear 122 rotates in the first direction with the mouthpiece cover 108, the central driver gear 122 mates with or directly drives the second index gear 130B to rotate in a second opposing direction. The second spool 131B, and the second blister strip 160B advanced thereby, thus also rotates in the second opposing direction. As the second index gear 130B rotates in the second opposing direction, the second index gear 130B mates with or directly drives the first index gear 130A to rotate in the first direction. The first index spool 131A, and the first blister strip 160A advanced thereby, thus also rotates in the first direction.

In an embodiment, a first direction is clockwise, and a second opposing direction is counter-clockwise. In the depicted embodiment, the central driver gear 122 rotates in a clockwise direction when the mouthpiece cover 108 is opened. As such, the second index gear 130B (along with the second spool 131B and the second blister strip 160B) rotate in a counter-clockwise direction and the first index gear 130A (along with the first index spool 131A and the first blister strip 160A) rotate in a clockwise direction. As will be appreciated by one of ordinary skill in the art, however, the first direction may alternatively be counter-clockwise and the second opposing direction may be clockwise so long as the gear train formed by the dispensing subassembly 120 causes the first blister strip 160A to move or advance in an opposite direction from the second blister strip 160B. In addition, as will be appreciated by one of ordinary skill in the art, the gear train formed by the dispensing subassembly 120 may include one or more idler gears (not shown) which alter the sequence of rotation between the central driver gear 122, the second index gear 130B and the first index gear 130A. The presence of such idler gears do not impact the overall function of the gear train, so long as the gear train causes the first blister strip 160A to move or advance in an opposite direction from the second blister strip 160B. For example, an idler gear (not shown) may be disposed between the central driver gear 122 and the first index gear 130A and the central driver gear 122 (rotating in the first direction) directly drives the idler gear to rotate in the second opposing direction. The idler gear may be positioned to directly drive the first index gear 130A in the first direction, and the first index gear 130A directly drives the second index gear 130B in the second opposing direction.

In order to keep tension applied to the blister strips 160A, 160B, the first and second bottom sheet take-up gears 128A, 128B rotate concurrently with and in the same direction as the first and second index gears 130A, 130B, respectively, and the first and second top sheet take-up gears 150A, 150B rotate concurrently with and in the opposite direction as the first and second index gears 130A, 130B, respectively. As the central driver gear 122 rotates in the first direction, the central driver gear 122 mates with or directly drives the first idler gear 126 to rotate in the second opposing direction and the first idler gear 126 mates with or directly drives the first bottom sheet take-up gear 128A to rotate in the first direction. The first spindle 129A, and the bottom sheet 162A wound thereby, thus also rotate in the first direction to wind or take-up the empty bottom sheet 162A of the first blister strip 160A as the first blister strip 160A is advanced by the dispensing subassembly 120. In addition, the first top sheet take-up gear 150A interacts with or is driven by the first index gear 130A. Since the first index gear 130A rotates in the first direction, the first top sheet take-up gear 150A is driven to rotate in the second opposing direction to wind up the top sheet 166A of the first blister strip 160A.

As the central driver gear 122 rotates in the first direction, the first idler gear 126 is driven to rotate in the second opposing direction as described above and the first idler gear 126 mates with or directly drives the second idler gear 127 to rotate in the first direction. The second idler gear 127 mates with or directly drives the second bottom sheet take-up gear 128B to rotate in the second direction. The second spindles 129B, and the bottom sheet 162B wound thereby, thus also rotate in the second direction to wind or take-up the empty bottom sheet 162B of the second blister strip 160B as the second blister strip 160B is advanced by the dispensing subassembly 120. In addition, the second top sheet take-up gear 150B interacts with or is driven by the second index gear 130B. Since the second index gear 130B rotates in the second opposing direction, the second top sheet take-up gear 150B is driven to rotate in the first direction to wind up the top sheet 166B of the second blister strip 160B.

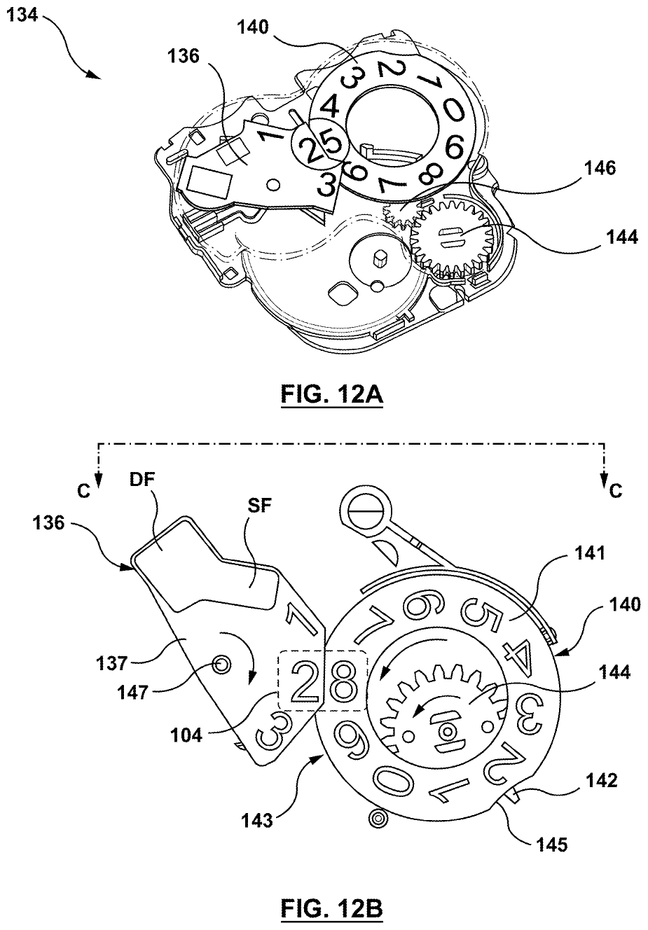

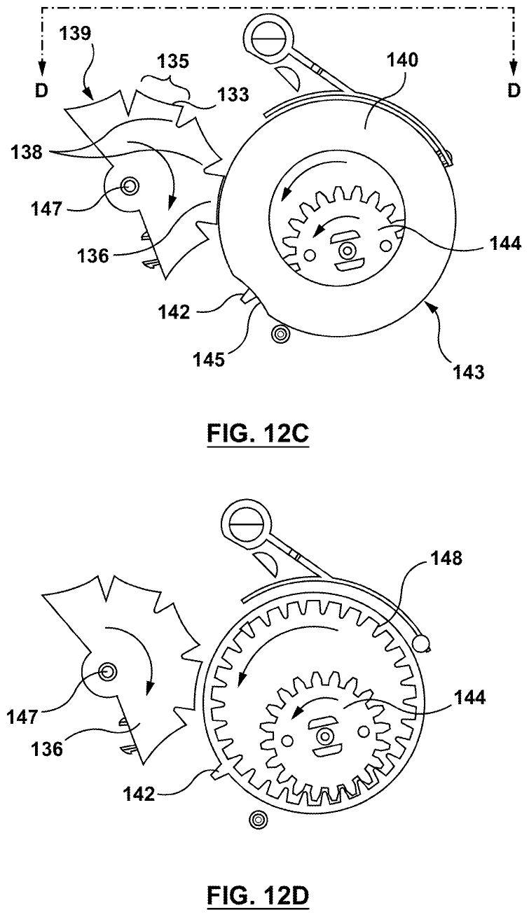

Turning now to FIGS. 12A-12D, the counter subassembly 134 will be described in more detail. The dispensing subassembly 120 advantageously directly drives the counter subassembly 134, so that the dose counter is incremented automatically and simultaneously with a medicament dose being indexed or delivered by the inhaler device 100. As such, beyond operating the mouthpiece cover 108 to actuate the dispensing subassembly 120, the user is not required to perform any additional operating steps to update the dose counter. The dose counter is incremented automatically when the mouthpiece cover 108 is opened, so it is intuitive to the user what the dose counter relates to.

The counter subassembly 134 includes a first count wheel or units ring 140 and a second count component or tens mechanism 136. The first count wheel 140 is driven from the dispensing subassembly 120 of the inhaler device 100 to rotate a fixed angle per dose, and in this embodiment is configured to display a second digit of a two-digit number of the available dose count. The second count component 136 is driven intermittently from the first count wheel 140 such that it rotates a fixed angle per revolution of the first count wheel 140, as will be described in more detail below. In the present embodiment, the second count component 136 displays a first digit of the two-digit number of the available dose count. As such, the first count wheel 140 and the second count component 136 collectively display a number of doses remaining within the inhaler device 100, as shown in FIGS. 12A and 12B. Although the embodiments of the counter subassemblies described herein display a two-digit number, it will be understood by one of ordinary skill in the art that the counting indicia may be revised to display a three-digit number if the total number of doses in the inhaler device exceed one-hundred.

A front or indicia-displaying face or surface 141 of the first count wheel 140 includes counting indicia disposed thereon that includes units or ‘ones’ digits. More particularly, as shown in FIGS. 12A and 12B, the counting indicia of the first count wheel 140 includes the digits 0, 1, 2, 3, 4, 5, 6, 7, 8, 9 in a circular pattern near an outer peripheral edge of the indicia—displaying surface 141. The angle between each digit is the same as the angle of rotation of the first count wheel 140 per dose. The indicia-displaying surface 141 is planar.

A front or indicia-displaying face or surface 137 of the second count component 136 includes counting indicia disposed thereon that includes ‘tens’ digits. The counting indicia of the second count component 136 includes the digits 3, 2, 1 and may also include a single flag SF and a double flag DF near an outer peripheral edge of the indicia-displaying surface 137. The angle between each counting indicia or digit is the angle of rotation of the second count component 136 for every revolution of the first count wheel 140. The indicia-displaying surface 137 is planar.

The display window 104 in the housing 102 is positioned such that one digit on the first count wheel 140 and one digit on the second count component 136 are visible within the display window, and adjacent to each other to form a two-digit number. The displayed digit on the second count component 136 reflects or tracks the number of ‘tens’ of doses remaining in the inhaler device 100, while the displayed digit on the first count wheel 140 reflects or tracks the number of ‘ones’ of doses remaining. When the number of remaining doses is below ten, the second count component 136 will display the single flag SF in the display window 104 instead of or in place of a zero digit. In an embodiment, for example, the single flag SF may be a colored block with no digits thereon to indicate to the user that they are nearing the end of available doses within the inhaler device 100. When the number of remaining doses reaches zero, the second count component 136 will display the double flag DF in the display window 104 instead of a digit. In an embodiment, for example, the double flag DF may be a colored block with no digits thereon that covers the digit of the first count wheel 140 to give clear visual feedback to the user that no doses remain within the inhaler device 100. The double flag DF is configured to cover the digit of the first count wheel 140 when no doses remain within the inhaler device 100.

In the embodiment of FIGS. 12A-12D, the units or ‘ones’ digits are circumferentially disposed on the first count wheel 140 in a descending order in a first direction, and the ‘tens’ digits are disposed near a perimeter of the second count component 136 in a descending order in a second opposing direction. While the counter subassemblies described herein display a number of doses remaining within the inhaler device 100, it will be understood by one of ordinary skill in the art that the counter subassemblies may be modified to display a number of doses delivered by the inhaler device by reversing the order of the counting indicia disposed on the counting subassembly.

Turning now to FIGS. 12C and 12D, the structure and operation of the first count wheel 140 and the second count component 136 will be described in more detail. FIG. 12C is a sectional view taken along line C-C of FIG. 12B, which is along an intermediate position between the indicia-displaying surfaces and the opposing back surfaces of the count subassembly. FIG. 12D is a sectional view taken along line D-D of FIG. 12C, which is adjacent to the opposing back surfaces of the count subassembly. The relative terms “front” and “back” are utilized herein for sake of illustration only and relate to how an inhaler device is customarily positioned by a user during use with a front of the inhaler device including the display window for the counter mechanism.

The first count wheel 140 is an annular or ring-shaped component having the front or indicia-displaying surface 141 and an opposing back surface which includes a counter gear 148, as best shown in the sectional view of FIG. 12D. The counter gear 148 may be integrally formed on or attached to the first count wheel 140. An outer circumferential surface or side 143 extends between the front and back surfaces of the first count wheel 140. The outer circumferential side 143 may be stepped, with a smaller diameter along the back surface of the first count wheel 140 and a larger diameter along the front surface of the first count wheel 140. The stepped nature of the outer circumferential side 143 is apparent via a comparison of the relative diameters thereof in FIGS. 12C and 12D. Stated another way, the indicia-displaying surface 141 of the first count wheel 140 has a greater outer diameter than the counter gear 148.

Adjacent to the counter gear 148, the first count wheel 140 includes a single tooth or protrusion 142 extending radially outward from the outer circumferential side 143. Along the front or indicia-displaying surface 141 of the first count wheel 140, the outer circumferential side 143 is generally circular with a clearance cutout or indent 145 formed around the single tooth 142. The function of the clearance indent 145 will be described in more detail below.

The second count component 136 is configured to rotate around a pivot point 147. The second count component 136 is a non-annular or partial-disk component having the front or indicia-displaying surface 137 and an opposing back surface which includes a plurality of notches 138, as shown in the sectional views of FIGS. 12C and 12D. An outer surface or side 139 extends between the front and back surfaces of the second count component 136. The outer side 139 may be stepped, with a smaller radial dimension along the back surface of the second count component 136 and a larger radial dimension along the front surface of the second count component. The stepped nature of the outer side 139 is apparent via a comparison of the relative diameters thereof in FIGS. 12B and 12C.

The plurality of notches 138 are formed in the outer side 139 of the second count component 136 and do not extend through the front or indicia-displaying surface 137. In an embodiment, the second count component 136 includes four notches 138 but the number of notches is exemplary and depends on the capacity or total number of available doses within the inhaler device 100. Each notch 138 extends radially inward toward the pivot point 147 of the second count component 136 and is configured to mate with or receive the single tooth 142 of the first count wheel 140. The outer side 139 of the second count component 136 may be considered to include a plurality of segments 135, with each segment 135 extending between two neighboring or adjacent notches 138. Along each segment 135, the outer side 139 of the second count component 136 is concave and forms an inverted curved or arc-shaped indent 133 that is coincident with the outer circumferential side 143 of the first count wheel 140.

The first count wheel 140 is driven by the second bottom sheet take-up gear 128B of the dispensing subassembly 120 such that the first count wheel 140 rotates a fixed angle every time a dose is dispensed. More specifically, a transfer gear 144 is attached to an opposing end of the second spindle 129 B of the second bottom sheet take-up gear 128B so that the transfer gear 144, the second spindle 129B, and the second bottom sheet take-up gear 128B rotate simultaneously as an assembly. When the mouthpiece cover 108 is opened, the transfer gear 144 thus rotates in the second opposing direction with the second bottom sheet take-up gear 128B. The transfer gear 144 mates with or directly drives an idler gear 146 to rotate in the first direction, and the idler gear 146 mates with or directly drives the counter gear 148 to rotate in the second opposing direction. Thus, the counter gear 148 rotates in the same direction as the transfer gear 144. As will be apparent to those of ordinary skill in the art, rotation of the transfer gear 144 and the counter gear 148 in the same direction may also be accomplished via a gear train in which the transfer gear 144 directly drives the counter gear 148 in the same direction. For example, as depicted in FIGS. 12B-12D, the desired rotation scheme is accomplished when the counter gear 148 is an internal gear and the transfer gear 144 is a spur or planetary gear so that the transfer gear 144 meshes with or directly drives the internal counter gear 148 in the same direction. The first count wheel 140 is attached to the counter gear 148 to rotate therewith as an assembly, and thus the first count wheel 140 rotates in the second opposing direction when the mouthpiece cover 108 is opened. Thus, various suitable gear train may be utilized herein such that the transfer gear 144 and the counter gear 148 rotate in the same direction.

As best shown on FIG. 12C, the second count component 136 is positioned adjacent or next to the outer circumferential side 143 of the first count wheel 140, such that the single tooth 142 of the first count wheel 140 engages a notch 138 of the plurality of notches 138 of the second count component 136 once per revolution of the first count wheel 140 to intermittently rotate the second count component 136. Stated another way, each time that the first count wheel 140 makes a complete revolution, the single tooth 142 engages a notch 138 of the second count component 136 and turns or rotates the second count component 136 a fixed amount. Thus, there is a fixed rotation of the second count component 136 once per revolution of the first count wheel 140. Since the first count wheel 140 directly drives the second count component 136, the second count component 136 rotates in an opposing direction than the first count wheel 140. In the embodiment of FIGS. 12A-12D, the second count component 136 is configured to rotate in the first direction and the first count wheel 140 is configured to rotate in the second opposing direction. The second count component 136 is stationary and does not rotate when the single tooth 142 of the first count wheel 140 is not engaged a notch 138 of the second count component 136.

The second count component 136 is disposed next to or side-by-side in a common plane with the first count wheel 140, and thus the second count component 136 rotates on a different axis of rotation than the first count wheel 140. Stated another way, the first count wheel 140 rotates about a first axis and the second count component 136 rotates about a second axis, with the second axis being parallel to and spaced apart from the first axis.

As stated above, along each segment 135, the outer side 139 of the second count component 136 is concave and forms the arc-shaped indent 133 that is coincident with the outer circumferential side 143 of the first count wheel 140. As best shown on FIG. 12C, the geometry of the arc-shaped indents 133 is coincident with the circular profile of the first count wheel 140 to prevent the second count component 136 from inadvertently rotating when not engaged with the first count wheel 140. Particular, this geometry or profile of the outer side 139 of the second count component 136 ensures that the second count component 136 does not rotate, and remains stationary, when the single tooth 142 of the first count wheel 140 is not engaged with one of the notches 138 of the second count component 136. Conversely, when the single tooth 142 of the first count wheel 140 is engaged with or received within one of the notches 138 of the second count component 136, the clearance indent 145 of the first count wheel 140 allows the second count component 136 to briefly rotate with the first count wheel 140 to change the tens display.

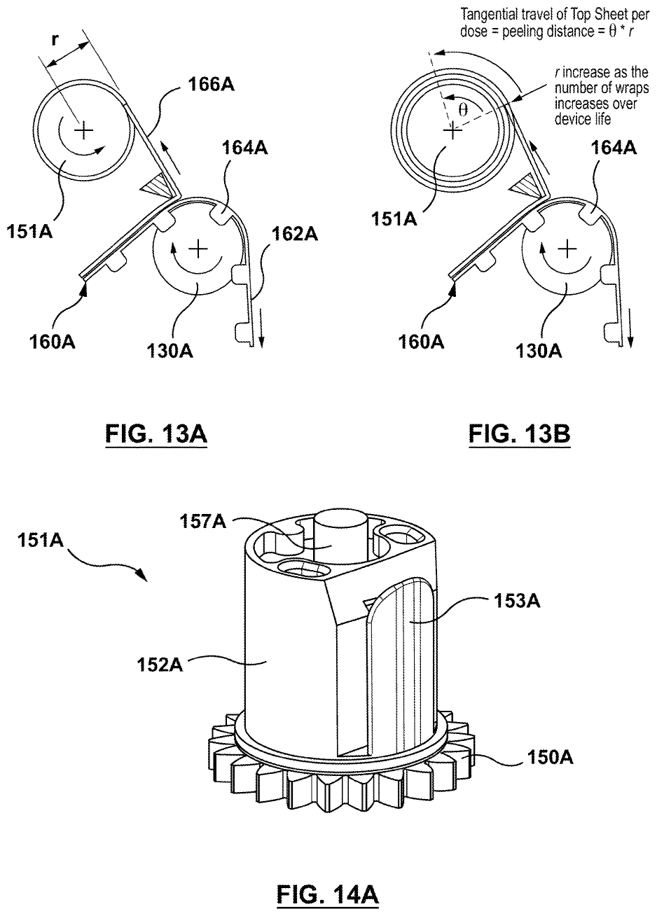

Turning now to FIGS. 13A, 13B and 14A-14E, first and second tensioning mechanisms 151A, 151B will be described in more detail. The tensioning mechanisms 151A, 151B function to peel the top sheets 166A, 166B from the first and second blister strips 160A, 160B, respectively, in a manner that maintains consistent peeling distance or amount over the device lifetime. More particularly, the tensioning mechanisms 151A, 151B ensure that the peeling distances of the top sheets 166A, 166B are configured to properly open the pockets 164A, 164B for each dose in order to achieve effective dispensing of the powdered medicaments 168A, 168B to the user. A pocket may not be fully exposed if the peeling distance or amount is too low, which makes it more difficult to achieve sufficient evacuation of the powdered medicament disposed therein upon inhalation. Further, if the peeling distance or amount is too high, the next or following pocket may be prematurely exposed and there is a risk of some medicament disposed therein being lost, which could result in an under dose on the next dispense. The tensioning mechanisms 151A, 151B also function to maintain sheet tension of the top sheets 166A, 166B over the device lifetime. The top sheets 166A, 166B need to be under consistent tension to ensure proper operation of the inhaler device 100. The tension of each top sheet 166A, 166B is linked to the force required by the user to operate the inhaler device 100 and move the mouthpiece cover 108. Ensuring consistent tension in the top sheets 166A, 166B thus provides a more consistent user experience over the device lifetime. Maintaining consistent tension in the top sheets 166A, 166B also results in more consistent and lower peak mechanical stress in the top sheets 166A, 166B and the surrounding components, which reduces the risk of mechanical failure during use.

The first tensioning mechanism 151A is associated with the first top sheet take-up gear 150A, for winding up the top sheet 166A of the first blister strip 160A, and the second tensioning mechanism 151B is associated with a second top sheet take-up gear 150B (see FIGS. 9A and 9B), for winding up the top sheet 166B of the second blister strip 160B. Only first tensioning mechanism 151A is described herein for sake of brevity, as the second tensioning mechanism 151B operates in the same manner. It will be apparent to those of ordinary skill in the art that certain features or components of the second tensioning mechanism 151B (i.e., the cam surface described herein) will be modified to extend in an opposing direction from the description below such that the second tensioning mechanism 151B is configured to operate in an opposite direction than the first tensioning mechanism 151A. Advantageously, the first tensioning mechanism 151A and the second tensioning mechanism 151B use some components of identical design (i.e., the compression spring and the take-up hub described herein), which may reduce the cost of manufacturing and assembly compared to other inhaler devices in which all components need to be manufactured individually for each side of the device in order to operate in opposite directions.

The tensioning mechanism 151A includes the first top sheet take-up gear 150A, the take-up hub 152A having a hook 153A integrally formed thereon or fixed thereto, a base 154A having a cam surface 155A integrally formed thereon or fixed thereto, a nut 156A, a shaft 157A, and a compression spring 158A that extends or is disposed between the nut 156A and a top end of the take-up hub 152A. The compression spring 158A biases the nut 156A downwards, towards the base 154A, into the cam surface 155A. The compression spring 158A is disposed about or around the shaft 157A and is longitudinally or axially adjacent to the nut 156A, and the take-up hub 152A is disposed about or around the nut 156A and the compression spring 158A. Stated another way, the take-up hub 152A encircles or surrounds the nut 156A and the compression spring 158A which are contained therein.

The top sheet 166A of the first blister strip 160A is secured or attached to the take-up hub 152A via the hook 153A such that as the take-up hub 152A rotates, the top sheet 166A of the first blister strip 160A wraps around it. The hook 153A is configured to attach to a leading end of the top sheet 166A such that rotation of the take-up hub 152A results in winding or wrapping of the top sheet 166A around the take-up hub 152A. Since the take-up hub 152A completes multiple rotations over the lifetime of the inhaler device 100, the top sheet 166A of the first blister strip 160A wraps over itself multiple times, causing its radial position on the take-up hub 152A to increase, i.e., a radial distance of each subsequent winding of the top sheet 166A from the take-up hub 152A increases with each winding. Since rotation of the base 154A corresponds to the rotation of the first top sheet take-up gear 150A, the base 154A rotates a fixed amount for each dose and the distance that the top sheet 166A of the first blister strip 160A is peeled relative to the bottom sheet 162A is determined by the tangential travel of the top sheet 166A of the first blister strip 160A at the take-up hub 152A. This tangential travel may be calculated as θ* r, where θ is the angle of rotation of the take-up hub 152A per dose, and r is the radial position of the top sheet 166A of the first blister strip 160A on the take-up hub 152A. As the radial position of the top sheet 166A of the first blister strip 160A increases, this tangential distance therefore increases for a fixed rotation of the take-up hub 152A and, without any modification of the mechanism, the top sheet 166A of the first blister strip 160A would be peeled further relative to the bottom sheet 162A later in the device life. This is known as the wrapping effect and is illustrated via a comparison of FIG. 13A and FIG. 13B. FIG. 13A is a schematic view of the first blister strip 160A early in the device life, and FIG. 13B is a schematic view of the first blister strip 160A late in the device life. FIG. 13B illustrates how the effective diameter of the take-up hub 152A increases as the first blister strip 160A is wrapped around the take-up hub 152A.

In order to ensure that the top sheet 166A of the first blister strip 160A is peeled the same amount each dose and compensate for the wrapping effect, the tensioning mechanism 151A includes the cam surface 155A, the nut 156A, and the compression spring 158A. The function of the cam surface 155A, the nut 156A, and the compression spring 158A is to provide a constant driving tension to the top sheet 166A over the entire strip length. As will be described in more detail herein, increased tension along the top sheet 166A results in rotation of the take-up hub 152A relative to the base 154A to reduce the tension along the top sheet 166A. When the take-up hub 152A rotates relative to the base, the compression spring 158A is compressed and axial compression of the compression spring 158A is transferred to torque applied to the take-up hub 152A.

With reference to FIGS. 14B-14D, the structure of the tensioning mechanism 151A will be described in more detail. The base 154A is attached to or formed integrally with the first top sheet take-up gear 150A to rotate as an assembly when the first top sheet take-up gear 150A is rotationally driven. For example, the base 154A may include a plurality of gear teeth integrally formed with or fixed to an outer circumferential surface thereof to form the first top sheet take-up gear 150A. The shaft 157A extends from the base 154A and is attached to or formed integrally with the base 154A to rotate therewith. Thus, the shaft 157A, the base 154A, and the first top sheet take-up gear 150A rotate as an assembly when the first top sheet take-up gear 150A is rotationally driven.

The nut 156A is disposed between and coupled to each of the take-up hub 152A and the base 154A. The nut 156A is disposed about or around the shaft 157A and is coupled to the base 154A via at least one inwardly-extending rib 119A that projects or extends radially inwards from an inner circumferential surface of the nut 156A. In an embodiment, the nut 156A includes a plurality of inwardly-extending ribs 119A that act as a cam follower. In an embodiment, the plurality of inwardly-extending ribs 119A are circumferentially spaced apart in equal increments. More particularly, as best shown on FIG. 14C, the inwardly-extending ribs 119A of the nut 156A are disposed onto and engage the cam surface 155A of the base 154A. Further, the nut 156A is coupled to the take-up hub 152A via a splined coupling 159A such that the take-up hub 152A rotates with the nut 156A and no relative rotation is permitted therebetween. Stated another way, due to the splined coupling 159A, the take-up hub 152A is rotationally locked to the nut 156A such that the nut 156A and the take-up hub 152A rotate as an assembly. As best shown on FIG. 14C, the splined coupling 159A includes an outwardly-extending rib 117A which projects or extends radially outwards from an outer circumferential surface of the nut 156A and is received within an axial slot 115A of the take-up hub 152A. The outwardly-extending rib 117A is permitted to slide or move in an axial direction along the axial slot 115A such that the nut 156A is permitted to slide or move in an axial direction relative to the take-up hub 152A, but the outwardly-extending rib 117A does not permit the nut 156A to rotate relative to the take-up hub 152A.

When the base 154A rotates in the second opposing direction with the first top sheet take-up gear 150A, the nut 156A and the take-up hub 152A also rotate in the second opposing direction due to the interaction between the nut 156A with the compression spring 158A and the cam surface 155A of the base 154A. More particularly, as the base 154A is being rotationally driven in the second opposing direction, the take-up hub 152A is configured to rotate in the second opposing direction via engagement of the inwardly-extending ribs 119A of the nut 156A with the cam surface 155A. As shown on FIG. 14D, the cam surface 155A includes alternating sections of vertical surfaces 111A and angled or inclined surfaces 113A. Vertical surfaces 111A extend generally parallel to a longitudinal axis of the shaft 157A. As the base 154A is being rotationally driven in the second opposing direction (i.e., the counter-clockwise direction in this embodiment), the nut 156A and the take-up hub 152A rotationally locked therewith rotate with the base 154A in the second opposing direction. Interaction between the compression spring 158A, the nut 156A and the cam surface 155A result in a torque being applied from the cam surface 155A to the nut 156A, in the direction that would push the nut 156A down the cam surface 155A. The compression spring 158A is pushing the nut 156A against the inclined surfaces 113A of the cam surface 155A, which results in torque acting on the take-up hub 152A that drives the take-up hub 152A in the second opposing direction. As a result of this interaction when the base 154A moves counter-clockwise, the nut 156A and the take-up hub 152A also rotate counter-clockwise with the base 154A. It will be understood by one of ordinary skill in the art that the pattern of the cam surface 155A is exemplary. A thread or other ramped surface may be utilized as the cam surface 155A.

Although the take-up hub 152A rotates in the second opposing direction with the base 154A due to the nut 156A interacting with the compression spring 158A and the cam surface 155A of the base 154A, relative rotation between the take-up hub 152A and the base 154A is permitted. More particularly, when sufficient torque is applied between the take-up hub 152A and the base 154A, the take-up hub 152A will rotate relative to the base 154A. Since the take-up hub 152A is permitted to rotate relative to the base 154A in a direction that will reduce tension in the top sheet 166A (i.e., in the first direction), this relative rotation stabilizes or balances the tension in the top sheet 166A via deflection of the compression spring 158A. The tensioning mechanism 151A is therefore acting as a torsion or torque limiter between the take-up hub 152A and the base 154A to control the tension in the top sheet 166A of the first blister strip 160A.

In addition to the torque being applied from the cam surface 155A to the nut 156A, there is also a torque in the opposing direction acting on the nut 156A from its interaction with the take-up hub 152A via the outwardly-extending rib 117A. This opposing torque comes from the tension in the top sheet 166A, which is acting to apply torque to the take-up hub 152A. The two opposing torques on the nut 156A are in equilibrium, and as such the compression spring 158A (via the nut 156A and cam surface 155A) effectively balances the tension in the top sheet 166A. If the tension in the top sheet 166A increases, then the nut 156A will move further up the cam surface 155A and the spring force of the compression spring 158A will increase to compensate for the increased tension.

More particularly, due to the wrapping effect described above, as the take-up hub 152A rotates, the radial position of the top sheet 166A of the first blister strip 160A increases. As a result of this increased radial position, the take-up hub 152A attempts to peel a longer length of top sheet 166A and the tension on the top sheet 166A increases due to a change in the peeling angle between the top sheet 166A of the first blister strip 160A and the bottom sheet 162A of the first blister strip 160A. When tension on the top sheet 166A increases, the torque that the top sheet 166A applies to the take-up hub 152A increases as well. Rather than the torque continuing to increase, the take-up hub 152A will start to rotate relative to the base 154A in the second direction, and the nut 156A will move helically up the cam surface 155A of the base 154A, thereby compressing the compression spring 158A. More particularly, when the take-up hub 152A and the nut 156A rotationally locked thereto begin to rotate in the second direction due to the increased tension on the top sheet 166A, the base 154A and the cam surface 155A remain stationary and the inwardly-extending ribs 119A of the nut 156A move along the inclined surfaces 113A of the cam surface 155A in a direction towards the compression spring 158A, i.e., higher up the cam surface 155A. The compression spring 158A compresses as the nut 156A presses against it. As the nut 156A moves relative to the base 154A, the nut 156A also moves axially relative to the take-up hub 152A because the outwardly-extending rib 117A of the nut 156A is permitted to move axially within the axial slot 115A of the take-up hub 152A. Thus, rotation of the take-up hub 152A relative to the base 154A results in axial movement of the nut 156A relative to the take-up hub 152A and relative to the base 154A, and further axial movement of the nut 156A towards the compression spring 158A axially compresses the compression spring 158A. Since the take-up hub 152A is rotationally locked to the nut 156A via the splined coupling 159A, the nut 156A transfers the axial force of the compressed spring 158A into torque on the take-up hub 152A. The combination of the compression spring 158A, the nut 156A, and the cam surface 155A is thereby providing torque to react or counteract the relative rotation between the take-up hub 152A and the base 154A, which is acting to maintain consistent tension in the top sheet 166A.

The take-up hub 152A is axially constrained relative to the base 154A via a clip or retention feature 149A disposed between the shaft 157A and the take-up hub 152A. More particularly, since the compression spring 158A is acting to axially separate the take-up hub 152A and the base 154A, the retention feature 149A (best shown on FIG. 14B) is disposed between the top end of the take-up hub 152A and the compression spring 158A to maintain the correct relative axial position between the take-up hub 152A and the base 154A. The retention feature 149A may be a clip, a bayonet, or other component suitable to maintain the correct relative axial position between the take-up hub 152A and the base 154A. In another embodiment (not shown), the retention feature may be attached to the interior of the housing 102 to maintain the correct relative axial position between the take-up hub 152A and the base 154A.

FIG. 14E illustrates how the position of the nut 156A changes over the lifetime of the device. In its initial assembled state shown in the left image, prior to attachment of the take-up hub 152A to the first blister strip 160A, the nut 156A rests at the bottom end of the cam surface 155A. The cam surface 155A is designed such that, in this position, there is no resultant torque between the take-up hub 152A and the base 154A, even though there may be axial force from the compression spring 158A. When the device is assembled, the top sheet 166A is assembled under some tension to ensure that peeling is effective from the first dose and as a result, the nut 156A is lifted up the cam surface 155A slightly away from the vertical surfaces 111A of the cam surface 155A. More particularly, as shown in the middle image, when the device is assembled the top sheet 166A is attached to the take-up hub 152A, the take-up hub 152A is rotated relative to the base 154A, and the nut 156A moves up the cam surface 155A. The compression spring 158A is deflected or slightly compressed from its uncompressed length so that the compression spring 158A has a preload force. The preload force ensures that the tension in the top sheet 166A of the first blister strip 160A is sufficiently high to peel it away from the bottom sheet 162A of the first blister strip 160A at the start of the device life. The specifications of the compression spring 158A and the angle of the cam surface 155A should be configured to provide a minimum tension in the top sheet 166A of the first blister strip 160A that is higher than the maximum force required to peel the top sheet 166A of the first blister strip 160A from the bottom sheet 162A of the first blister strip 160A. In this assembled state, the combination of the compression spring 158A, the nut 156A, and the cam surface 155A provide a torque between the base 154A and the take-up hub 152A, which is reacted by tension in the top sheet 166A. Over the lifetime of the inhaler device 100 as shown in the right image, the tension in the top sheet 166A of the first blister strip 160A increases. Due to the increased tension of the top sheet 166A, the nut 156A moves further up the cam surface 155A so that the increased tension of the top sheet 166A is balanced by further deflection of the compression spring 158A.

The angle or slope of each inclined surface 113A of the cam surface 155A is configured to maintain consistent sheet tension of the top sheets 166A, 166B over the device lifetime. As described above, the top sheets 166A, 166B need to be under consistent tension to ensure proper operation of the inhaler device 100. In general, the angle or slope of each inclined surface 113A of the cam surface 155A is selected to ensure that the nut 156A moves along the cam surface 155A of the base 154A during operation of the inhaler device 100 and does not move beyond or past the vertical surfaces 111A. In an embodiment, the inclined surfaces 113A of the cam surface 155A extend at an angle between 35 degrees and 55 degrees relative to the longitudinal axis of the base 154A. In an embodiment, the inclined surfaces 113A of the cam surface 155A extend at an angle between 40 degrees and 50 degrees relative to the longitudinal axis of the base 154A. In an embodiment, the inclined surfaces 113A of the cam surface 155A extend at an angle of approximately 45 degrees relative to the longitudinal axis of the base 154A, with approximately as used herein including a tolerance of three degrees. In an embodiment, the inclined surfaces 113A of the cam surface 155A have a slope between 0.70 and 1.0. In an embodiment, the inclined surfaces 113A of the cam surface 155A have a slope between 0.80 and 0.95. In another embodiment, the inclined surfaces 113A of the cam surface 155A have a slope between 0.7 and 1.4. In another embodiment, the inclined surfaces 113A of the cam surface 155A have a slope between 1.0 and 1.4. The slope of the inclined surfaces 113A of the cam surface 155A may be constant over the length of the inclined surface, or may vary over the length of the inclined surface.

The radial width of the inclined surface 113A of the cam surface 155A is configured to optimize the amount of friction between the nut 156A and the cam surface 155A. In general, larger dimensions of the radial width of the inclined surface 113A of the cam surface 155A result in increased friction between the components while smaller dimensions of the radial width of the inclined surface 113A of the cam surface 155A may result in the nut 156A undesirably falling off the cam surface 155A. In an embodiment, each inclined surface 113A of the cam surface 155A has a radial width between 1 mm and 3 mm. In an embodiment, each inclined surface 113A of the cam surface 155A has a radial width between 1.5 mm and 2.5 mm. In an embodiment, each inclined surface 113A of the cam surface 155A has a radial width of approximately 2 mm, with approximately as used herein including a tolerance of 0.2 mm.

Compared to tensioning mechanisms that utilize a torsion spring to maintain consistent sheet tension, the use of the compression spring 158A within the tensioning mechanism 151A may reduce cost and environmental impact of the inhaler device 100 and also may result in a simpler assembly and/or manufacturing of the inhaler device 100. Particularly, for instance, the compression spring 158A does not require hooks or legs that need to be formed on the ends of a torsion spring. Since there are no legs to engage with retention features, the compression spring 158A may be considerably easier to manufacture and/or assemble than a torsion spring. Further, the compression spring 158A is believed to create a more axis-symmetric and balanced torque on the take-up hub 152A as compared to a mechanism that utilizes a torsion spring so that a take-up hub according to embodiments hereof does not tend to be rocked off axis, which could cause a top sheet of a blister strip to travel axially and tangle or drag as it wraps around the take-up hub.

It will be understood by one of ordinary skill in the art that the relative dimensions and/or arrangement of the various components of the tensioning mechanism 151A may vary from that shown in the embodiment of FIGS. 14A-14E. Variation in the relative dimensions and/or arrangement of the various components may result in different mechanical properties of the mechanism due to differences in properties such as a contact radii, a slope of the cam surface, and the compression spring specification. Variation in the relative dimensions and/or arrangement of the various components provides alternative options which may be desired in some applications, depending on factors such as space requirements, manufacturing or assembly methods, and the like.

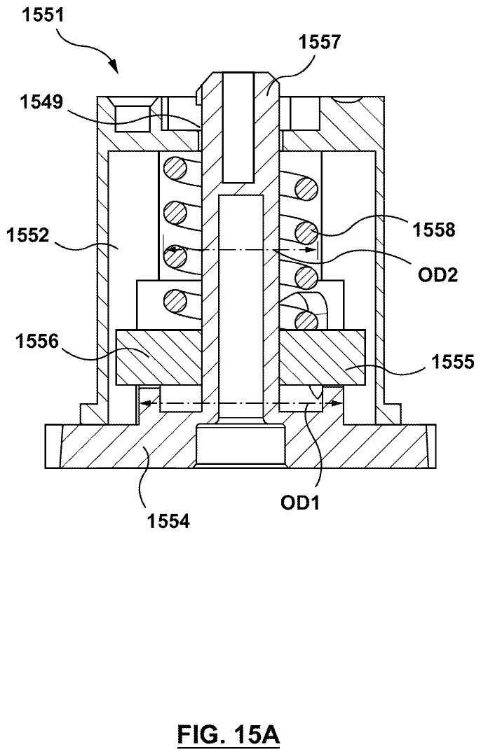

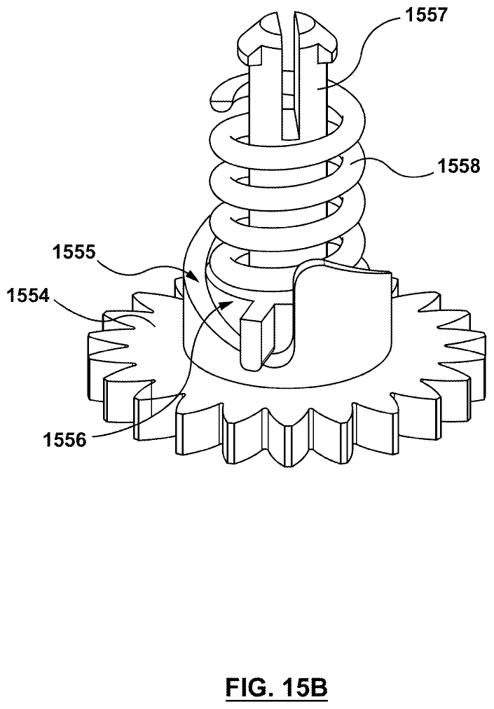

For example, FIGS. 15A and 15B illustrate another embodiment of a tensioning mechanism 1551 having different relative dimensions than the tensioning mechanism 151A. The tensioning mechanism 1551 includes a hub 1552, a base 1554 having a cam surface 1555 integrally formed thereon or fixed thereto, a nut 1556, a shaft 1557, and a compression spring 1558 that extends or is disposed between the nut 1556 and an end of the take-up hub 1552 that is spaced apart from the base 1554. The take-up hub 1552 is axially constrained relative to the base 1554 via a clip or retention feature 1549 disposed between the shaft 1557 and the take-up hub 1552. The tensioning mechanism 1551 operates the same as the tensioning mechanism 151A described above, but in this embodiment the cam surface 1555 on the base 1554 has a larger diameter than a diameter of the compression spring 1558. Stated another way, the cam surface 1555 has a first outer diameter OD1 and the compression spring 1558 has a second outer diameter OD2, the first outer diameter being greater than the second outer diameter. This arrangement may result in different mechanical properties of the tensioning mechanism 1551 due to differences in the contact radii, the slope of the cam surface, and the compression spring specification.

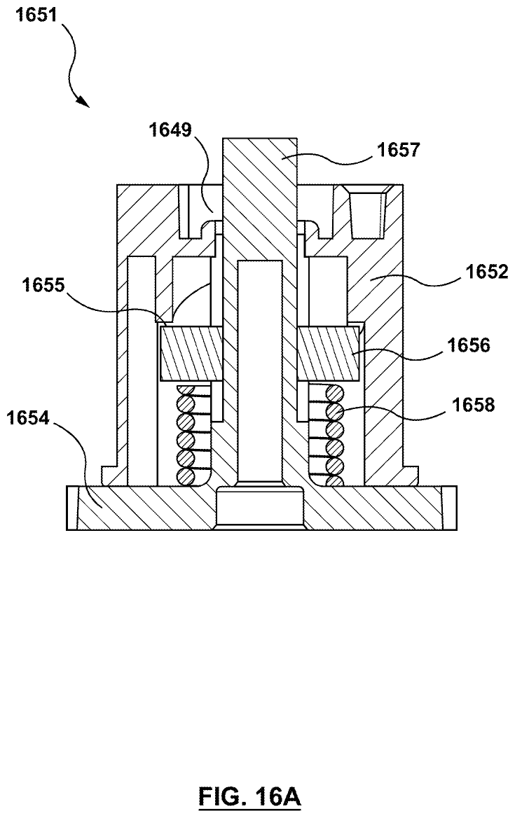

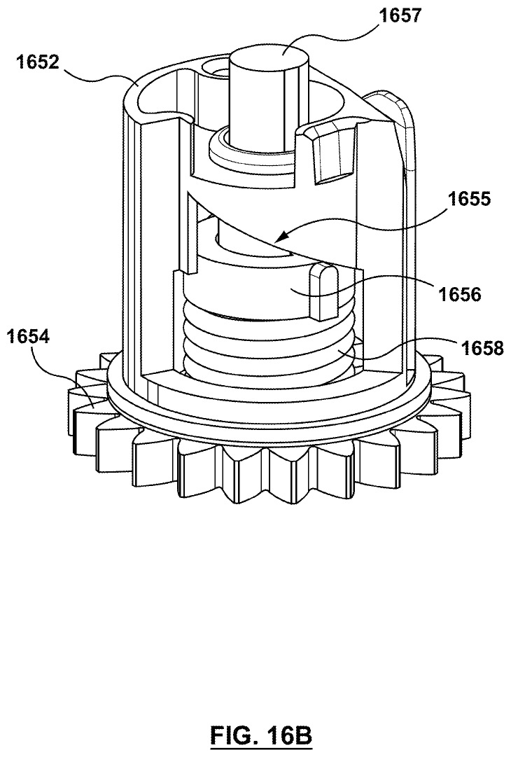

As another example, FIGS. 16A and 16B illustrate another embodiment of a tensioning mechanism 1651 in which the components thereof are arranged in a different manner than the components of the tensioning mechanism 151A. The tensioning mechanism 1651 includes a hub 1652, a base 1654, a nut 1656, a shaft 1657, and a compression spring 1658. The take-up hub 1652 is axially constrained relative to the base 1654 via a clip or retention feature 1649 disposed between the shaft 1657 and the take-up hub 1652. The tensioning mechanism 1651 provides a function similar to the tensioning mechanism 151A described above, but in this embodiment a cam surface 1655 is integrally formed on or fixed to an inner circumferential surface of the take-up hub 1652. In order to interact with the cam surface 1655, the nut 1656 includes a plurality of outwardly-extending ribs 1619 that project or extend radially outwards from an outer circumferential surface of the nut 1656. The compression spring 1658 extends or is disposed between the nut 1656 and the base 1654, biasing the nut 1656 upwards into the cam surface 1655. In addition, in this embodiment, the nut 1656 is rotationally locked to the base 1654 and the nut 1656 is permitted to only move axially relative to the base 1654. Stated another way, the nut 1656 is not permitted to rotate relative to the base 1654. The nut 1656 is rotationally locked to the base 1654 via a spline coupling (not shown) that includes an inwardly-extending rib which projects or extends radially outwards from an inner circumferential surface of the nut 1656 and is received within an axial slot of the shaft 1657, as similarly shown the configuration of the nut 156A in FIG. 14D. The inwardly-extending rib is permitted to slide or move in an axial direction along the axial slot such that the nut 1656 is permitted to slide or move in an axial direction relative to the shaft 1657 and base 1654 attached thereto, but the inwardly-extending rib does not permit the nut 1656 to rotate relative to the shaft 1657 and base 1654.

The cam surface 1655, the nut 1656, and the compression spring 1658 interact in a manner that achieves the same function as the cam surface 155A, the nut 156A, and the compression spring 158A and thereby provide a constant driving tension to the first blister strip 160A over the entire strip length. When the tension along the top sheet 166A increases, the take-up hub 1652 rotates relative to the base 1654 to reduce tension along the top sheet 166A.

When the base 1654 rotates with a top sheet take-up gear, such as a first top sheet take-up gear 150A, the nut 1656 and the take-up hub 1652 also rotate in the same direction with the base 1654 due to the interaction between the nut 1656 with the compression spring 1658 and the cam surface 1655 of the take-up hub 1652. Further, relative rotation between the take-up hub 1652 and the base 1654 is permitted. More particularly, when sufficient torque is applied between the take-up hub 1652 and the base 1654, the take-up hub 1652 will rotate relative to the base 1654. Since the take-up hub 1652 is permitted to rotate relative to the base 1654 in a direction that will reduce tension in a top sheet, such as a top sheet 166A, this relative rotation stabilizes or balances the tension in the top sheet via deflection of the compression spring 1658. The tensioning mechanism 1651 is therefore acting as a torsion or torque limiter between the take-up hub 1652 and the base 1654 to control the tension in a top sheet of a blister strip, such as a first blister strip 160A.

When tension on a top sheet of a blister strip increases, the torque that the top sheet applies to the take-up hub 1652 increases as well. Rather than the torque continuing to increase, the take-up hub 1652 will start to rotate relative to the base 1654 and the nut 1656 will move helically down the cam surface 1655 of the take-up hub 1652, thereby compressing the compression spring 1658. More particularly, when the take-up hub 1652 begins to rotate due to the increased tension on the top sheet of the blister strip, the outwardly-extending ribs 1619 of the nut 1656 move along the inclined surfaces of the cam surface 1655 towards the compression spring 1658. The compression spring 1658 compresses as the nut 1656 presses against it. The nut 1656 moves axially relative to the base 1654 because the inwardly-extending rib of the nut 1656 is permitted to move axially within the axial slot of the shaft 1657. Thus, rotation of the take-up hub 1652 relative to the base 1654 results in downward axial movement of the nut 1656 relative to an upper surface of the take-up hub 1652, and toward to the base 1654, and further axial movement of the nut 1656 axially compresses the compression spring 1658. The nut 1656 transfers the axial force of the compression spring 1658 into torque on the take-up hub 1652. The combination of the compression spring 1658, the nut 1656, and the cam surface 1655 is thereby providing torque to counteract the relative rotation between the take-up hub 1652 and the base 1654, which is acting to maintain consistent tension in a top sheet of a blister strip.

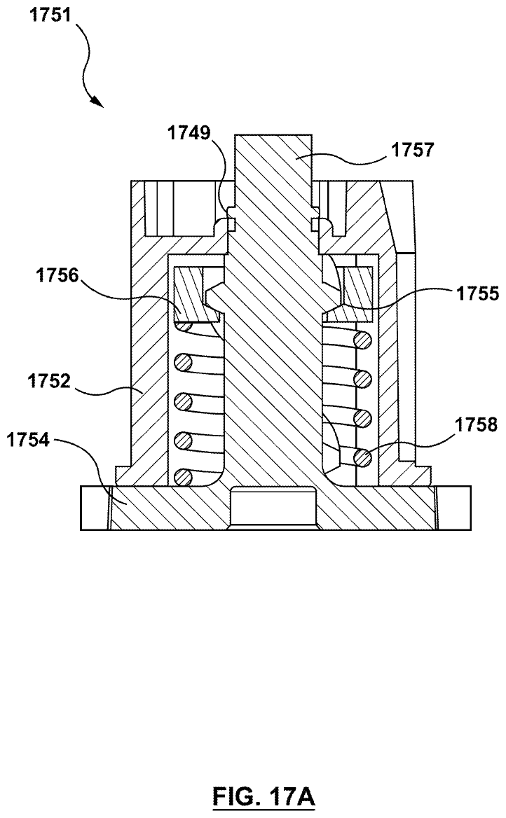

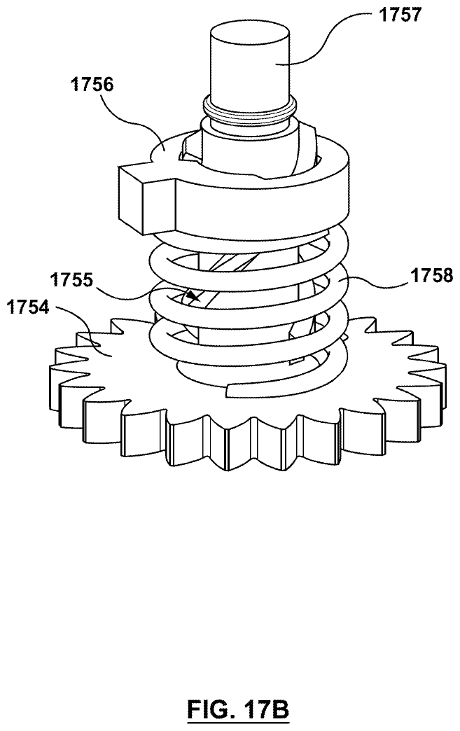

As another example, FIGS. 17A and 17B illustrate another embodiment of a tensioning mechanism 1751 in which the components thereof are arranged in a different manner than the components of the tensioning mechanism 151A. The tensioning mechanism 1751 includes a hub 1752, a base 1754 having a cam surface 1755 integrally formed thereon or fixed thereto, a nut 1756, a shaft 1757, and a compression spring 1758. The take-up hub 1752 is axially constrained relative to the base 1754 via a clip or retention feature 1749 disposed between the shaft 1757 and the take-up hub 1752. The tensioning mechanism 1751 provide the same function as the tensioning mechanism 151A described above, but in a different way. In this embodiment, the compression spring 1758 extends or is disposed between the nut 1756 and the base 1754, biasing the nut 1756 upwards into the cam surface 1755.

The cam surface 1755, the nut 1756, and the compression spring 1758 function the same as the cam surface 155A, the nut 156A, and the compression spring 158A to provide a constant driving tension to a blister strip, such as a first blister strip 160A, over the entire strip length. Increased tension along a top sheet of the blister strip, such as a top sheet 166A, results in rotation of the take-up hub 1752 relative to the base 1754 to reduce the tension along the top sheet of the blister strip.

When the base 1754 rotates with a top sheet take-up gear, such as a first top sheet take-up gear 150A, the nut 1756 and the take-up hub 1752 also rotate in the same direction with the base 1754 due to the interaction between the nut 1756 with the compression spring 1758 and the cam surface 1755. Further, relative rotation between the take-up hub 1752 and the base 1754 is permitted. More particularly, when sufficient torque is applied between the take-up hub 1752 and the base 1754, the take-up hub 1752 will rotate relative to the base 1754. Since the take-up hub 1752 is permitted to rotate relative to the base 1754 in a direction that will reduce tension in a top sheet, such as a top sheet 166A, this relative rotation stabilizes or balances the tension in the top sheet via deflection of the compression spring 1758. The tensioning mechanism 1751 is therefore acting as a torsion or torque limiter between the take-up hub 1752 and the base 1754 to control the tension in a top sheet of a blister strip, such as a first blister strip 160A.

When tension on a top sheet of a blister strip increases, the torque that the top sheet applies to the take-up hub 1752 increases as well. Rather than the torque continuing to increase, the take-up hub 1752 will start to rotate relative to the base 1754 and the nut 1756 will move helically down the cam surface 1755, thereby compressing the compression spring 1758. More particularly, when the take-up hub 1752 begins to rotate due to the increased tension on the top sheet of the blister strip, the inwardly-extending ribs of the nut 1756 move downwardly along the inclined surfaces of the cam surface 1755 towards the compression spring 1758. The compression spring 1758 compresses as the nut 1756 presses against it. The nut 1756 moves axially relative to the base 1754 because an outwardly-extending rib of the nut 1756 is permitted to move axially within an axial slot of the take-up hub 1752 (similar to splined coupling arrangement between the nut 156A and the take-up hub 152A). Thus, rotation of the take-up hub 1752 relative to the base 1754 results in downward axial movement of the nut 1756 relative to a top surface or wall of the take-up hub 1752, and toward the base 1754, and further axial movement of the nut 1756 towards the compression spring 1758 axially compresses the compression spring 1758. The nut 1756 transfers the axial force of the compression spring 1758 into torque on the take-up hub 1752. The combination of the compression spring 1758, the nut 1756, and the cam surface 1755 is thereby providing torque to counteract the relative rotation between the take-up hub 1752 and the base 1754, which is acting to maintain consistent tension in a top sheet of a blister strip.

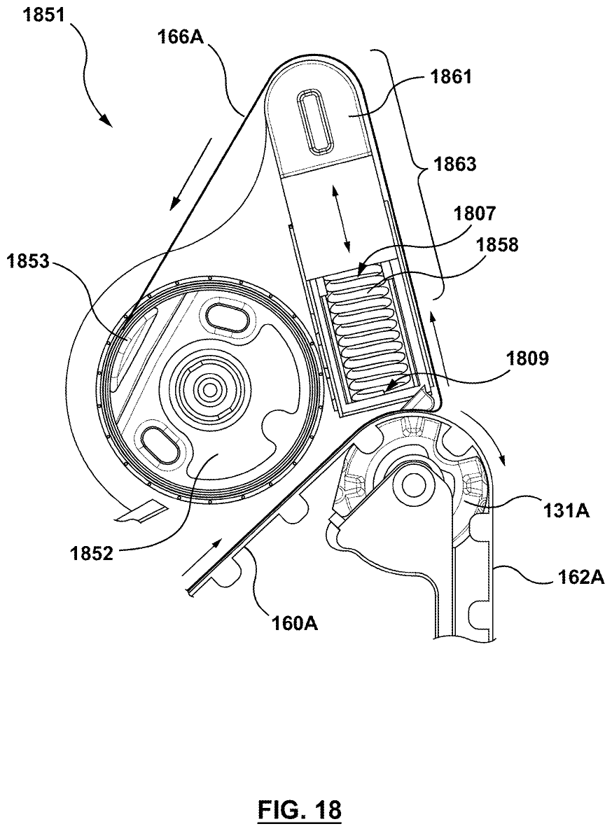

FIGS. 18-20 illustrate another embodiment of a tensioning mechanism or subassembly that may be utilized within the inhaler device 100. Similar to tensioning mechanism 151A described above, a tensioning mechanism 1851 functions to peel a top sheet 166A from a blister strip 160A in a manner that maintains consistent peeling distance or amount over the device lifetime. The tensioning mechanism 1851 also functions to maintain sheet tension of the top sheet 166A over the device lifetime. The tensioning mechanism 1851 is associated with a top sheet take-up gear, such as the first top sheet take-up gear 150A, for winding up the top sheet 166A of the blister strip 160A. It should be understood that in the inhaler device 100 each of the first and second tensioning mechanisms 151A, 151B may be replaced by a tensioning mechanism 1851 without departing from the scope of the present disclosure.

In this embodiment, a top sheet take-up gear is integrally formed with or secured to a take-up hub 1852 to rotate as an assembly. The top sheet 166A of the blister strip 160A is secured or attached to the take-up hub 1852 via a hook 1853 such that when the first take-up hub 1852 rotates, the top sheet 166A of the blister strip 160A wraps around it. As described above, as the take-up hub 1852 completes multiple rotations over the lifetime of the inhaler device 100, the top sheet 166A of the blister strip 160A wraps over itself multiple times, causing its radial position on the take-up hub 1852 to increase.

The tensioning mechanism 1851 includes a slider 1861 and at least one compression spring 1858 attached to the slider 1861. The tensioning mechanism 1851 is disposed between the take-up hub 1852 and an index spool, such as the first index spool 131A, and is configured to adjust the length of the top sheet 166A between the take-up hub 1852 and the first index spool 131A to maintain consistent tension on the top sheet 166A and thereby to compensate for the wrapping effect and component tolerances. As will be described in more detail herein, the tensioning mechanism 1851 applies a non-parallel force to a portion of the top sheet 166A via the compression spring 1858. The tensioning mechanism 1851 is coupled to the housing 102 of the inhaler device 100 so as to permit axial movement of the slider 1861 relative to the housing 102 along a predetermined path 1863. The slider 1861 is configured to receive and guide an intermediate portion of the top sheet 166A, the intermediate portion being disposed between the first index spool 131A and the take-up hub 1852. Increased tension along the top sheet 166A results in axial movement of the slider along the predetermined path 1863, and the axial movement of the slider 1861 axially compresses the compression spring 1858 to reduce the tension along the top sheet 166A.

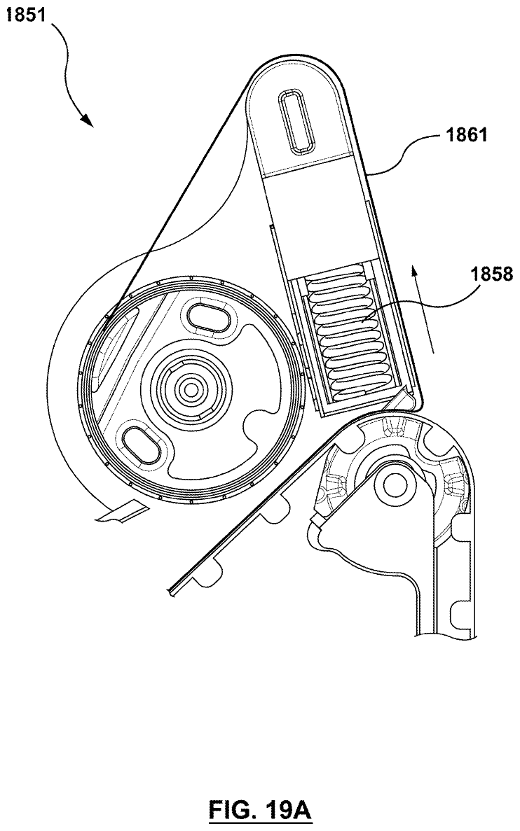

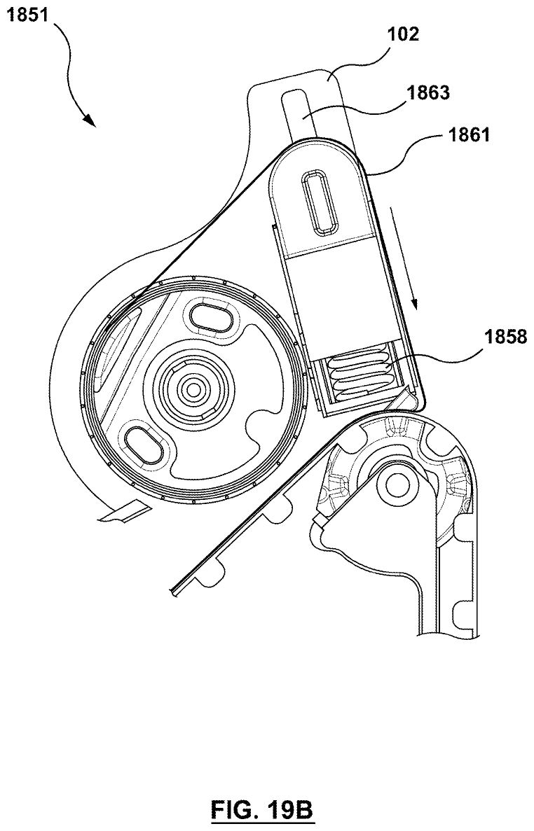

More particularly, the slider 1861 is coupled to the housing 102 of the inhaler device 100 such that it can move along the predetermined path 1863 relative to the housing 102. As best shown on FIG. 19B, the predetermined path 1863 is formed by a recess of an inner surface of the housing 102 and is linear. In another embodiment (not shown), the predetermined path 1863 may be non-linear. For example, the predetermined path 1863 may be curved to allow for better space efficiency within the inhaler device 100 and/or adjustment of the mechanical properties of the tensioning mechanism 1851 over the lifetime of the device.

The compression spring 1858 is disposed to act between the slider 1861 and the housing 102, providing a force that acts in the direction of the predetermined path 1863. A first end 1807 of the compression spring 1858 is attached or secured to the slider 1861 and a second end 1809 of the compression spring 1858 is attached or secured to the housing 102. The top sheet 166A of the blister strip 160A passes from the first index spool 131A, around the slider 1861, and is attached to the take-up hub 1852 as described above. The axial force of the compression spring 1858 pushes the slider 1861 in a direction away from the first index spool 131A and the take-up hub 1852. Stated another way, the compression spring 1858 is biased to push the slider 1861 away from each of the first index spool 131A and the take-up hub 1852. As a result, the length of the top sheet 166A between the first index spool 131A and the take-up hub 1852 is affected by the position of the slider 1861 within its available travel along the predetermined path 1863. With reference to FIG. 19A, when the compression spring 1858 is somewhat relaxed and at a longer length the slider 1861 is spaced further away from each of the first index spool 131A and the take-up hub 1852, whereby there is a greater or longer length of the top sheet 166A that extends between the first index spool 131A and the take-up hub 1852. Conversely, with reference to FIG. 19B, when the compression spring 1858 is compressed and at a shorter length the slider 1861 is spaced closer to the first index spool 131A and the take-up hub 1852, whereby there is a shorter length of the top sheet 166A that extends between the first index spool 131A and the take-up hub 1852.

During each dose, the first index spool 131A rotates in a first direction (for instance, clockwise) with its index gear, such as the first index gear 130A, and the take-up hub 1852 rotates in a second opposing direction (for instance, counter-clockwise) with its top sheet take-up gear, such as the top sheet take-up gear 150A as described above. As the first index spool 131A and the take-up hub 1852 are driven in opposing directions, the top sheet 166A is pulled around the slider 1861, peeling it away from the bottom sheet 162A which is moving clockwise with the first index spool 131A. Due to the wrapping effect described above, as the take-up hub 1852 rotates, the radial position of the top sheet 166A of the blister strip 160A increases. As a result of this increased radial position, the take-up hub 1852 attempts to peel a longer length of the top sheet 166A and the tension on the top sheet 166A increases due to a change in the peeling angle between the top sheet 166A of the blister strip 160A and the bottom sheet 162A of the first blister strip 160A. When the take-up hub 1852 starts to pull the top sheet 166A further, the reaction force at the peeling edge increases and thus the tension in the top sheet 166A increases. The tensioning mechanism 1851 ensures that the top sheet 166A of the blister strip 160A is peeled the same amount each dose. In order to do so, the increased tension in the top sheet 166A acts to pull or move the slider 1861 towards the index spool 131, against the force from the compression spring 1858. As the slider 1861 moves towards the first index spool 131A, the length of the top sheet 166A extending between the first index spool 131A and the take-up hub 1852 will decrease and thereby reduce the reaction force at the peeling edge. The movement of the slider 1861 is illustrated via a comparison of FIG. 19A and FIG. 19B. Axial movement of the slider 1861 (from the increased tension in the top sheet 166A) axially compresses the compression spring 1858 to reduce the tension along the top sheet 166A, and axial compression of the compression spring 1858 moves the slider 1861 closer to each of the first index spool 131A and the take-up hub 1852. The tensioning mechanism stabilizes when the force from the compression spring 1858 balances the top sheet tension, and the top sheet tension balances the reaction force at the peeling edge. In this way, the tension in the top sheet 166A is maintained at a relatively consistent level by the tensioning mechanism throughout the lifetime of the device. The tensioning mechanism 1851 stabilizes or balances the tension in the top sheet 166A via compression of the compression spring 1858 and movement of the slider 1861.

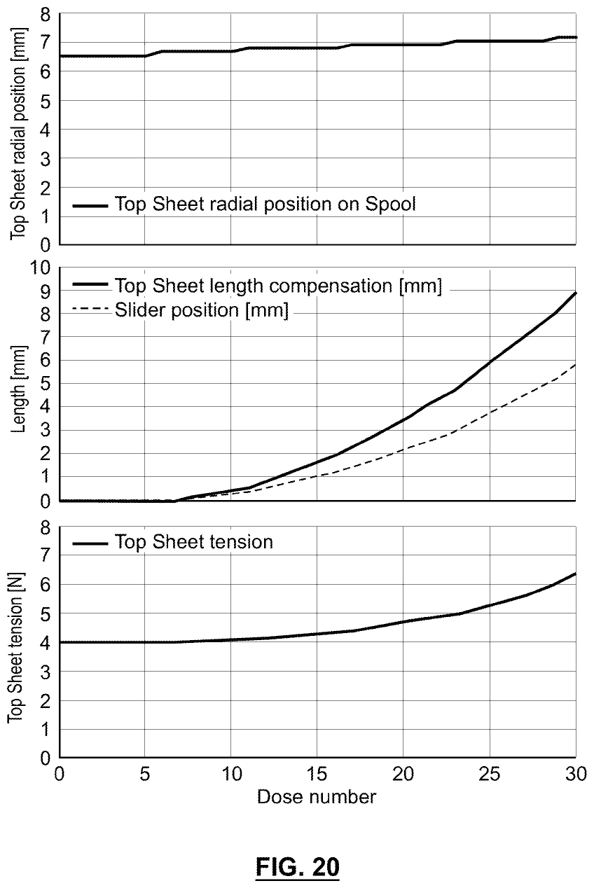

FIG. 20 illustrates performance characteristics of the tensioning mechanism 1851. The graph of FIG. 20 shows the increase in radial position of the top sheet 166A over the device lifetime, and the cumulative length compensation of the top sheet 166A that is achieved by the movement of the slider 1861. The graph also shows the estimated tension in the top sheet 166A over the device lifetime. There may be a gradual increase in top sheet tension due to the stiffness of the compression spring 1858, but as shown the top sheet tension is substantially consistent or constant over the device lifetime.

It will be understood by one of ordinary skill in the art that the relative dimensions and/or arrangement of the various components of the tensioning mechanism 1851 may vary from that shown in the embodiment of FIGS. 18-20. Different performance characteristics of the tensioning mechanism 1851 may be achieved with differences in component geometry, positioning and compression spring specification. For example, a compression spring with a longer solid height and a lower stiffness may be utilized. Such a compression spring may take up more space within the inhaler device but may result in more consistent top sheet tension over the device life. Variation in the relative dimensions and/or arrangement of the various components provides alternative options which may be desired in some applications, depending on factors such as space requirements, manufacturing or assembly methods, and the like.

When the compression spring 1858 is in its initially assembled state within the inhaler device 100 and the top sheet 166A is attached to take-up hub 1852, the compression spring 1858 is deflected or slightly compressed from its uncompressed length so that the compression spring 1858 applies a preload force. The preload force ensures that tension in the top sheet 166A of the blister strip 160A is sufficiently high to peel it away from the bottom sheet 162A of the blister strip 160A at the start of the device life. The specifications of the compression spring 1858 and the surrounding geometry should be configured to provide a minimum tension in the top sheet 166A of the blister strip 160A that is higher than the maximum force required to peel the top sheet 166A of the first blister strip 160A from the bottom sheet 162A of the blister strip 160A.

In the embodiment depicted in FIGS. 18-20, the compression spring 1858 is a single spring. However, the compression spring 1858 may include more than one spring. For example, in another embodiment, the compression spring 1858 includes exactly two springs used in parallel or coaxially. Utilization of two compression springs in parallel or coaxially may allow the solid height of the springs to be shorter for the same combined stiffness, and therefore the physical footprint of tensioning mechanism can be reduced.

Further, in another embodiment, one or more extension springs may be utilized as an alternative to the compression spring 1858. The extension spring would be positioned relative to the slider such that axial movement of the slider axially extends the extension spring towards the first index spool 131A to reduce the tension along the top sheet of the blister strip. The extension spring may be desired for some device layouts in terms of space efficiency. Further, in another embodiment, one or more torsion springs may be utilized as an alternative to the compression spring 1858. If an extension or torsion spring was utilized as an alternative to the compression spring 1858, the spring(s) would be acting between the slider and the housing, and providing a force in the direction that moves the slider away from the index gear (i.e., in a direction that would increase the length of the top sheet between the index gear and the take-up hub). In the case of a torsion spring, it may be desirable to have the slider moving in an arc-shaped path rather than a linear path, so that the slider stays concentric to the axis of the torsion spring and maintain more consistent operation.

While the take-up hub 1852 is rotationally driven in an opposing direction from the index gear 130A in the embodiment of FIGS. 18-20, in another embodiment the take-up hub 1852 may be rotationally driven in the same direction as the index gear 130A via the addition of an idler gear (not shown) disposed between the index gear 130A and the take-up gear 150A. When the index gear 130A and the take-up hub 1852 both rotate in the same direction (i.e., the first or clockwise direction), the top sheet 166A wraps around the take-up hub 1852 in the first direction which brings the angle of the top sheet 166A closer to the angle of travel of the slider 1861. As a result, the same amount of slider travel would result in a greater change in length of the top sheet 166A. The reduced overall travel of the slider 1861 results in a smaller physical footprint of the tensioning mechanism and/or more consistent sheet tension.

Contrary to the tensioning mechanism 151A, the components of the tensioning mechanism 1851 are not held or contained within the take-up hub 1852. The uncontained or exposed nature of the slider 1861 and the compression spring 1858 allows a range of spring sizes and specifications to be incorporated without changing geometry or size of the take-up hub 1852, which may be useful to optimize the tensioning mechanism for different blister strip designs and specifications. The uncontained or exposed nature of the slider 1861 and the compression spring 1858 also allows improved inspection of the success of the assembly process and the status of the tensioning mechanism during assembly, reducing the risk of assembly errors being undetected.

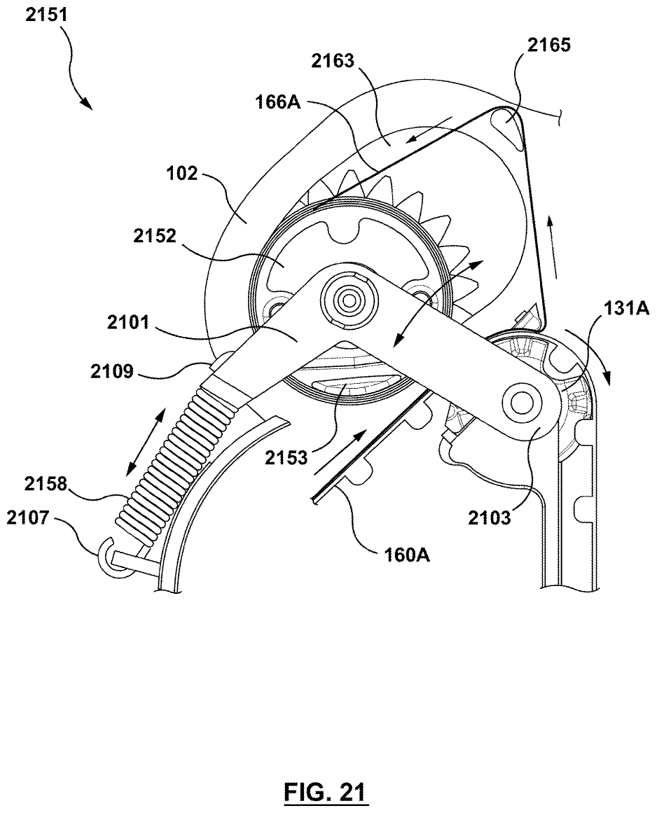

FIGS. 21-23B illustrate another embodiment of a tensioning mechanism or subassembly 2151 that may be utilized within the inhaler device 100. Similar to the tensioning mechanisms described above, the tensioning mechanism 2151 functions to peel a top sheet 166A from a blister strip 160A, in a manner that maintains consistent peeling distance or amount over the device lifetime. The tensioning mechanism 2151 also functions to maintain sheet tension of the top sheet 166A over the device lifetime. The tensioning mechanism 2151 is associated with a top sheet take-up gear, such as the first top sheet take-up gear 150A, for winding up the top sheet 166A of the blister strip 160A. It should be understood that in the inhaler device 100 each of the first and second tensioning mechanisms 151A, 151B may be replaced by a tensioning mechanism 2151 without departing from the scope of the present disclosure.

In this embodiment, a top sheet take-up gear is integrally formed with or secured to a take-up hub 2152 to rotate as an assembly. The top sheet 166A of the blister strip 160A is secured or attached to the take-up hub 2152 via a hook 2153 such that as the take-up hub 2152 rotates, the top sheet 166A of the blister strip 160A wraps around it. As described above, as the take-up hub 2152 completes multiple rotations over the lifetime of the inhaler device 100, the top sheet 166A of the blister strip 160A wraps over itself multiple times, causing its radial position on the take-up hub 2152 to increase.

The tensioning mechanism 2151 includes a pin 2165 fixed to and extending from an inner surface, wall or component of the housing 102, a bracket 2101, and at least one extension spring 2158 attached to the bracket 2101. The tensioning mechanism 2151 is configured to adjust the length of the top sheet 166A between the take-up hub 2152 and the first index spool 131A to maintain consistent tension on the top sheet 166A and thereby to compensate for the wrapping effect and component tolerances. As will be described in more detail herein, the tensioning mechanism 2151 maintains consistent tension on the top sheet 166A by permitting movement of the take-up hub 2152 (and the top sheet 166A mounted thereon) via the extension spring 2158, wherein the extension spring 2158 is biased in a direction generally away from the take-up hub 2152 and the top sheet 166A wrapped therearound. The take-up hub 2152 is coupled to the housing 102 of the inhaler device 100 so as to permit movement of the take-up hub 2152 relative to the housing 102 along a predetermined path 2163. The pin 2165 is configured to receive and guide an intermediate portion of the top sheet 166A, the intermediate portion being disposed between the first index spool 131A and the take-up hub 2152. Increased tension along the top sheet 166A results in movement of the take-up hub 2152 along the predetermined path 2163, and the movement of the take-up hub 2152 axially extends or stretches the extension spring 2158 to reduce the tension along the top sheet 166A.

More particularly, the take-up hub 2152 is coupled to the housing 102 of the inhaler device 100 such that it can move along the predetermined path 2163 relative to the housing 102. The predetermined path 2163 is formed by a curved slot of an inner surface of the housing 102. The take-up hub 2152 extends through the curved slot defining the predetermined path 2163. The curved slot forming the predetermined path 2163 is concentric with an axis of rotation of the first index spool 131A, so that the take-up hub 2152 travels in an arc that is concentric with the axis of rotation of the first index spool 131A. As a result, the take-up gear 150A will remain meshed with the index gear 130A as the take-up hub 2152 moves along the predetermined path 2163.

The pin 2165 radially extends from the inner surface of the housing 102 and is fixed to the housing. The top sheet 166A passes from the first index spool 131A, around the pin 2165, and is then attached to the take-up hub 2152. The pin 2165 is disposed adjacent to an end of the curved slot forming the predetermined path 2163. As will be described in more detail below, the pin 2165 is positioned within the housing 102 such that movement of the take-up hub 2152 results in a change in length of the top sheet 166A between the take-up hub 2152 and the first index spool 131a.

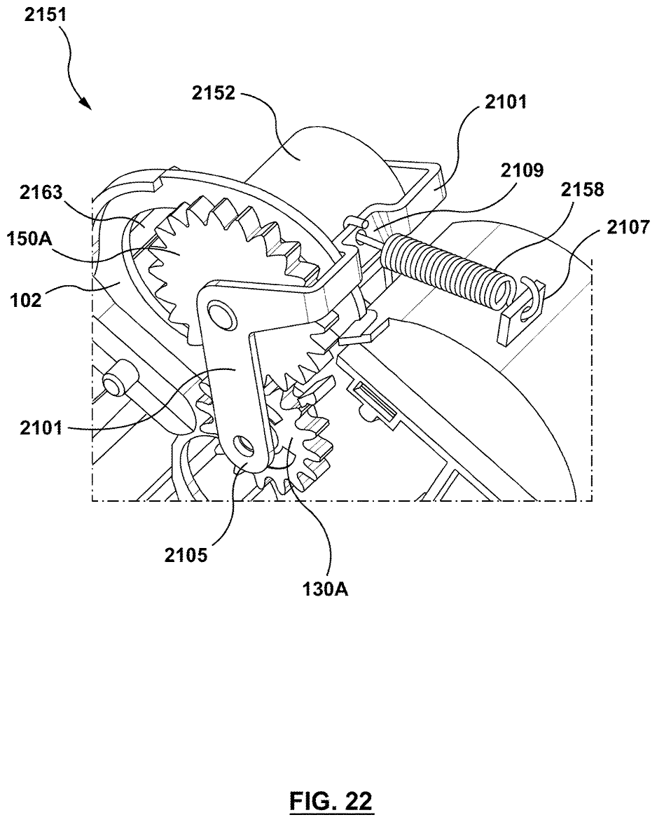

The take-up hub 2152 is mechanically linked or coupled to the housing 102 via the bracket 2101. The take-up hub 2152 is coupled to the bracket 2101 such that the take-up hub 2152 rotates relative to the bracket 2101 when the take-up hub 2152 is rotationally driven to wind up the top sheet 166A. Similarly, the index gear 130A is coupled to the bracket 2101 such that the index gear 130A rotates relative to the bracket 2101 when the index gear 130A is rotationally driven to advance the blister strip 160A. The bracket 2101 has a first end 2103 (shown in FIG. 21) and a second end 2105 (shown in FIG. 22) that are coupled to the first index spool 131A in such a manner as to permit rotation of the bracket 2101 relative to the first index spool 131A. Therefore, as the bracket 2101 rotates relative to the housing 102, the center of the take-up hub 2152 moves in the predetermined path 2163 of the curved slot of the housing 102. The bracket 2101 is permitted to rotate relative to the housing 102, and the bracket 2101 rotates or pivots about the index gear 130A when the take-up hub 2152 travels within the predetermined path 2163.

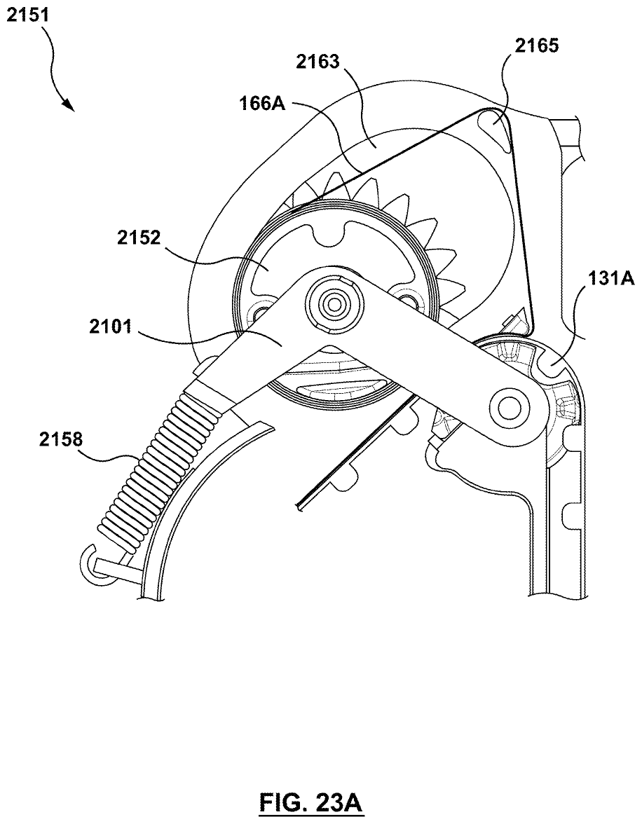

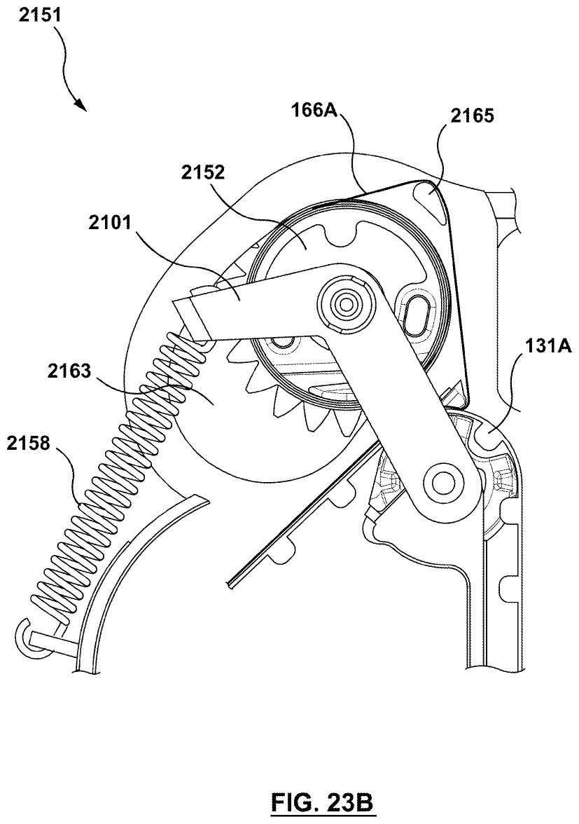

The extension spring 2158 is disposed to act between the bracket 2101 and the housing 102, providing a force that pulls the take-up hub 2152 toward one end of the predetermined path 2163 of the curved slot of the housing 102, away from the pin 2165. As best shown on FIG. 22, a first end 2107 of the extension spring 2158 is attached or secured to the housing 102 and a second end 2109 of the extension spring 2158 is attached or secured to the bracket 2101. The top sheet 166A of the blister strip 160A passes from the first index spool 131A, around the pin 2165, and is attached to the take-up hub 2152 as described above. The axial force of the extension spring 2158 pulls the take-up hub 2152 in a direction away from the pin 2165. Stated another way, the extension spring 2158 is biased to position the take-up hub 2152 at an end of the curved slot which is opposite from the pin 2165. As a result, the length of the top sheet 166A between the first index spool 131A and the take-up hub 2152 is affected by the position of the take-up hub 2152 within its available travel along the predetermined path 2163. With reference to FIG. 23A, when the extension spring 2158 is compressed and at a shorter length the take-up hub 2152 is spaced further away from the pin 2165, whereby there is a greater or longer length of the top sheet 166A between the first index spool 131A and the take-up hub 2152. Conversely, with reference to FIG. 23B, when the extension spring 2158 is stretched and at a longer length and the take-up hub 2152 is spaced closer to the pin 2165, whereby there is a shorter length of the top sheet 166A between the first index spool 131A and the take-up hub 2152.

During each dose, the first index spool 131A rotates in a first direction (i.e., clockwise) with the index gear 130A, and the take-up hub 2152 rotates in a second opposing direction (i.e., counterclockwise) with the top sheet take-up gear 150A as described above. As the first index spool 131A and the take-up hub 2152 are driven in opposing directions, the top sheet 166A is pulled around the pin 2165, peeling it away from the bottom sheet 162A which is moving clockwise with the first index spool 131A. Due to the wrapping effect described above, as the take-up hub 2152 rotates, the radial position of the top sheet 166A of the blister strip 160A increases. As a result of this increased radial position, the take-up hub 2152 attempts to peel a longer length of the top sheet 166A and the tension on the top sheet 166A increases due to a change in the peeling angle between the top sheet 166A of the blister strip 160A and the bottom sheet 162A of the blister strip 160A. As the take-up hub 2152 starts to pull the top sheet 166A further, the reaction force at the peeling edge increases and thus the tension in the top sheet 166A increases. The tensioning mechanism 2151 ensures that the top sheet 166A of the blister strip 160A is peeled the same amount each dose. The increased tension in the top sheet 166A acts to pull or move the take-up hub 2152 along the predetermined path 2163 defined by the curved slot of the housing 102 towards the pin 2165, against the force from the extension spring 2158. The bracket 2101 rotates relative to the housing 102 as the take-up hub 2152 moves along the predetermined path 2163. As the take-up hub 2152 moves towards the first index spool 131A and the pin 2165, the length of the top sheet 166A between the first index spool 131A and the take-up hub 2152 will decrease and thereby reduce the reaction force at the peeling edge. The movement of the take-up hub 2152, rotation of the bracket 2101, and deformation of the extension spring 2158 are illustrated via a comparison of FIG. 23A and FIG. 23B. Movement of the take-up hub 2152 (from the increased tension in the top sheet 166A) axially extends or stretches the extension spring 2158 to reduce the tension along the top sheet 166A, and the take-up hub 2152 moves closer to each of the first index spool 131A and the pin 2165. The tensioning mechanism stabilizes when the force from the extension spring 2158 balances the top sheet tension, and the top sheet tension balances the reaction force at the peeling edge. In this way, the tension in the top sheet 166A is maintained at a relatively consistent level by the tensioning mechanism throughout the lifetime of the device. The tensioning mechanism 2151 stabilizes or balances the tension in the top sheet 166A via extension of the extension spring 2158 and movement of the take-up hub 2152.

When the extension spring 2158 is initially assembled state within the inhaler device 100 and the top sheet 166A is attached to take-up hub 2152, the extension spring 2158 is deflected or slightly extended from its compressed length so that the extension spring 2158 applies a preload force. The preload force ensures that the tension in the top sheet 166A of the blister strip 160A is sufficiently high to peel it away from the bottom sheet 162A of the blister strip 160A at the start of the device life. The specifications of the extension spring 2158 and the surrounding geometry should be configured to provide a minimum tension in the top sheet 166A of the blister strip 160A that is higher than the maximum force required to peel the top sheet 166A of the blister strip 160A from the bottom sheet 162A of the blister strip 160A.

As can best be derived in the perspective view of FIG. 22, the extension spring 2158 is aligned or disposed in line with a centerline of the take-up hub 2152 and thus a centerline of the top sheet 166A that wraps therearound. This alignment between the extension spring 2158 and the centerline of the top sheet 166A minimizes torque on the bracket 2101 as the bracket 2101 rotates with movement of the take-up hub 2152.

Contrary to the tensioning mechanism 151A, the components of the tensioning mechanism 2151 are not held or contained within the take-up hub 2152. The uncontained or exposed nature of the bracket 2101 and the extension spring 2158 allows a range of spring sizes and specifications to be incorporated without changing geometry or size of the take-up hub 2152, which may be useful to optimize the tensioning mechanism for different blister strip designs and specifications. The uncontained or exposed nature of the bracket 2101 and the extension spring 2158 also allows improved inspection of the success of the assembly process and the status of the tensioning mechanism during assembly, reducing the risk of assembly errors being undetected.

It will be understood by one of ordinary skill in the art that the relative dimensions and/or arrangement of the various components of the tensioning mechanism 2151 may vary from that shown in the embodiment of FIGS. 21-23B. Variation in the relative dimensions and/or arrangement of the various components provides alternative options which may be desired in some applications, depending on factors such as space requirements, manufacturing or assembly methods, and the like.

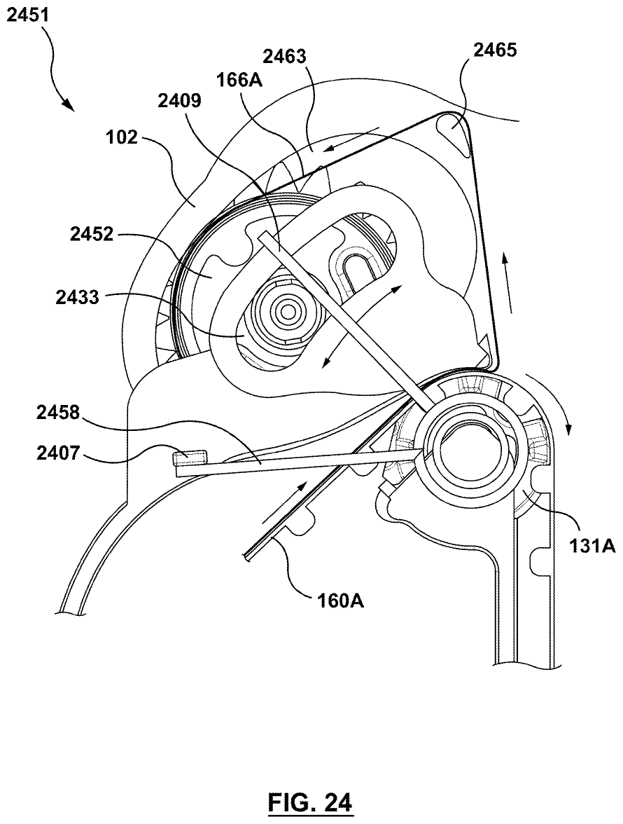

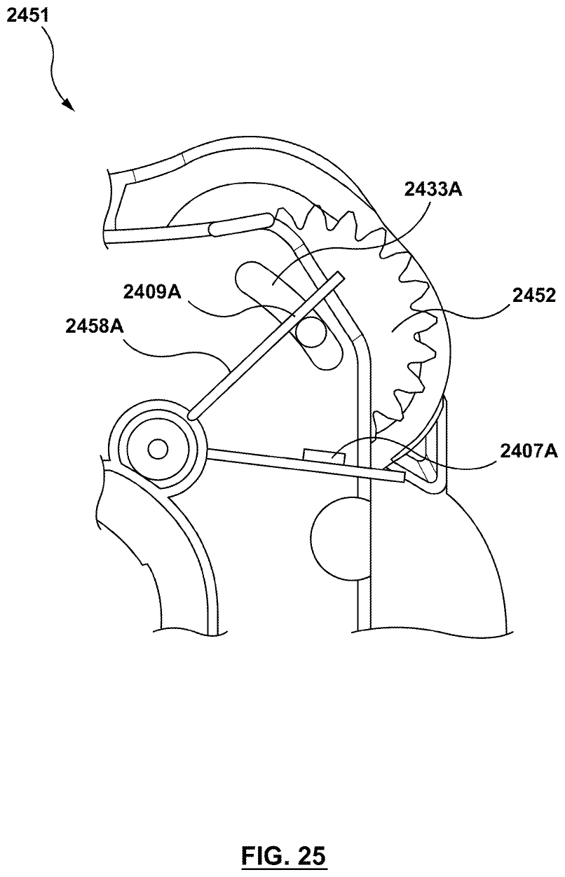

FIGS. 24 and 25 illustrate another embodiment of a tensioning mechanism or subassembly 2451 that may be utilized within the inhaler device 100. The tensioning mechanism 2451 functions similar to the tensioning mechanism 2151 but utilizes two torsion springs 2458, 2458A as an alternative to the extension spring 2158 and the bracket 2101. The first torsion spring 2458 is positioned to interact with a first side of a take-up hub 2452 and the second torsion spring 2458A is positioned to interact with a second opposing side of the take-up hub 2452. It is believed that utilization of two torsion springs acting on opposing sides of the take-up hub 2452 may provide better positional stability for the take-up hub 2452; however, in another embodiment hereof, the tensioning mechanism 2451 may include only a single torsion spring acting on a single side of the take-up hub 2452. The torsion springs 2458, 2458A function similar to the extension spring 2158 and provide a force to bias the take-up hub 2452 away from a pin 2465. Further, in this embodiment, the take-up hub 2452 is constrained within two arc-shaped slots 2433, 2433A in the housing 102 rather than via a bracket. Although the functional principles are similar to the tensioning mechanism 2151, the tensioning mechanism 2451 has a different physical footprint compared to the tensioning mechanism 2151, and different advantages in terms of assembly complexity, robustness, cost, and the like.

Tensioning mechanism 2451 includes several components that are the same as components of the tensioning mechanism 2151, and thus will not be described in detail. More particularly, the take-up hub 2452 is the same as the take-up hub 2152 and the pin 2465 is the same as the pin 2165, and a predetermined path 2463 is the same as the predetermined path 2163. The take-up hub 2452 is coupled to the housing 102 of the inhaler device 100 so as to permit movement of the take-up hub 2452 relative to the housing 102 along the predetermined path 2463. The pin 2465 is configured to receive and guide an intermediate portion of a top sheet 166A, the intermediate portion being disposed between the first index spool 131A and the take-up hub 2452. Increased tension along the top sheet 166A results in movement of the take-up hub 2452 along the predetermined path 2463.

A first leg 2407, 2407A of each torsion spring 2458, 2458A is fixed to the inner surface of the housing 102 and a second leg 2409, 2409A of each torsion spring 2458, 2458A is coupled to the take-up hub 2452 to move in conjunction with the take-up hub 2452 along predetermined path 2463 defined by the curved slot. A body of each torsion spring 2458, 2458A is disposed concentric to an axis of rotation of the index spool 131. Each torsion spring 2458, 2458A is biased to position the take-up hub 2452 at an end of the predetermined path 2463 defined by the curved slot which is opposite from the pin 2465. Movement of the take-up hub 2452 in a direction towards the pin 2465 twists the torsion springs 2458, 2458A to reduce the tension along the top sheet 166A of the blister strip 160A, as will be described in more detail below.

Each torsion spring 2458, 2458A is disposed to act between the take-up hub 2452 and the housing 102, providing a force that pulls the take-up hub 2452 toward the end of the predetermined path 2463 of the curved slot of the housing 102, that is away from the pin 2465. More particularly, a first leg 2407 of the first torsion spring 2458 is attached or secured to the housing 102 and a second leg 2409 of the first torsion spring 2458 is attached or secured to the take-up hub 2452, as shown in FIG. 24. Similarly, a first leg 2407A of the second torsion spring 2458A is attached or secured to the housing 102 and a second leg 2409A of the second torsion spring 2458A is attached or secured to the take-up hub 2452, as shown in FIG. 25. The top sheet 166A of the blister strip 160A passes from the first index spool 131A, around the pin 2465, and is attached to the take-up hub 2452. The force of the torsion springs 2458, 2458A pulls the take-up hub 2452 in a direction away from the pin 2465. Stated another way, the torsion springs 2458, 2458A are biased to position the take-up hub 2452 at an end of the curved slot which is opposite from the pin 2465. As a result, the length of the top sheet 166A between the first index spool 131A and the take-up hub 2452 is affected by the position of the take-up hub 2452 within its available travel along the predetermined path 2463.

During each dose, the first index spool 131A rotates in a first direction (i.e., clockwise) with the index gear 130A, and the take-up hub 2452 rotates in a second opposing direction (i.e., counter-clockwise) with the top sheet take-up gear 150A as described above. As the first index spool 131A and the take-up hub 2452 are driven in opposing directions, the top sheet 166A is pulled around the pin 2465, peeling it away from the bottom sheet 162A which is moving clockwise with the first index spool 131A. Due to the wrapping effect described above, as the take-up hub 2452 rotates, the radial position of the top sheet 166A of the blister strip 160A increases. As a result of this increased radial position, the take-up hub 2452 attempts to peel a longer length of the top sheet 166A and the tension on the top sheet 166A increases due to a change in the peeling angle between the top sheet 166A of the blister strip 160A and the bottom sheet 162A of the blister strip 160A. As the take-up hub 2452 starts to pull the top sheet 166A further, the reaction force at the peeling edge increases and thus the tension in the top sheet 166A increases. The tensioning mechanism 2451 ensures that the top sheet 166A of the blister strip 160A is peeled the same amount each dose. The increased tension in the top sheet 166A acts to pull or move the take-up hub 2452 along the predetermined path 2463 defined by the curved slot of the housing 102 towards the pin 2465, against the force from the torsion springs 2458, 2458A. As the take-up hub 2452 moves towards the first index spool 131A and the pin 2465, the length of the top sheet 166A between the first index spool 131A and the first take-up hub 2452 will decrease and thereby reduce the reaction force at the peeling edge. Movement of the take-up hub 2452 (from the increased tension in the top sheet 166A) twists the torsion springs 2458, 2458A to reduce the tension along the top sheet 166A, and the take-up hub 2452 moves closer to the each of the first index spool 131A and the pin 2465. The tensioning mechanism stabilizes when the force from the torsion springs 2458, 2458A balances the top sheet tension and the top sheet tension balances the reaction force at the peeling edge. In this way, the tension in the top sheet 166A is maintained at a relatively consistent level by the tensioning mechanism throughout the lifetime of the device. The tensioning mechanism 2451 stabilizes or balances the tension in the top sheet 166A via twisting of the torsion springs 2458, 2458A and movement of the take-up hub 2452.

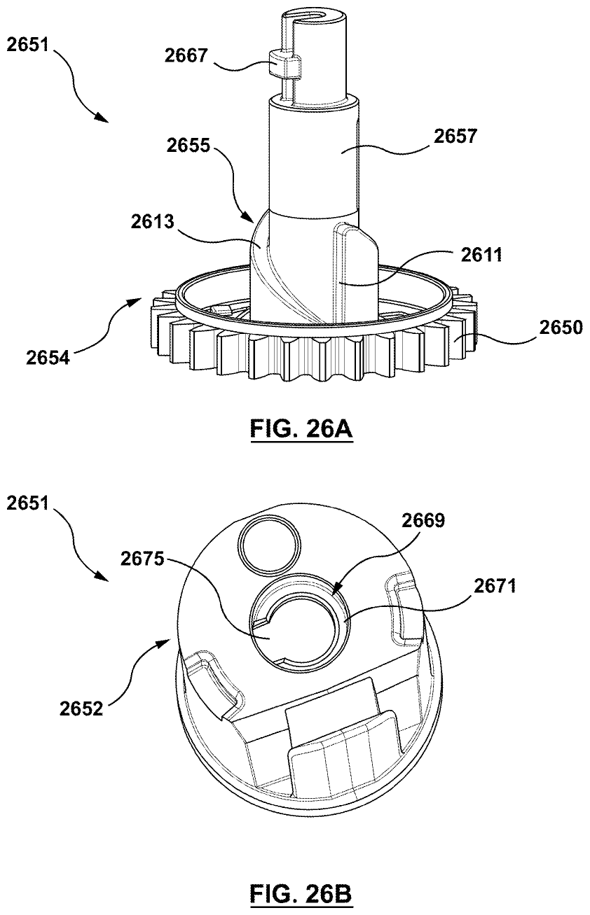

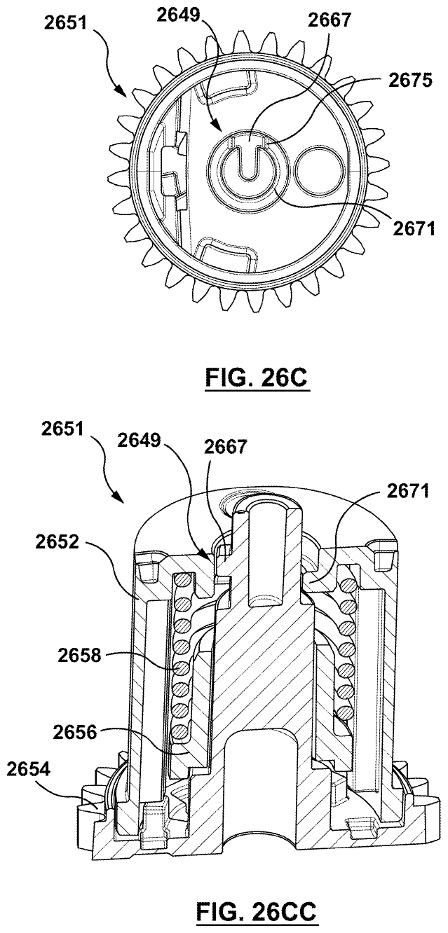

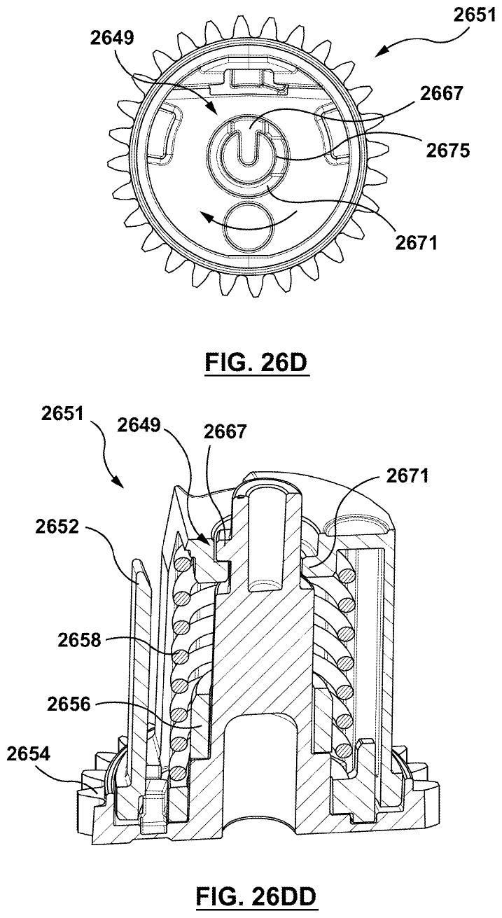

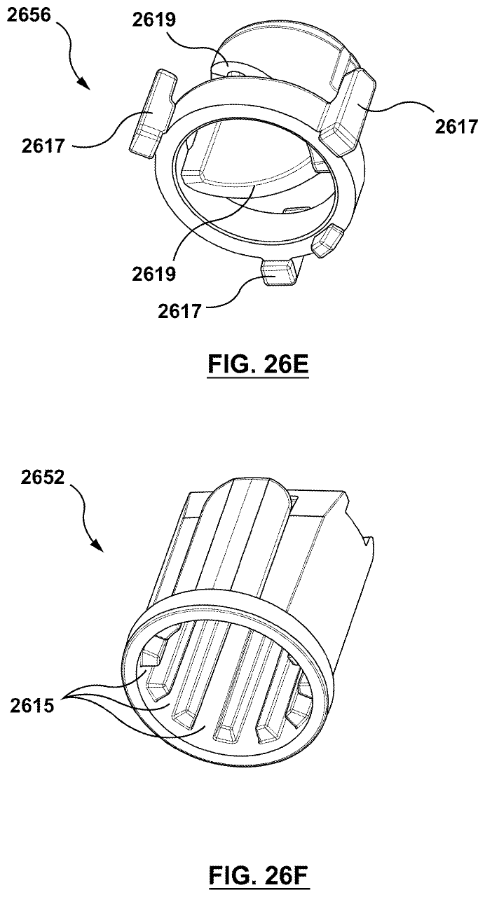

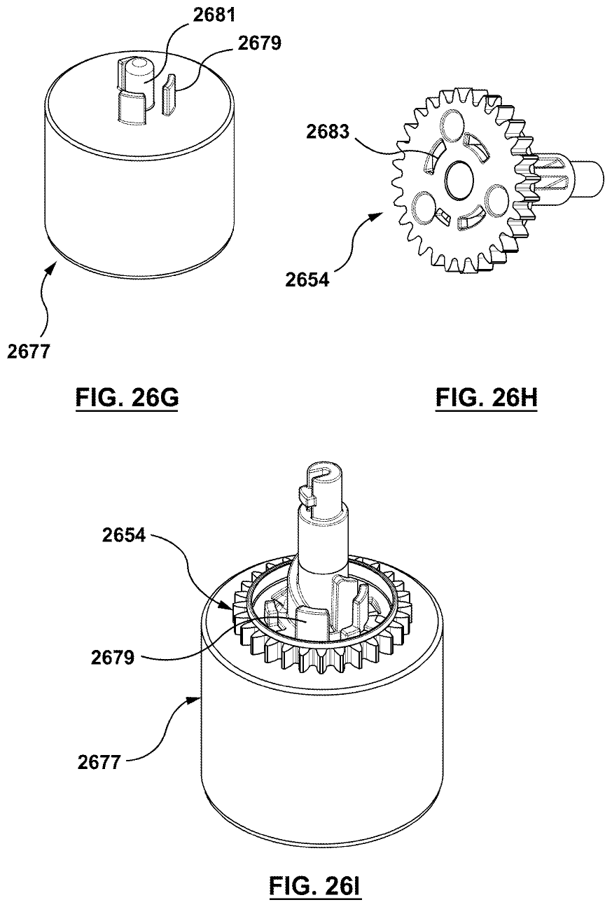

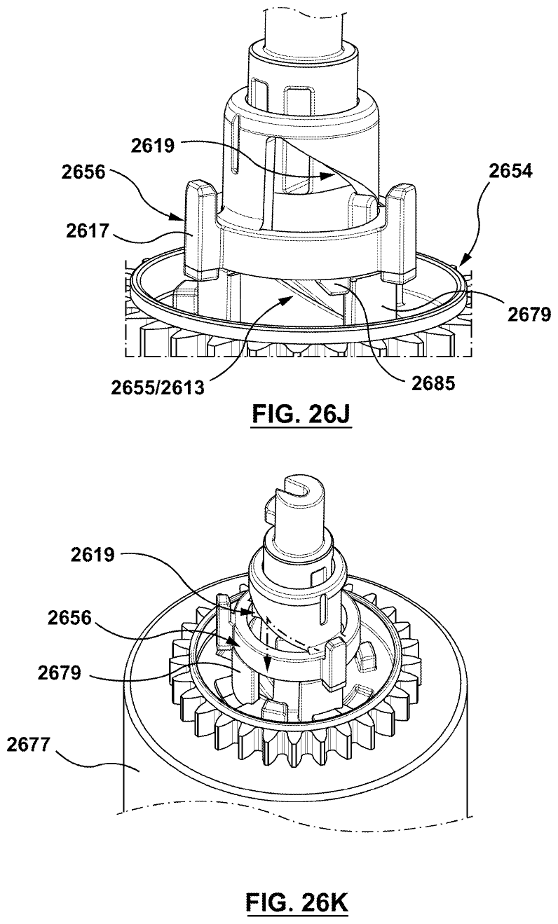

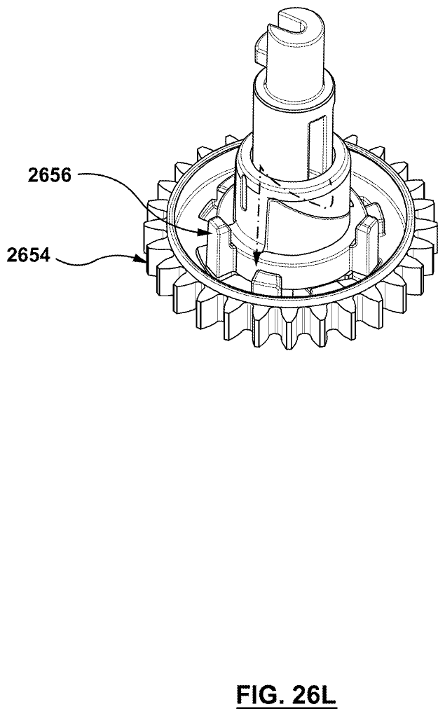

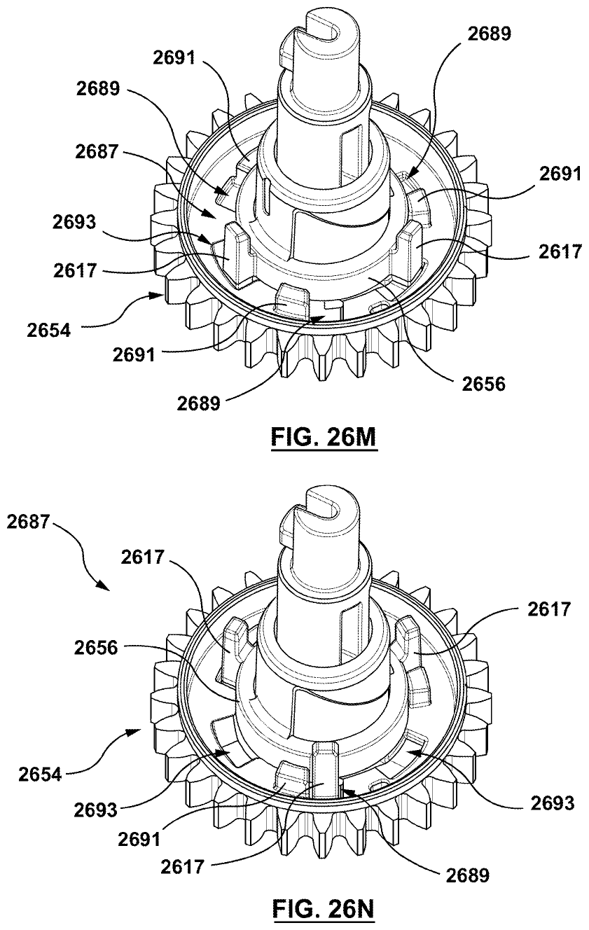

FIGS. 26A-26N illustrate another embodiment of a tensioning mechanism 2651 which operates the same as the tensioning mechanism 151A described above, but in this embodiment the retention feature that axially constrains the take-up hub relative to the base is a bayonet connection. Similar to the tensioning mechanism 151A, the tensioning mechanism 2651 includes a hub 2652, a base 2654 having a cam surface 2655 integrally formed thereon or fixed thereto, a nut 2656, a shaft 2657, and a compression spring 2658 that extends or is disposed between the nut 2656 and an end of the take-up hub 2652 that is spaced apart from the base 2654. The compression spring 2658 biases the nut 2656 downwards, towards the base 2654, into the cam surface 2655. The compression spring 2658 is disposed about or around the shaft 2657 and is longitudinally or axially adjacent to the nut 2656, and the take-up hub 2652 is disposed about or around the nut 2656 and the compression spring 2658. Stated another way, the take-up hub 2652 encircles or surrounds the nut 2656 and the compression spring 2658 which are contained therein.

The base 2654 is attached to or formed integrally with the first top sheet take-up gear 2650 to rotate as an assembly when the first top sheet take-up gear 2650 is rotationally driven. For example, the base 2654 may include a plurality of gear teeth integrally formed with or fixed to an outer circumferential surface thereof to form the first top sheet take-up gear 2650. The shaft 2657 extends from the base 2654 and is attached to or formed integrally with the base 2654 to rotate therewith. Thus, the shaft 2657, the base 2654, and the first top sheet take-up gear 2650 rotate as an assembly when the first top sheet take-up gear 2650 is rotationally driven. The cam surface 2655 of the shaft 2657 includes alternating sections of vertical surfaces 2611 and inclined surfaces 2613. Vertical surfaces 2611 extend generally parallel to a longitudinal axis of the shaft 2657. In this embodiment, the cam surface 2655 includes a total of two vertical surfaces 2611 disposed at opposing locations on the shaft 2657, and two inclined surfaces 2613 disposed at opposing locations on the shaft 2657. The inclined surfaces 2613 extend helically around the shaft 2657.

The take-up hub 2652 is axially constrained relative to the base 2654 via a bayonet connection 2649 between the shaft 2657 and the take-up hub 2652. Although the bayonet connection 2649 is described and shown implemented into the tensioning mechanism 2651, which operates the same as the tensioning mechanism 151A, the bayonet connection 2649 may similarly be implemented into any tensioning mechanism embodiment described herein.

With reference to FIGS. 26A and 26B, the bayonet connection 2649 includes a male bayonet feature or radial extension 2667 which is attached to or integrally formed with the shaft 2657 and is thus attached to the base 2654. The radial extension 2667 is a projection that extends radially outwards from an outer surface of the shaft 2657. The bayonet connection 2649 also includes a female bayonet feature 2669 formed in the take-up hub 2652. The female bayonet feature 2669 includes an internal flange 2671 formed on an inner surface of the take-up hub 2652, and the internal flange 2671 has an axial slot 2675 formed therethrough. In the depicted embodiment, a single male bayonet feature and a single female bayonet feature are used, although a greater number may be used. The internal flange 2671 is a planar projection that extends radially inwards from the inner surface of the take-up hub 2652 to form an internal ledge or shelf. In an embodiment, the internal flange 2671 extends around the entire inner diameter of the take-up hub 2652 except for the width of the axial slot 2675. The axial slot 2675 is configured to allow passage of the radial extension 2667 therethrough. During assembly of the tensioning mechanism 2651, the radial extension 2667 and the axial slot 2675 are circumferentially or rotationally aligned when the take-up hub 2652 is axially assembled or combined with the base 2654 so that the radial extension 2667 passes through the axial slot 2675. During assembly of the tensioning mechanism 2651, the bayonet connection 2649 is in an open or unlocked state.

More particularly, the open or unlocked state of the bayonet connection 2649 is shown in FIGS. 26C and 26CC. FIG. 26C illustrates a top view of the tensioning mechanism 2651 in the open or unlocked state, while FIG. 26CC is a cross-sectional view of FIG. 26C. The radial extension 2667 and the axial slot 2675 are circumferentially or rotationally aligned as best shown in FIG. 26C. The radial extension 2667 does not abut against or contact the internal flange 2671, because the radial extension 2667 is disposed through the axial slot 2675 of the flange 2671. In the open or unlocked state, the take-up hub 2652 is not axially constrained relative to the base 2654. Stated another way, with no force is applied thereto, the compression spring 2658 would act to push the take-up hub 2652 axially away from the base 2654 when the bayonet connection 2649 is in the open or unlocked state.

The take-up hub 2652 is configured to be rotated an amount relative to the base 2654 to lock the take-up hub 2652 and the base 2654 axially together, in order to resist the compression spring load of the compression spring 2658. The closed or locked state of the bayonet connection 2649 is shown in FIGS. 26D and 26DD, after the take-up hub 2652 is rotated an amount relative to the base 2654 to lock the take-up hub 2652 and the base 2654 axially together. FIG. 26D illustrates a top view of the tensioning mechanism 2651 in the closed or locked state, while FIG. 26DD is a cross-sectional view of FIG. 26D. The radial extension 2667 and the axial slot 2675 are no longer circumferentially or rotationally aligned as best shown in FIG. 26D. The radial extension 2667 abuts against a top surface of the internal flange 2671 as best shown in FIG. 26DD. Although the compression spring 2658 pushes against the take-up hub 2652, the take-up hub 2652 is axially constrained relative to the base 2654 in the closed or locked state because the internal flange 2671 functions as an axial stop and prevents axial movement of the take-up hub 2652. In the closed or locked state, the tensioning mechanism 2651 is a stable subassembly which can be installed into an inhaler device. In the embodiment of FIGS. 26A-26N, the take-up hub 2652 is configured to be rotated approximately ninety degrees (90°) relative to the base 2654 in order to transition the bayonet connection 2649 from the open or unlocked state to the closed or locked state. However, this is only exemplary and other relative rotation amounts may be utilized. For example, the rotation for locking may be less than ninety degrees (90°) provided that the bayonet features are adequately engaged, or may be more than ninety degrees (90°) provided that there is sufficient space for subsequent rotation of the take-up hub 2652 during operation.

When the take-up hub 2652 is rotated to transition the bayonet connection 2649 from the open or unlocked state to the closed or locked state, the nut 2656 also rotates because it is engaged with the take-up hub 2652 to rotate therewith. More particularly, the nut 2656 will be described in more detail with reference to FIG. 26E and the take-up hub 2652 will be described in more detail with reference to FIG. 26F. FIG. 26E is a perspective view of the nut 2656 removed from the tensioning mechanism 2651 for sake of illustration only. The nut 2656 is disposed between and coupled to each of the take-up hub 2652 and the base 2654. The nut 2656 is coupled to the take-up hub 2652 via a splined coupling (similar to splined coupling 159A) such that the take-up hub 2652 rotates with the nut 2656 and no relative rotation is permitted therebetween. Stated another way, due to the splined coupling, the take-up hub 2652 is rotationally locked to the nut 2656 such that the nut 2656 and the take-up hub 2652 rotate as an assembly. The nut 2656 operates similarly to the nut 156A described herein but has a different structure. In this embodiment, the splined coupling includes a plurality of outwardly-extending ribs 2617 which project or extend radially outwards from an outer circumferential surface of a lower collar portion of the nut 2656 and each outwardly-extending rib is received within an axial slot 2615 of the take-up hub 2652. The take-up hub 2652 includes a plurality of axial slots 2615 formed on an inner circumferential surface of the take-up hub 2652. Each outwardly-extending rib 2617 is permitted to slide or move in an axial direction along an axial slot 2615 such that the nut 2656 is permitted to slide or move in an axial direction relative to the take-up hub 2652, but the outwardly-extending rib 2617 does not permit the nut 2656 to rotate relative to the take-up hub 2652.

Rather than the inwardly-extending ribs 119A of the nut 156A, the nut 2656 includes upper or cam follower portion that is disposed axially above the collar portion of the nut 2656. This cam follower portion of the nut 2656 includes a lower surface 2619 that mates with and corresponds to the helical inclined surfaces 2613 of the cam surface 2655 such that the cam follower portion of the nut 2656 is disposed onto and engages the cam surface 2655 of the base 2654.

When the take-up hub 2652 is axially assembled to the base 2654 prior to locking the bayonet connection 2649, the take-up hub 2652 also engages the nut 2656 such that no relative rotation is permitted therebetween. As a result, when the bayonet feature 2649 is locked, the nut 2656 is also rotated relative to the base 2654 which thereby reduces the amount of rotation or travel available for the nut 2656 during normal operation. As such, FIGS. 26G, 26H, and 26I depict a nest fixture or tool 2677 that may be temporarily coupled to the base 2654 in order to initially install or assemble the nut 2656 to the base 2654 before the take-up hub 2652 is added such that the nut 2656 rotated backwards from a target initial position by an amount equal to the locking rotation. As a result, after assembly is complete and the bayonet feature 2649 is locked (thereby rotating the nut 2656), the nut 2656 rotates forward to return to the target initial position once the take-up hub 2652 has been rotationally locked. When in the target initial position, the nut 2656 is biased fully down against the base 2654, at the bottom of the cam surface 2655. The use of the nest fixture 2677 maximizes the degree or amount of rotation of the nut 2656 relative to the base 2654 during normal use.

The nest fixture 2677 is shown in FIG. 26G. The nest fixture 2677 is configured to hold or retain the nut 2656 at an elevated height relative to the base 2654 and therefore, if the nut 2656 is biased against the cam surface 2655 of the base 2654, a rotational position as required for assembly. The nest fixture 2677 includes a plurality of upstands 2679 which are sized and configured to pass through a plurality of apertures 2683 formed in the base 2654, as shown on FIG. 26H. The inner and outer diameter of the upstands 2679 are configured to permit rotation of the nut 2656 and the take-up hub 2652 during the locking rotation of the bayonet connection 2649 without interfering with the splined coupling 2659 between the nut 2656 and the take-up hub 2652. The nest fixture 2677 also includes a central locating pin 2681 for centering the base 2654 onto the nest fixture 2677 when the base 2654 is disposed on a top surface of the nest fixture 2677. FIG. 26I illustrates the base 2654 disposed on a top surface of the nest fixture 2677, with the upstands 2679 of the nest fixture 2677 extending through the apertures 2683 of the base 2654.

FIG. 26J illustrates the nut 2656 disposed onto or supported by the plurality of upstands 2679 at a position that corresponds to ninety degrees (90°) back or backwards from the target initial position of the nut 2656. The nut 2656 is rotationally secured by a rotational stop 2685 disposed between one of the upstands 2679 of the nest fixture 2677 and the nut 2656. The rotational stop 2685 ensures that the nut 2656 does not rotate away from the engagement with the cam surface 2655.

With reference to FIG. 26K, as the take-up hub 2652 (which is not shown in FIG. 26K such that the nut 2656 is visible) is rotated to lock the bayonet feature 2649, the nut 2656 rotates with the take-up hub 2652 as described above and as a result travels up the inclined surfaces 2613 of the cam surface 2655. The nut 2656 travels up the inclined surfaces 2613 and over the apexes thereof as shown by the directional arrow in FIG. 26K, with each apex being located at a junction between an inclined surface 2613 and a vertical surface 2611. As the nut 2656 travels over the apexes of the cam surface 2655, the nut 2656 will be biased back down against the upstands 2679 of the nest fixture 2677 by the compression spring 2658 to the height defined by the upstands. When the tensioning mechanism 2651 is removed from the nest fixture 2677, the nut 2656 will continue to move towards the base 2654 due to the spring force of the compression spring 2658 until it reaches the target initial position (i.e., biased fully down against the base 2654, at the bottom of the cam surface 2655).

Turning to FIG. 26L, another feature of the tensioning mechanism 2651 is illustrated. More particularly, as a secondary means to the dose counter to alert the user, the tensioning mechanism 2651 is configured to output an audible click to alert the user of the inhaler that the device is spent (i.e., all doses of the blister strip(s) have been delivered). As the inhaler is operated and doses are sequentially delivered, the nut 2656 travels up the inclined surfaces 2613 of the cam surface 2655. At the end of the device life, the nut 2656 travels up the inclined surfaces 2613 and is disposed at the apexes thereof, with each apex being located at a junction between an inclined surface 2613 and a vertical surface 2611. If the user continues to operate the inhaler (i.e., via rotation of the mouthpiece cover 108) after all doses have been dispensed, the nut 2656 will exceed the allotted or permissible amount of rotation and travel over the apexes of the cam surface 2655 as shown by the directional arrow in FIG. 26L. As the nut 2656 travels over the apexes of the cam surface 2655 and drops off the inclined surfaces 2613, the nut 2656 will be biased back down to the base 2654 by the compression spring 2658, resulting in or outputting an audible click.

Turning to FIGS. 26M and 26N, another feature of the tensioning mechanism 2651 is illustrated. More particularly, tensioning mechanism 2651 includes a lock-out mechanism 2687 which is configured to prevent disassembly of the bayonet connection 2649 if the user continues to attempt to operate the inhaler device after all doses have been dispensed. Stated another way, the lock-out mechanism 2687 limits how much the take-up hub 2652 can be wound or rotated in order to prevent the male and female bayonet features of the bayonet connection 2649 from realigning and the take-up hub 2652 disassembling axially due to the force of the compression spring 2658 within the take-up hub 2652.

As previously described, the nut 2656 includes a plurality of outwardly-extending ribs 2617 which project or extend radially outwards from an outer circumferential surface of the nut 2656 for engaging with the take-up hub 2652. In this embodiment, the outwardly-extending ribs 2617 also function as stop features of the lock-out mechanism 2687. More particularly, in this embodiment, the nut 2656 includes a total of three outwardly-extending ribs 2617 which are circumferentially spaced apart at equal intervals, i.e., equidistantly spaced apart at 120° around the outer circumferential surface of the nut 2656. After all doses have been dispensed and the nut 2656 travels over the apexes of the cam surface 2655, as described above with respect to FIG. 26L, the outwardly extending ribs 2617 are disposed upon the base 2654 at a different position from which they started. The base 2654 includes a plurality of pockets 2689 that are configured to receive the outwardly-extending ribs 2617 at this end of life state. When the outwardly-extending ribs 2617 are received with the pockets 2689, any further rotation of the nut 2656 (and therefore the take-up hub 2652 rotationally locked thereto) is prevented. Although pockets 2689 are sufficient to receive the outwardly-extending ribs 2617 and prevent further rotation of the nut 2656, the lock-out mechanism 2687 may include a plurality of raised stop blocks 2691 on the base 2654 adjacent to the pockets 2689 to increase vertical engagement with the outward-extending ribs 2617.

FIG. 26M illustrates the start or initial position of the outwardly-extending ribs 2617 at the beginning of device life, while FIG. 26N illustrates the end position of the outwardly-extending ribs 2617 at the end of device life after all doses have been dispensed and the nut 2656 travels over the apexes of the cam surface 2655. In the start position of FIG. 26M, the outwardly-extending ribs 2617 of the nut 2656 are initially located in helically-swept clearance grooves 2693 and are clear to rotate during operation of the inhaler device. The base 2654 includes a total of three clearance grooves 2693 for receiving the three outwardly-extending ribs 2617. Each clearance groove 2693 is disposed midway between a pair of adjacent pockets 2689. In the end position of FIG. 26N, the nut 2656 has rotated 180°during operation of the inhaler device, dropped over the cam surface 2655, and the outwardly-extending ribs 2617 are disposed within the pockets 2689 and blocked against further rotation by stop blocks 2691 of the lock-out mechanism 2687.

The lock-out mechanism 2687 advantageously includes three equispaced rotational stop features, at a relatively large diameter, while maintaining the same rotation of the nut 2656 relative to the base 2654 during normal operation. The nut 2656 travels past the cam surface 2655 before the lock-out mechanism 2687 becomes active, and the audible click described in relation to FIG. 26J signals engagement of the lock-out mechanism 2687.

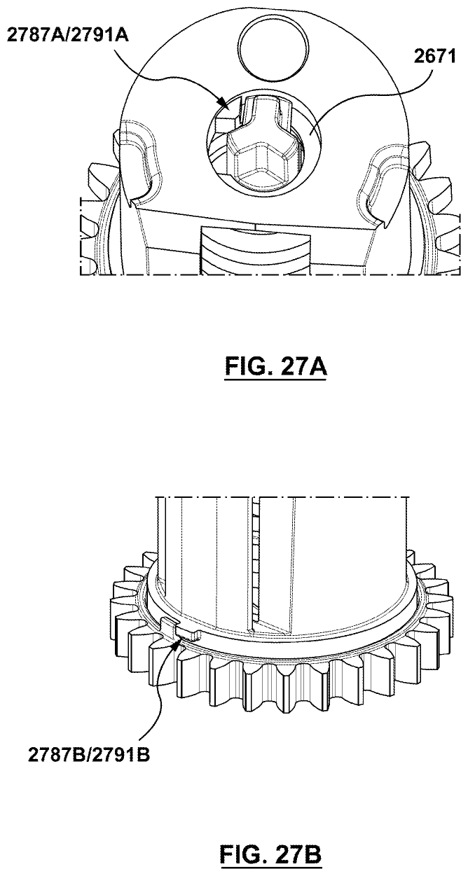

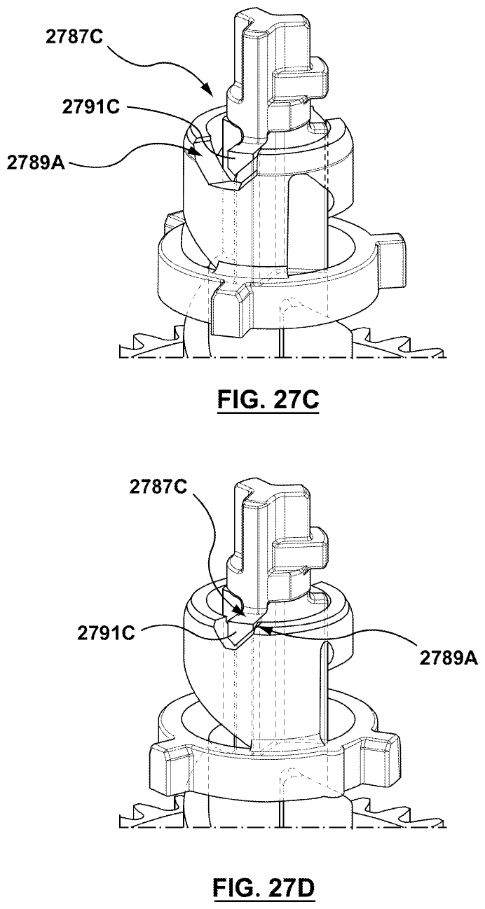

FIGS. 27A-27D illustrate alternative embodiments of a lock-out mechanism that may be integrated into the tensioning mechanism 2651 to prevent disengagement of the bayonet connection 2649. In FIG. 27A, a lock-out mechanism 2787A includes a rotational stop or block 2791A disposed at the bayonet connection 2649, at an end of the internal flange 2671. In FIG. 27B, a lock-out mechanism 2787B includes a rotational stop or block 2791B disposed at the lower perimeter of the hub 2652. In FIGS. 27C and 27D, a lock-out mechanism 2787C includes a rotational stop or block 2791C disposed on the shaft 2657 at a top end of the cam surface 2655 and pair of opposing cut-outs in a top end of the nut 2656. In this embodiment, the nut includes a first cut-out 2789A is relatively larger than a second cut-out 2789B. With reference to FIG. 27C, the first cut-out 2789A is configured to permit the nut 2656 to travel over or drop off the cam surface 2655 during assembly of the tensioning mechanism 2651 when the bayonet connection 2649 is locked. With reference to FIG. 27D, the second cut-out 2789B is configured to prevent the nut 2656 from traveling over or droping off the cam surface 2655 at the end of the device life, thereby preventing further rotation of the nut 2656 relative to the base 2654.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A tensioning mechanism for peeling a sheet from a blister strip for use in a dry powder inhaler device, the tensioning mechanism comprising:
a base;
a nut that includes at least one rib that engages with a cam surface;
a compression spring longitudinally axially adjacent to the nut; and
a take-up hub disposed about the nut and the compression spring, wherein the nut is disposed between and coupled to each of the take-up hub and the base, and wherein the take-up hub is rotationally constrained to the nut, and
wherein the take-up hub is configured to rotate relative to the base, and
wherein when the base is rotationally driven the nut is configured to interact with the compression spring and the cam surface to apply a torque onto the take-up hub, and
wherein when the take-up hub applies an opposing torque onto the nut, the nut moves along the cam surface and axially compresses the compression spring.

2. The tensioning mechanism of claim 1, wherein the take-up hub includes a hook on an outer surface thereof, the hook being configured to attach to an end of the sheet of the blister strip such that rotation of the take-up hub results in winding of the sheet of the blister strip around the take-up hub.

3. The tensioning mechanism of claim 1, wherein the take-up hub is axially constrained relative to the base.

4. The tensioning mechanism of claim 3, wherein the take-up hub is axially constrained relative to the base via a bayonet connection between the base and the take-up hub.

5. The tensioning mechanism of claim 4, wherein the bayonet connection includes a radial extension on the base and an internal flange in the take-up hub, the internal flange including an axial slot that is configured to allow passage of the radial extension therethrough.

6. The tensioning mechanism of claim 1, wherein the base includes a plurality of gear teeth integrally formed with or fixed to an outer circumferential surface of the base.

7. The tensioning mechanism of claim 1, wherein the cam surface includes alternating sections of vertical surfaces and inclined surfaces.

8. The tensioning mechanism of claim 7, wherein the at least one rib includes a plurality of ribs circumferentially spaced apart.

9. The tensioning mechanism of claim 1, wherein the cam surface is integrally formed with or fixed to a portion of the base, and wherein the at least one rib projects radially inwards from an inner circumferential surface of the nut, and wherein the take-up hub is rotationally locked to the nut.

10. The tensioning mechanism of claim 9, wherein the compression spring extends between the take-up hub and the nut.

11. The tensioning mechanism of claim 9, wherein the take-up hub is rotationally locked to the nut via an outwardly-extending rib which projects radially outwards from an outer circumferential surface of the nut and is received within an axial slot of the take-up hub.

12. The tensioning mechanism of claim 9, wherein the cam surface has a first outer diameter, and the compression spring has a second outer diameter, the first outer diameter being greater than the second outer diameter.

13. The tensioning mechanism of claim 1, wherein the cam surface is integrally formed with or fixed to a portion of the take-up hub, and wherein the at least one rib projects radially outwards from an outer circumferential surface of the nut, and wherein the base is rotationally locked to the nut.

14. The tensioning mechanism of claim 13, wherein a shaft extends from the base, the shaft being integrally formed with or fixed to the base, and wherein the base is rotationally locked to the nut via an inwardly-extending rib which projects radially inwards from an inner circumferential surface of the nut and is received within an axial slot of the shaft.

15. The tensioning mechanism of claim 1, wherein the compression spring extends between the nut and the base.

16. A dry powder inhaler device comprising:
a housing for receiving at least one blister strip for use in the dry powder inhaler device, the blister strip including a bottom sheet and a top sheet releasably secured to the bottom sheet;
the tensioning mechanism of claim 1 disposed within the housing; and,
an index spool which is rotationally driven in a first direction, wherein an outer surface of the index spool receives the bottom sheet of the blister strip,
wherein an outer surface of the take-up hub is attached to an end of the top sheet and rotation of the take-up hub in a second opposing direction causes the top sheet to wind around the outer surface of the take-up hub, and
wherein increased tension along the top sheet of the blister strip results in rotation of the take-up hub relative to the base in the first direction to reduce tension along the top sheet of the blister strip, and
wherein axial compression of the compression spring is transferred to torque applied to the take-up hub.

17. A dry powder inhaler device comprising:
a housing for receiving at least one blister strip for use in the dry powder inhaler device, the blister strip including a bottom sheet and a top sheet releasably secured to the bottom sheet;
an index spool which is rotationally driven in a first direction, wherein an outer surface of the index spool receives the bottom sheet of the blister strip;
a take-up hub which is rotationally driven in the first direction or a second opposing direction, wherein an outer surface of the take-up hub is attached to an end of the top sheet and rotation of the take-up hub causes the top sheet to wind around the outer surface of the take-up hub; and
a tensioning mechanism including a slider and at least one spring attached to the slider, wherein the tensioning mechanism is coupled to the housing so as to permit axial movement of the slider relative to the housing along a predetermined path,
wherein the slider is configured to receive an intermediate portion of the top sheet of the blister strip, the intermediate portion of the top sheet disposed between the index spool and the take-up hub, and
wherein increased tension along the top sheet of the blister strip results in axial movement of the slider along the predetermined path, and wherein axial movement of the slider axially compresses or extends the spring to reduce tension along the top sheet of the blister strip.

18. (canceled)

19. The dry powder inhaler device of claim 17, wherein the predetermined path is formed by a recess in an inner surface of the housing.

20. The dry powder inhaler device of claim 17, wherein the spring is a compression spring, and the compression spring is biased to push the slider away from each of the index spool and the take-up hub.

21. The dry powder inhaler device of claim 20, wherein axial compression of the compression spring moves the slider closer to the each of the index spool and the take-up hub.

22-40. (canceled)
